(19) United States
(12) Patent Application Publication
(10) Pub. No.: US 2023/0093708 A1
(43) Pub. Date: Mar. 23, 2023

(54) PROTEIN NANOPARTICLE DESIGN AND APPLICATION

(71) Applicant: Duke University, Durham, NC (US)

(72) Inventors: Michael Dzuricky, Durham, NC (US); Ashutosh Chilkoti, Durham, NC (US)

(21) Appl. No.: 17/908,415

(22) PCT Filed: Mar. 3, 2021
(86) PCT No.: PCT/US2021/020589

§ 371 Date: Aug. 31, 2022

Related U.S. Application Data
(60) Provisional application No. 62/985,174, filed on Mar. 4, 2020.

(51) Int. Cl. C07K 14/78; C07K 14/31; C07K 14/005

(52) U.S. Cl. CPC C07K 14/78; C07K 14/31; C07K 14/005; B82Y 5/00

(57) ABSTRACT

Described herein are protein nanoparticles comprising a fusion protein comprising at least one binding polypeptide and at least one unstructured polypeptide, in one aspect, the nanoparticles comprise a di-block of repeats of a core polypeptide, repeats of a corona polypeptide, and one or more binding proteins. The nanoparticles can be used as therapeutic agents, targeted-delivery agents, separation agents, or purification agents.

### CROSS REFERENCE TO RELATED APPLICATIONS

This application claims priority to U.S. Provisional Patent Application No. 62/985,174 filed on Mar. 4, 2020, which is incorporated by reference herein in its entirety.

### FEDERALLY SPONSORED RESEARCH

This invention was made with United States government support under National Science Foundation grant number DMR-17-29671. The United States government has certain rights in the invention.

### SEQUENCE LISTING

This application is filed with a Computer Readable Form of a Sequence Listing in accord with 37 C.F.R. § 1.821(c). The text file submitted by EFS, “028193-9339-WO01_sequence_listing_2-MAR-2021_ST25.txt,” was created on Mar. 2, 2021, contains 121 sequences, has a file size of 294 Kbytes, and is hereby incorporated by reference in its entirety.

### TECHNICAL FIELD

Described herein are protein nanoparticles comprising a fusion protein comprising at least one binding polypeptide and at least one unstructured polypeptide. In one aspect, the nanoparticles comprise a di-block of repeats of a core polypeptide, repeats of a corona polypeptide, and one or more binding proteins. The nanoparticles can be used as therapeutic agents, targeted-delivery agents, separation agents, or purification agents.

### BACKGROUND

The last few decades have seen an explosion of interest in the development of nanoparticle carriers for drug delivery, much of it for the treatment of solid tumors. Many different types of nanoparticles have been synthesized and evaluated in preclinical models for cancer therapy, including inorganic nanoparticles dendrimers, polymer nanoparticles and self-assembled nanostructures—micelles and polymersomes/liposomes—of polymers and lipids.

The general interest in this area is to achieve the fabled “magic bullet” drug—a drug that acts potently as intended without negative side effects. The problem with this ideal is that most drugs must balance potency against delivery obstacles posed by human physiology. Nanoparticles have attracted much attention from the “nanomedicine” community because they may be the answer to this challenge, for two reasons. First, they can be loaded with a range of small molecules with diverse physio-chemical properties, making them near universal carriers for small molecule drugs and imaging agents. Second, appropriately designed nanoparticles show good colloidal stability in blood and can circulate for extended periods of time. Therefore, the promise of nanocarriers is to understand how material properties on the nanoscale can alter the negative properties of developed drugs and endow them with desirable properties. Other desirable properties may include, resistance to drug clearance/breakdown, tissue specific targeting, increased cell internalization and increased solubility in serum.

Over the past 20 years, many different drug delivery systems have arisen, and some have progressed into approved therapies today. The most widely used systems are liposomal formulations, where the core of the liposome is loaded with drugs and therefore endows the encapsulated cargo with the properties of the liposome formulation. Liposomes are a great delivery vehicle because of their biocompatibility, ease of synthesis and high loading capacity.

However, for encapsulating more hydrophobic moieties, polymeric micelle systems are more advantageous as they have a larger volume/g that is hydrophobic and more control over morphology in comparison to liposomes. However, challenges with synthesis, controlling polydispersity of assembled populations, incorporating additional functionality, burst drug release in vivo and overall biocompatibility challenges have limited their clinical potential.

Two terms often used in the literature are “active” and “passive” targeting. Passive targeting is enhanced through optimization of the shape, size, and surface charge of nanoparticles to improve tumor accumulation due to the enhanced permeability and retention effect. Recently, particles with high aspect ratios and high flexibility, referred to as filomicelles, have attracted much research interest due to their long circulation time, high tumor penetration, and accumulation, and enhanced active target deliver. These particles are created via self-assembly or pattern-molding, which are convenient to create precise particle shapes, but are somewhat incompatible with protein drugs or the presentation of protein targeting ligands, as the conditions employed for their synthesis may denature proteins rendering them inactive in the body.

Passive targeting is a useful approach for locoregional targeting of solid tumors, but it does not directly target tumor cells, which are the ultimate destination of the drug or imaging agent. The rationale for creating targeted nanoparticles for cancer therapy or imaging stems from the fact that many tumors have surface proteins that are either overexpressed or—in a few instances—are uniquely expressed on the surface of tumor cells compared to normal, healthy cells. Homing the nanoparticle to tumor cells by decorating it with a ligand specific to a tumor-selective or tumor-specific marker—once the carrier has accumulated to a high enough concentration in the local environment of the tumor—can provide a second stage of tumor-cell specific targeting.

Active targeting utilizes a specific binding motif and targeting structure on the cell of interest to localize particles loaded with drug or imaging agent with a biophysical signal. A common approach to synthesize targeted nanocarriers is to functionalize the surface of the nanoparticle with a peptide or protein by covalent conjugation. This approach however provides limited control of ligand valency, and typically requires an excess of ligand to drive the reaction, and is hence expensive to scale up, and quality control and product validation remains a significant challenge.

What is needed are fusion proteins that can form nanoparticles for cellular targeting, drug delivery, and biomolecule purification.

### SUMMARY

One embodiment described herein is a composition comprising a protein nanoparticle comprising a fusion protein comprising at least one binding polypeptide and at least one unstructured polypeptide. In one aspect, the fusion protein comprises a plurality of unstructured polypeptides. In another aspect, the fusion protein comprises a plurality of targeting polypeptides. In another aspect, the unstructured polypeptides comprise a di-block peptide. In another aspect, the unstructured polypeptides comprise a di-block of a core polypeptide and a corona polypeptide. In another aspect, the unstructured polypeptides comprise COREₙ-CORONAₘ, where n is 20-200 repeats and m is 40-200 repeats. In another aspect, the core polypeptide comprises the sequence QYPSDGRG (SEQ ID NO:1); GRGDQPYQ (SEQ ID NO:2); GRGDSPYQ (SEQ ID NO:3); GRGDSPYS (SEQ ID NO:4); GRGDQPYS (SEQ ID NO:5); GRGDSP[3Y:V]S (SEQ ID NO:6); GRGDSP(Y:V]S (SEQ ID NO:7); or combinations thereof. In another aspect, the corona polypeptide comprises the sequence VPG[A:G]G (SEQ ID NO:8); VPGSG (SEQ ID NO:9); VPGVG (SEQ ID NO: 10); VPQQG (SEQ ID NO: 11); GRGDSPAS (SEQ ID NO:12); GRGDSPIS (SEQ ID NO:13); GRGDSPVS (SEQ ID NO:14); GRGDQPHN (SEQ ID NO:15); GRGDNPHQ (SEQ ID NO:16); GRGDSPV (SEQ ID NO:17); or combinations thereof. In another aspect, the core polypeptide comprises the sequence (RLP)ₙ (SEQ ID NO:1), where n is 20-200 repeats. In another aspect, the corona polypeptide comprises the sequence (ELP)m (SEQ ID NO:8), where m is 40-200 repeats. In another aspect, the di-block comprises: RLP40-ELP40 (SEQ ID NO:83); RLP40-ELP80 (SEQ ID NO:84); RLP40-ELP160 (SEQ ID NO:82); RLP60-ELP80 (SEQ ID NO:85); RLP80-ELP80 (SEQ ID NO:87); RLP80-ELP160 (SEQ ID NO:86); or RLP100-ELP80 (SEQ ID NO:88). In another aspect, the targeting polypeptide comprises 2 kDa to 100 kDa polypeptide. In another aspect, the targeting polypeptide comprises a type III domain from human fibronectin (Fn3) (SEQ ID NO:60); aFn3 domain from human tenascin C (Tn3) (SEQ ID NO:62); or a Z-domain of staphylococcal protein A (SEQ ID NO:64). In another aspect, the targeting polypeptide comprises a type III domain from human fibronectin (Fn3) (SEQ ID NO:60). In another aspect, the targeting polypeptide comprises a Fn3 domain from human tenascin C (Tn3) (SEQ ID NO:62). In another aspect, the targeting polypeptide comprises a Z-domain of staphylococcal protein A with a sequence comprising (SEQ ID NO:64). In another aspect, the core polypeptide is crosslinked.

Another embodiment described herein is a protein nanoparticle comprising a fusion protein comprising at least one binding polypeptide and at least one unstructured polypeptide. In one aspect, the fusion protein comprises a plurality of unstructured polypeptides. In another aspect, the fusion protein comprises a plurality of binding polypeptides. In another aspect, the unstructured polypeptides comprise a di-block peptide. In another aspect, the unstructured polypeptides comprise a di-block of a core polypeptide and a corona polypeptide. In another aspect, the unstructured polypeptides comprise COREₙ-CORONAₘ, where n is 20-200 repeats and m is 40-200 repeats. In another aspect, the core polypeptide comprises the sequence QYPSDGRG (SEQ ID NO:1); GRGDQPYQ (SEQ ID NO:2); GRGDSPYQ (SEQ ID NO:3); GRGDSPYS (SEQ ID NO:4); GRGDQPYS (SEQ ID NO:5); GRGDSP[3Y:V]S (SEQ ID NO:6); GRGDSP(Y:V]S (SEQ ID NO:7); or combinations thereof. In another aspect, the repeating core polypeptide sequence is interspersed by at least 1 but no more than 10 non-canonical amino acids selected from azidophenylalanine, acetylphenylalanine, propargyloxyphenylalanine, acetylphenylalanine, or azidohomoalanine. In another aspect, the corona polypeptide comprises the sequence VPG[A:G]G (SEQ ID NO:8); VPGSG (SEQ ID NO:9); VPGVG (SEQ ID NO: 10); VPQQG (SEQ ID NO: 11); GRGDSPAS (SEQ ID NO:12); GRGDSPIS (SEQ ID NO:13); GRGDSPVS (SEQ ID NO:14); GRGDQPHN (SEQ ID NO:15); GRGDNPHQ (SEQ ID NO:16); GRGDSPV (SEQ ID NO:17); or combinations thereof. In another aspect, the core polypeptide comprises the sequence (RLP)ₙ (SEQ ID NO:1), where n is 20-200 repeats. In another aspect, the corona polypeptide comprises the sequence (ELP)m (SEQ ID NO:8), where m is 40-200 repeats. In another aspect, the di-block comprises: RLP40-ELP40 (SEQ ID NO: 83); RLP40-ELP80 (SEQ ID NO: 84); RLP40-ELP160 (SEQ ID NO: 82); RLP60-ELP80 (SEQ ID NO: 85); RLP80-ELP80 (SEQ ID NO: 87); RLP80-ELP160 (SEQ ID NO: 86); or RLP100-ELP80 (SEQ ID NO: 88). In another aspect, the targeting polypeptide comprises 2 kDa to 100 kDa polypeptide. In another aspect, the binding polypeptide comprises a type III domain from human fibronectin (Fn3) (SEQ ID NO: 60); aFn3 domain from human tenascin C (Tn3) (SEQ ID NO: 62); or a Z-domain of staphylococcal protein A (SEQ ID NO: 64). In another aspect, the binding polypeptide comprises a comprises a type III domain from human fibronectin (Fn3) (SEQ ID NO: 60). In another aspect, the binding polypeptide c comprises a Fn3 domain from human tenascin C (Tn3) (SEQ ID NO: 62). In another aspect, the binding polypeptide comprises a Z-domain of staphylococcal protein A with a sequence comprising (SEQ ID NO:64). In another aspect, the core is covalently crosslinked using light or other click-chemistry compatible linkers. In another aspect, the core polypeptide is crosslinked. In another aspect, the nanoparticle encapsulates one or more small molecule drugs within its interior. In another aspect, the fusion protein further comprises a therapeutic protein. In another aspect, the composition is a therapeutic agent, targeted-delivery agent, separation agent, or purification agent. In another aspect, the binding polypeptide comprises an ErbB2 receptor binding protein (ANHP) (SEQ ID NO: 74). In another aspect, the binding polypeptide comprises a cell-binding peptide (GRGDSPAS) (SEQ ID NO: 76). In another aspect, the binding polypeptide comprises an adeno associated virus (AAV) binding protein (PKD2) (SEQ ID NO: 112). In another aspect, the binding polypeptide comprises an adenovirus (AdV) binding protein (CAR) (SEQ ID NO: 114). In another aspect, the binding polypeptide comprises a lentivirus (LV) binding protein (CR2) (SEQ ID NO: 116) or (CR3) (SEQ ID NO: 118). In another aspect, the binding polypeptide comprises an albumin binding protein (ABP) (SEQ ID NO: 120).

Another embodiment described herein is a therapeutic agent comprising the protein nanoparticle described herein.

Another embodiment described herein is a method of targeting a therapeutic to a cell comprising administering the protein nanoparticle described herein.

Another embodiment described herein is a method of delivering a therapeutic to a cell comprising administering the protein nanoparticle described herein.

Another embodiment described herein is a means for targeting a therapeutic to a cell comprising administering the protein nanoparticle described herein.

Another embodiment described herein is a means for delivering a therapeutic to a cell comprising administering the protein nanoparticle described herein.

Another embodiment described herein is a method for identifying a biomolecule comprising administering a protein nanoparticle described herein that binds to the biomolecule.

Another embodiment described herein is a method of purifying a biomolecule comprising using the protein nanoparticle described herein that binds to the biomolecule to isolate the biomolecule from a medium. In another aspect, further comprising a triggered phase separation of the binding polypeptide to isolate the biomolecule from contaminants, wherein the trigger is selected from a modulation of temperature, salinity, light, pH, pressure, concentration of the binding polypeptide, concentration of the biomolecule, application of electromagnetic or acoustic waves, or addition of one or more excipients comprising one or more of cofactors, surfactants, crowding reagents, reducing agents, oxidizing agents, denaturing agents, or enzymes. In another aspect, further comprising using centrifugation to separate dense phase separated proteins bound to the biomolecule from contaminant biomolecules. In another aspect, further comprising using centrifugation to separate phase separated proteins bound to the biomolecule from contaminant biomolecules. In another aspect, further comprising using the size of the phase separated droplets to isolate the biomolecule from contaminant species, wherein the size of the binding polypeptide bound to the biomolecule is at least 20 nm in diameter and no larger than 100 μm in diameter. In another aspect, the method comprising using flow filtration, membrane chromatography, analytical ultracentrifugation, high performance liquid chromatography, membrane chromatography, normal flow filtration, acoustic wave separation, centrifugation, counterflow centrifugation, and fast protein liquid chromatography to isolation the biomolecule-binding polypeptide complex from contaminant species on the basis of size.

Another embodiment described herein is a biomolecule comprising of at least one of a lipid, a cell, a protein, a nucleic acid, a carbohydrate or a viral particle, wherein the nucleic acid is a single stranded or double stranded DNA or RNA; the viral particle is selected from an adenovirus particle, an adeno-associated virus particle, a lentivirus particle, a retrovirus particle, a poxvirus particle, a measle virus particle, or herpesvirus particle; and the protein is selected from human albumin, monoclonal IgG antibodies, or Fc fusion antibodies.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows key parameters that influence nanoparticle fate in vivo.

FIG. 2A-C show predicted equilibrium morphologies of AB diblock polymer in bulk. FIG. 2A shows S and S′=body-centered-cubic spheres, C and C′=hexagonally packed cylinders, G and G′=bicontinuous gyroids, and L=lamellae. FIG. 2B shows theoretical phase diagram of AB diblocks predicted by the self-consistent mean-field theory, depending on volume fraction (f) of the blocks and the segregation parameter, XN, where w is the Flory-Huggins segment-segment interaction energy and N is the degree of polymerization; CPS and CPS′=closely packed spheres. FIG. 2C shows experimental phase diagram of polyisoprene-block-polystyrene copolymers, in which fA represents the volume fraction of polyisoprene, PL=perforated lamellae.

FIG. 3 shows SDS-PAGE of RLP-ELP proteins. 1. RLP20-ELP80 (SEQ ID NO: 81); 2. RLP40-ELP80 (SEQ ID NO: 84); 3. RLP60-ELP80 (SEQ ID NO: 85); 4. RLP80-ELP80 (SEQ ID NO: 87); 5. RLP100-ELP80 (SEQ ID NO: 88); 6. RLP40-ELPS80 (SEQ ID NO: 89); 7. RLP80-ELPS80 (SEQ ID NO: 91); 8. RLP40-ELPV80 (SEQ ID NO: 92); 9. RLP80-ELPV80 (SEQ ID NO: 94); 10. RLP20-ELP40 (SEQ ID NO: 80); 11. RLP40-ELP40 (SEQ ID NO: 83); 12. RLP40-ELP160 (SEQ ID NO: 82); 13. RLP80-ELP160 (SEQ ID NO: 86).

FIG. 4 shows cryo-TEM images of RLPXX-ELP80 (SEQ ID NO: 84, 85, 87) block co-polypeptides. Overall increasing the hydrophilic weight fraction by decreasing the size of the core block will shift the self-assembly from worm-like to a spherical shape. Data collected at 10 μM in 140 mM PBS. Scale bar=500 nm.

FIG. 5 shows cryo-TEM images of RLP40-ELP80 (SEQ ID NO: 84) at increasing volume fractions does not appear to affect observed assembly state. Data collected at 10, 100 and 1000 μM in 140 mM PBS respectively. Scale bar=500 nm.

FIG. 6 shows cryo-TEM images of RLPXX-ELPYY block co-polypeptides (SEQ ID NO: 80-87). Data collected at 10 μM in 140 mM PBS. Scale bar=500 nm.

FIG. 7 shows cryo-TEM images of RLPXX-ELPSYY (SEQ ID NO: 89-91) and RLPXX-ELPVYY (SEQ ID NO: 92-94) block co-polypeptides. Data collected at 10 μM in 140 mM PBS. Scale bar=500 nm.

FIG. 8 shows cryo-TEM images of RLP-ELP block co-polypeptides with varying core hydrophobicity. Scale bar=500 nm. Data collected at 1 mg·mL⁻¹ in 140 mM PBS.

FIG. 9 shows cryo-TEM images of (GRGDSP[Y:V]S)80-ELP80 (SEQ ID NO: 110) block co-polypeptide in 140 mM PBS and distilled H₂O. Data collected at 1 mg·mL⁻¹ and 15° C.

FIG. 10 shows fluorescent measurements of pyrene peaks (I1 and I3) at various concentrations of RLPXX-ELP80 (SEQ ID NO: 84, 86) block co-polypeptides in 140 mM PBS at 20° C.

FIG. 11. Cryo-TEM images of RLP40-ELP80 block co-polypeptides where the core sequence contains varying amount of Ser and Glu. Data collected at 15° C., 1 mg·mL⁻¹ in 140 mM PBS.

FIG. 12 shows a schematic of paclitaxel loading of RLP40-ELP80 (SEQ ID NO: 84) micelles and analytic procedure.

FIG. 13 shows the relative molar ratio of paclitaxel (PTX) to RLP-ELP as determined by analytical high-performance liquid chromatography. After an area for each molecule was derived using the absorption peak for each molecule (230 nm for PTX, 275 for RLP-ELP80), this peak was normalized to the extinction coefficient of the molecule and then compared to one another.

FIG. 14 shows SDS-PAGE of RLPXX-ELP80-Fn3 (SEQ ID NO: 95-97). Ladder units are in kilodaltons. Wells are labeled with the appropriate protein in the gel. All constructs have a band around 2× the molecular weight of the main band likely indicating the formation of dimers, in the presence of the gel loading buffer. Excluding this band, all materials are 295% pure.

FIG. 15 shows thermal stability of RLP-ELP-Fn3 micelles. Spherical (RLP40-ELP80-Fn3) (SEQ ID NO: 96) and worm-like micelle (RLP80-ELP80-Fn3) (SEQ ID NO: 97) stability between room temperature (20° C.) and physiological temperature (37° C.). Data collected at 10 μM in 140 mM PBS. Filtered with 0.45 μm filter.

FIG. 16 shows thermal stability of block co-polypeptide micelles. Spherical (RLP40-ELP80) (SEQ ID NO: 84) and worm-like micelle (RLP80-ELP80) (SEQ ID NO: 87) stability between room temperature (20° C.) and physiological temperature (37° C.). Data collected at 10 μM in 140 mM PBS. Filtered with 0.45 μm filter.

FIG. 17A-D show static and dynamic light scattering raw data for RLP-ELP block co-polypeptides. Plots of Rₕ vs. angle, extrapolated to 0° for reported Rₕ are show in FIG. 17A: RLP20-ELP80-Fn3, (SEQ ID NO: 95); FIG. 17B: RLP40-ELP80-Fn3-10 (SEQ ID NO: 96); and FIG. 17C: RLP80-ELP80-Fn3 (SEQ ID NO: 97). FIG. 17D-E show partial Zimm plots obtained by static light scattering; FIG. 17D: RLP40-ELP80-Fn3 (SEQ ID NO: 96); FIG. 17E: RLP80-ELP80-Fn3 (SEQ ID NO: 97).

FIG. 18A-D show cryo-TEM micrographs of RLPXX-ELP80 and RLPXX-ELP80-Fn3. FIG. 18A shows spherical micelles formed by RLP40-ELP80 (SEQ ID NO: 84). FIG. 18B shows worm-like micelles formed by RLP80-ELP80 (SEQ ID NO: 87), FIG. 18C shows spherical micelles formed by RLP40-ELP80-Fn3 (SEQ ID NO: 96). FIG. 18D shows spherical and worm-like and spherical micelles formed by RLP80-ELP80-Fn3 (SEQ ID NO: 97). All scale bars represent 200 nm. All data collected at 15° C. in 140 mM PBS at 10 μM.

FIG. 19 shows histogram of Observed Aspect Ratios of RLPXX-ELPYY-Fn3s (SEQ ID NO: 95-97). Using the data from FIG. 18, particularly the longest straight line of an individual particle and a corresponding perpendicular measurement were made to describe the aspect ratio of particles observed. Only particles that were clearly individual particles were used for the analysis (equal contrast around the particle, suggesting a camera-normal orientation). n=50.

FIG. 20 shows shape dependent avidity of RLPXX-ELP80-Fn3 (SEQ ID NO: 95-97). Multivalency increases the observed K_{D} as does increasing the aspect ratio of the micelle. Representative SPR sensor grams shown on top show a marked decrease in k_{off} between unimer, and spherical and worm-like micelles. In contrast, the kₒₙ is similar for all constructs interest. SPR sensorgram data collected in PBS at 10 μM.

FIG. 21A-B show intracellular uptake of undecorated block co-polypeptides. FIG. 21A shows representative images of cellular uptake of RLPXX-ELP80 (SEQ ID NO: 81, 84, 87) block co-polypeptides without a Fn3 domain, labeled with Alexa488 fluorophore (green) overlaid with DIC images (grey) after 2.5 hr of incubation in serum free minimal media at 10 μM. Scale bar=20 μm. FIG. 21B shows quantification of number of intracellular particles from confocal microscopy images, n>100, *=p<0.05, **=p<0.01, ***=p<0.001.

FIG. 22 shows representative images of cellular uptake of RLPXX-ELP (SEQ ID NO: 84, 87) block co-polypeptides labeled with Alexa488 fluorophore (green) overlaid with DIC images after 2.5 hr of incubation in serum free minimal media at 10 μM. Scale bar=20 μm.

FIG. 23A-D show cellular uptake of RLPXX-ELPYY-Fn3 (SEQ ID NO: 95-97) Polypeptides in αvβ3 Negative K562 Cell Line. A-D Representative images of cellular uptake of block polypeptides labeled with Alexa488 fluorophore (green) overlaid with DIC images (grey) after 2.5 hr of incubation in serum free minimal media at 10 μM. A. LM609 Antibody; B. RLP20-ELP80-Fn3 (SEQ ID NO: 95); C. RLP40-ELP80-Fn3 (SEQ ID NO: 96); D. RLP80-ELP80-Fn3 (SEQ ID NO: 97). Scale bar=20 μm

FIG. 24 shows flow cytometry data of naïve cells, LM609 antibody, RLP40-ELP80-Fn3 (SEQ ID NO: 96)—spherical micelles and RLP80-ELP80-Fn3 (SEQ ID NO: 97)—worm-like micelles.

FIG. 25 shows quantification of cellular uptake by flow cytometry. ***=p<0.001. Box indicates 25^{th} and 75^{th} percentile and bars indicate 10^{th} and 90^{th} percentile.

FIG. 26A-D show cellular uptake of RLPXX-ELPYY-Fn3 (SEQ ID NO: 96-99) polypeptides with variable aspect ratio in αvβ3 transfected cell line. A-D Representative images of cellular uptake of block polypeptides labeled with Alexa488 fluorophore (green) overlaid with DIC images (grey) after 1.5 h of incubation in serum free minimal media at 10 μM. A. RLP80-ELP80-Fn3 (SEQ ID NO: 97; B. RLP80-ELP160-Fn3 (SEQ ID NO: 98); C. RLP40-ELP80-Fn3 (SEQ ID NO: 96) D. RLP40-ELP40-Fn3 (SEQ ID NO: 99). There is a much lower level of uptake of all constructs with spherical morphologies (B, C) versus particles with elongated morphologies (A, D). Scale bar=20 μm.

FIG. 27A-B show cryo-TEM Characterization of “shape control” RLPXX-ELPYY-Fn3s. A. spherical micelles formed by RLP80-ELP160-Fn3 (SEQ ID NO: 98), B. spherical and worm-like micelles formed by RLP40-ELP80-Fn3. All data collected at 15° C. in 140 mM PBS at 10 μM.

FIG. 28 shows cellular uptake of block co-polypeptides over time. Representative confocal images of antibody (LM609), RLP20-ELP80-Fn3 (SEQ ID NO: 95), RLP40-ELP80-Fn3 (SEQ ID NO: 99) and RLP80-ELP80-Fn3 (SEQ ID NO: 97) uptake as a function of time. Scale bar=20 μm.

FIG. 29A-B show quantification of cellular uptake with image analysis. FIG. 29A shows quantification of number of intracellular particles over time. FIG. 29B shows quantification of the area of intracellular particles over time. *=p<0.05, **=p<0.01, ***=p<0.001. Error bars indicate standard deviation

FIG. 30A-D. A. [S]-40-[QHN]-40 (SEQ ID NO: 100) cryo-TEM image. Scale bar 500 nm. B. [S]-80-[QHN]-40 (SEQ ID NO: 101) cryo-TEM image. Scale bar 500 nm. C. [S]-40-[QHN]-40 (SEQ ID NO: 100) cryo-TEM image. Scale bar 200 nm. D. [S]-80-[QHN]-40 (SEQ ID NO: 101) cryo-TEM image. Scale bar 200 nm. All constructs were vitrified at 2 mg·mL⁻¹, 100% humidity, 37° C. in 140 mM PBS.

FIG. 31A-B. FIG. 31A shows UCST phase behavior of [S]-40-[QHN]-40 (SEQ ID NO: 100) and [S]-80-[QHN]-40 (SEQ ID NO: 101) block co-polypeptides as determined by UV-Vis spectrophotometry. FIG. 31B shows pH effect on UCST behavior of RLP-RLP block co-polypeptides as observed via temperature dependent DLS. Data is taken at 2 mg-mL-1 in 140 mM PBS where the UCST of both block co-polypeptides is very similar and hence a similar pH triggered UCST deflection is observed.

FIG. 32A shows critical micelle concentrations (CMCs) of [S]-40-[QHN]-40 (SEQ ID NO: 100) and [S]-80-[QHN]-40 (SEQ ID NO: 101) determined to be 3 μM and 0.4 μM respectively by a shift in I1/I13 of pyrene fluorescence. Sigmoidal fit to triplicate data is shown. CMC is determined by the inflection point of the sigmoidal fit. FIG. 32B shows full thermal characterization of [S]-40-[QHN]-40 and (SEQ ID NO: 100) [S]-80-[QHN]-40 (SEQ ID NO: 101) indicates that increasing the core block shifts the disassembly UCST phase behavior.

FIG. 33A-B show UV-Vis spectrophotometry and dynamic light scattering of [S]-40-[V]-40 (SEQ ID NO: 105) and [S]-40-[Y:3V]-40 (SEQ ID NO: 104) block co-polypeptides.

FIG. 34 shows cryo-TEM images of [S]-40-[V]-20 (SEQ ID NO: 105), [S]-40-[V]-40 (SEQ ID NO: 106), [S]-40-[V]-60 (SEQ ID NO: 107). Data collected at 15° C. in 140 mM PBS. Scale bar=500 nm.

FIG. 35 shows cryo-TEM images of [S]-40-[A]-40 (SEQ ID NO: 108), [S]-40-[V]-40(SEQ ID NO: 106), [S]-40-[I1]-40 (SEQ ID NO: 109). Data collected at 15° C. in 140 mM PBS. Scale bar=500 nm.

FIG. 36 shows a stability comparison of the two pAzF-containing sphere-forming diblock constructs investigated in this study. The constructs were mixed with the pAzF-free DB-40 diblock at different ratios, crosslinked at 7 μM and their hydrodynamic radii recorded in 7.2 M GuHCl at 700 nM using DLS. Note that both pAzF constructs failed to create stably crosslinked particles once the pAzF-per-polypeptide ratio drops below 1.

FIG. 37A-C show cryo-TEM analysis of the pAzF-containing constructs UAA5-40 and UAA4-80: FIG. 37A shows the presence of visible particles in GuHCl proved successful crosslinking for the UAA5-40 construct. Scale bars represent 100 nm. FIG. 37B shows image analysis of the core radii of the UAA5-40 particles showed significant swelling after GuHCl exposure. The particles appear smaller as only the collapsed RLP core has a high enough electron density for TEM. 100 particles were measured per condition. FIG. 37C shows the UAA4-80 construct resided as highly elongated, flexible worms after crosslinking that retained their morphology even in the presence of GuHCl. Scale bars represent 300 nm.

FIG. 38A-B show CAC determination of both sphere- and worm-forming constructs using DLS. Whereas the crosslinked samples showed stable nanoparticle readings down to the low nanomolar range—the estimated limit of detection for the DLS instrument—all other samples seemed to disassemble above that threshold. Generally, the worm-forming constructs (FIG. 38B) had lower CACs than their spherical analogues (FIG. 38A) and so do pAzF-containing constructs in comparison to analogous pAzF-free polypeptides. Note that all samples were prepared in PBS and that the error bars correspond to the standard deviation over 20 measurements.

FIG. 39A-B show SDS-PAGE gels (FIG. 39A) and protein yields (FIG. 39B) after expression and purification of all UAA5-40-K8D4-ligand constructs of this study. Note that with the exception of the TRAIL sample (pink), all lanes show bands of the targeted mass. Note also that both AHNP and TRAIL peptide ligands contain cysteine residues due to which we see faint bands corresponding to the dimers on the SDS-PAGE gel.

FIG. 40A-B show cell viability assays testing the cytotoxicity of the polybia-MPI, Tn3 and TRAIL peptide ligands. All ligands were tested on crosslinked UAA5-40 nanoparticles and were co-incubated with Colo205 (where the ligand is either Tn3 or TRAIL peptide) and K562 cells (where the ligand is Polybia-MPI) respectively over 24 hours.

FIG. 41 shows Confocal images from the cell uptake study on the breast cancer cell line SK-BR-3 using crosslinked UAA5-40-K8D4-ligand nanoparticles at a concentration of 7 μM. Apart from the Fn3 scaffold, all ligands showed significant increases in cell uptake in comparison to the unfunctionalized control. In theory, we would have expected this effect to only occur for the AHNP ligand as it targets the ErbB2 receptor on SK-BR-3 cells. Note that the brightfield contrast is extremely bad due to cell adhesion on the plate but there are around 20 cells in each of the images. Scale bars represent 30 μm.

FIG. 42A-B show confocal images of native and αvβ3-transfected K562 cells after co-incubation with AF488-tagged, crosslinked UAA5-40-K8D4-ligand nanoparticles. Scale bars represent 20 μm. FIG. 42A shows cell uptake studies above the CAC of the sphere-forming ELP/RLP diblock construct. Comparison between the native and αvβ3-transfected cell line indicates that the increased uptake observed for Fn3 and GRGDSPAS ligands was caused by integrin presentation on the cell membrane. Note that the cell uptake was not homogenous over the population which is due to previously reported variability in integrin expression levels for this cell line 30. Polybia-MPI on the other hand showed increased uptake for both cell lines but more so for the native variant. FIG. 42B shows analogous experiments at concentrations below the CAC of the ELP/RLP carrier showed that both Fn3—and GRGDSPAS—but not Polybia-MPI—constructs still had increased cell uptake compared to the unfunctionalized control. Note that the brightness of these images has been adjusted in comparison to FIG. 42A due to generally decreased uptake levels.

FIG. 43A-F show characterization of the three different UAA4-80-K8D4-ligand constructs using DLS (FIG. 43A) and TEM (FIG. 43B-E). The functionalization of the UAA4-80 construct had a substantial effect on the particle morphology after crosslinking. Though spherical structures had also been observed for the unfunctionalized UAA4-80 construct (FIG. 43E) they were only a minor side product. The functionalized constructs now however formed exclusively this kind of structure. FIG. 42F shows cryo-TEM core radius as measured via Image J. Note that all samples were crosslinked at 7 μM. All scale bars represent 200 nm.

FIG. 44A-F show the characterization of the functionalized UAA4-80 constructs after removal of the K8D4-linker. Both DLS (FIG. 44A) and cryo-TEM (FIG. 44B-E) however showed that the resulting nanoparticles after crosslinking still had a spherical morphology rather than that of elongated worms. Characterization of the UAA4-80-K8D4 construct (FIG. 44A, E, F) indicated that attachment of the linker alone nevertheless also resulted in spherical morphologies. Note that all samples were crosslinked at 7 μM. All scale bars represent 200 nm.

FIG. 45A-B show cell viability plots comparing the potency of crosslinked constructs UAA5 (FIG. 45A) and UAA4 (FIG. 45B) with and without the K8D4 linker. In addition to a strong increase in potency upon introduction of the linker, the plots also showed that of the K8D4-containing constructs, the one with the smaller UAA5-40 basis was significantly more potent.

FIG. 46A-B. FIG. 46A shows a direct comparison of analogous constructs in native and crosslinked states showed clearly that crosslinking increased the potency of the respective nanoformulations by several orders of magnitude. FIG. 46B shows a comparison of the cell survival curve with the CAC data for both pAzF-free and -containing diblocks shows that the determined EC50 values almost perfectly matched the CAC of the pAzF-free DB-40/80 constructs. Thus, particle disassembly below the CAC seems to be the limiting factor in terms of potency for loosely self-assembled nanoparticles.

FIG. 47. Comparison of the cytotoxicity of crosslinked UAA5-40 nanoparticles with different degrees of Tn3 functionalization. The nanoparticles were able to tolerate partial functionalization down to at least 50% without a major decrease in potency FIG. 48A-C. FIG. 48A shows flow cytometry data for the two K562 cell lines used in this study after 90 minutes of co-incubation with either PBS or 350 nM of an anti-αvβ3 antibody. Note that for the transfected cell line, we observe a secondary subpopulation with significantly increased fluorescence. This subpopulation accounts for 12.6% of all analyzed cells. FIG. 48B-C. Based on the observation in A we decided to solely focus on the most strongly fluorescent 10% of the whole cell population through which the two cell lines can be differentiated more clearly in the boxplot diagrams. In the “full range” diagram (FIG. 48B), the boxes represent the 25th and 75th percentile and the bars the 10th and 90th percentile. In the “top 10 percent” diagram (FIG. 48C), they represent the 93^{rd}/97^{th} and 91^{st}/99^{th} percentiles respectively.

FIG. 49A-B show flow cytometry data for cell uptake experiments comparing the two different K562 cell lines of this study of UAA5 (FIG. 49A) and UAA4 (FIG. 49B). All cells were co-incubated with crosslinked AF488-tagged nanoparticles for 90 minutes. Only the particles carrying the Fn3 and GRGDSPAS ligands showed selective uptake for the αvβ3-displaying cell line. The boxes in the boxplot diagrams represent the 93^{rd} and 97^{th} percentile, the bars the 91^{st} and 99^{th} percentile.

FIG. 50A-B show flow cytometry data for the multivalency experiments on the αvβ3-transfected K562 cells. For both diblock architectures, crosslinking significantly increased cell uptake of Fn3- and GRGDSPAS-decorated nanoparticles in the sub-CAC regime. Note that the chosen concentration for the UAA4-80 construct was a compromise between its CAC (around 30-50 nM) and the limit of detection of the assay (around 10 nM). Thus, the improvements upon crosslinking were not quite as profound for the UAA4-80 (B) constructs as they were for the UAA5-40 (A) diblocks. The boxes in the boxplot diagrams represent the 93^{rd} and 97^{th} percentile, the bars the 91st and 99th percentile.

FIG. 51A-C show SPR analysis of the αvβ3 integrin binding of the UAA5-40 diblock constructs. FIG. 51A. In the crosslinked state, the Fn3- and GRGDSPAS-functionalized particles showed very high binding affinities to αvβ3 integrin. As a comparison: Dzuricky et al.'s native Fn3 constructs had a reported K_{D} of 79 nM 30. FIG. 51B. In the native state, the GRGDSPAS construct showed no binding at concentrations below the CAC. For their native Fn3 analogues on the other hand, binding was observed though at lower levels than for the crosslinked nanoparticles. FIG. 51C. At concentrations above the CAC, native and crosslinked constructs showed comparable binding affinities to αvβ3 integrin. Note that the vertical dotted line represents the point at which the buffer is exchanged.

FIG. 52A-C show SPR characterization of the integrin-targeting UAA4-80 constructs. FIG. 52A. The GRGDSPAS-functionalized construct seemed to have a sharp cut-off for binding to αvβ3 integrin as the SPR signal rapidly collapsed upon dilution below 150 nM. FIG. 52B. For the Fn3-functionalized construct, the SPR data is even more confusing as it showed good binding at 68 nM but none at concentrations both above and below that value. FIG. 52C. Thus, the only K_{D} value that could be calculated was the one for the crosslinked GRGDSPAS construct in a narrow concentration range around 170 nM. Note that the vertical dotted line marks the point at which the buffer is exchanged.

FIG. 53A-B show SPR characterization of the DR5-targeting UAA5-40 constructs. The comparison of the SPR data in FIG. 53A and FIG. 53B showed that the binding affinity of the Tn3-ligand seemed to only mildly benefit from multivalent display compared to the integrin-targeting constructs in FIG. 52. This then indicated that the requirement for multivalency for Tn3 action mainly stemmed from downstream effects after binding of the receptor and not from DR5-binding itself. Note that the vertical dotted line represents the point at which the buffer is exchanged during the SPR experiment.

FIG. 54 shows an SDS-PAGE image of the capture and release of antibody therapeutics from cell culture harvest material. Diblock materials elute a cleaner mAb product than single chain ELP unimer. Comparison of eluted final product is between wells 2-11, 4-13, and 6-15.

FIG. 55. Example purity data producing proteins that contain un-natural amino acids that can then be crosslinked into nanoparticle structures. This method is applicable to a variety of ligand sizes and architectures and can be produced at high purity using a simple purification scheme described herein.

FIG. 56 shows the cell uptake of crosslinked polypeptides scaffold with various protein domains on the corona of the nanoparticle Proteins are labeled with a green fluorescent molecule for visualization. In an engineered cell line that contains the αvβ3 receptor on the cells surface, the Fn3 and GRGDSPAS ligands that both have specificity for the receptor are able to be internalized at both concentrations tested (70 nM and 7 μM). The Polybia-MPI is only internalized at the higher concentration due to different kinetics of the ligand. This demonstrates that multivalent rapid cell internalization is achievable.

FIG. 57 shows the incubation of a mAb with a binding polypeptide that is fused to a segment of protein A (ZD). Using salt, the phase separation of the binding polypeptide-mAb complex is trigged and separated by centrifugation. This solution forms two phases, the protein rich pellet that contains the mAb-binding polypeptide complex in the precipitate and the capture supernatant (SN). The pellet is resuspended in an elution buffer that dissociates the mAb from the binding polypeptide. The solution is centrifuged again, precipitating the binding polypeptide, separate from the mAb, which remains suspended in the elution SN. In this experiment, several mAb proteins (mAb1, 2, 3) were bound and eluted.

### DETAILED DESCRIPTION

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the specification of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “about” as used herein as applied to one or more values of interest, refers to a value that is similar to a stated reference value. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Affinity” refers to the binding strength of a binding polypeptide to its target (i.e., binding partner).

“Agonist” refers to an entity that binds to a receptor and activates the receptor to produce a biological response. An ‘antagonist’ blocks or inhibits the action or signaling of the agonist. An ‘inverse agonist’ causes an action opposite to that of the agonist. The activities of agonists, antagonists, and inverse agonists may be determined in vitro, in situ, in vivo, or a combination thereof.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

As used herein, the term “biomarker” refers to a naturally occurring biological molecule present in a subject at varying concentrations that is useful in identifying and/or classifying a disease or a condition. The biomarker can include genes, proteins, polynucleotides, nucleic acids, ribonucleic acids, polypeptides, or other biological molecules used as an indicator or marker for disease. In some embodiments, the biomarker comprises a disease marker. For example, the biomarker can be a gene that is upregulated or downregulated in a subject that has a disease. As another example, the biomarker can be a polypeptide whose level is increased or decreased in a subject that has a disease or risk of developing a disease. In some embodiments, the biomarker comprises a small molecule. In some embodiments, the biomarker comprises a polypeptide.

The terms “control,” “reference level,” and “reference” are used herein interchangeably. The reference level may be a predetermined value or range, which is employed as a benchmark against which to assess the measured result. ‘Control group’ as used herein refers to a group of control subjects. The predetermined level may be a cutoff value from a control group. The predetermined level may be an average from a control group. Cutoff values (or predetermined cutoff values) may be determined by Adaptive Index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, e.g., to determine the performance of each marker in identifying a patient having CRC. A description of ROC analysis is provided in P. J. Heagerty et al. (Biometrics 2000, 56, 337-44), the disclosure of which is hereby incorporated by reference in its entirety. Alternatively, cutoff values may be determined by a quartile analysis of biological samples of a patient group. For example, a cutoff value may be determined by selecting a value that corresponds to any value in the 25^{th}-75^{th} percentile range, preferably a value that corresponds to the 25^{th} percentile, the 50^{th} percentile or the 75^{th} percentile, and more preferably the 75^{th} percentile. Such statistical analyses may be performed using any method known in the art and can be implemented through any number of commercially available software packages (e.g., from Analyse-it Software Ltd., Leeds, UK; StataCorp LP, College Station, Tex.; SAS Institute Inc., Cary, N.C.). The healthy or normal levels or ranges for a target or for a protein activity may be defined in accordance with standard practice.

The term “expression vector” indicates a plasmid, a virus, or another medium, known in the art, into which a nucleic acid sequence for encoding a desired protein can be inserted or introduced.

The term “host cell” is a cell that is susceptible to transformation, transfection, transduction, conjugation, and the like with a nucleic acid construct or expression vector. Host cells can be derived from plants, bacteria, yeast, fungi, insects, animals, etc. In some embodiments, the host cell includes Escherichia coli.

“Polymer” as used herein is intended to encompass a homopolymer, heteropolymer, block polymer, co-polymer, ter-polymer, etc., and blends, combinations and mixtures thereof.

Examples of polymers include, but are not limited to, functionalized polymers, such as a polymer comprising 5-vinyltetrazole monomer units and having a molecular weight distribution less than 2.0. The polymer may be or contain one or more of a star block copolymer, a linear polymer, a branched polymer, a hyperbranched polymer, a dendritic polymer, a comb polymer, a graft polymer, a brush polymer, a bottle-brush copolymer and a crosslinked structure, such as a block copolymer comprising a block of 5-vinyltetrazole monomer units. Polymers include, without limitation, polyesters, poly(meth)acrylamides, poly(meth)acrylates, polyethers, polystyrenes, polynorbornenes and monomers that have unsaturated bonds. For example, amphiphilic comb polymers are described in U.S. Patent Application Publication No. US 2007/0087114 and in U.S. Pat. No. 6,207,749 to Mayes et al., the disclosure of each of which is herein incorporated by reference in its entirety. The amphiphilic comb-type polymers may be present in the form of copolymers, containing a backbone formed of a hydrophobic, water-insoluble polymer and side chains formed of short, hydrophilic non-cell binding polymers. Examples of other polymers include, but are not limited to, polyalkylenes such as polyethylene and polypropylene; polychloroprene; polyvinyl ethers; such as polyvinyl acetate); polyvinyl halides such as polyvinyl chloride); polysiloxanes; polystyrenes; polyurethanes; polyacrylates; such as poly(methyl (meth)acrylate), poly(ethyl (meth)acrylate), poly(n-butyl(meth)acrylate), poly(isobutyl (meth)acrylate), poly(tert-butyl (meth)acrylate), poly(hexyl(meth)acrylate), poly(isodecyl (meth)acrylate), poly(lauryl (meth)acrylate), poly(phenyl (meth)acrylate), poly(methyl acrylate), poly(isopropyl acrylate), poly(isobutyl acrylate), and poly(octadecyl acrylate); polyacrylamides such as poly(acrylamide), poly(methacrylamide), poly(ethyl acrylamide), poly(ethyl methacrylamide), poly(N-isopropyl acrylamide), poly(n, iso, and tert-butyl acrylamide); and copolymers and mixtures thereof. These polymers may include useful derivatives, including polymers having substitutions, additions of chemical groups, for example, alkyl groups, alkylene groups, hydroxylations, oxidations, and other modifications routinely made by those skilled in the art. The polymers may include zwitterionic polymers such as, for example, polyphosphorycholine, polycarboxybetaine, and polysulfobetaine. The polymers may have side chains of betaine, carboxybetaine, sulfobetaine, oligoethylene glycol (OEG), sarcosine, or polyethyleneglycol (PEG). For example, poly(oligoethyleneglycol methacrylate) (poly(OEGMA)) may be used. Poly(OEGMA) may be hydrophilic, water-soluble, non-fouling, non-toxic and non-immunogenic due to the OEG side chains.

“Polynucleotide” as used herein can be single stranded or double stranded or can contain portions of both double stranded and single stranded sequence. The polynucleotide can be nucleic acid, natural or synthetic, DNA, genomic DNA, cDNA, RNA, or a hybrid, where the polynucleotide can contain combinations of deoxyribo- and ribo-nucleotides, and combinations of bases including uracil, adenine, thymine, cytosine, guanine, inosine, xanthine hypoxanthine, isocytosine, and isoguanine. Polynucleotides can be obtained by chemical synthesis methods or by recombinant methods.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide,” “protein,” and “peptide” are used interchangeably herein. “Primary structure” refers to the amino acid sequence of a particular peptide. “Secondary structure” refers to locally ordered, three-dimensional structures within a polypeptide. These structures are commonly known as domains, e.g., enzymatic domains, extracellular domains, transmembrane domains, pore domains, and cytoplasmic tail domains. Domains are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three-dimensional structure of a polypeptide monomer. “Quaternary structure” refers to the three-dimensional structure formed by the noncovalent association of independent tertiary units.

“Reporter,” “reporter group,” “label,” and “detectable label” are used interchangeably herein. The reporter is capable of generating a detectable signal. The label can produce a signal that is detectable by visual or instrumental means. A variety of reporter groups can be used, differing in the physical nature of signal transduction (e.g., fluorescence, electrochemical, nuclear magnetic resonance (NMR), and electron paramagnetic resonance (EPR)) and in the chemical nature of the reporter group. Various reporters include signal-producing substances, such as chromagens, fluorescent compounds, chemiluminescent compounds, radioactive compounds, and the like. In some embodiments, the reporter comprises a radiolabel. Reporters may include moieties that produce light, e.g., acridinium compounds, and moieties that produce fluorescence, e.g., fluorescein. In some embodiments, the signal from the reporter is a fluorescent signal. The reporter may comprise a fluorophore. Examples of fluorophores include, but are not limited to, acrylodan (6-acryloyl-2-dimethylaminonaphthalene), badan (6-bromo-acetyl-2-dimethylamino-naphthalene), rhodamine, naphthalene, danzyl aziridine, 4-[N-[(2-iodoacetoxy)ethyl]-N-methylamino]-7-nitrobenz-2-oxa-1,3-diazole ester (IANBDE), 4-[N-[(2-iodoacetoxy)ethyl]-N-methylamino-7-nitrobenz-2-oxa-1,3-diazole (IANBDA), fluorescein, dipyrrometheneboron difluoride (BODIPY), 4-nitrobenzo[c][1,2,5]oxadiazole (NBD), Alexa fluorescent dyes, and derivatives thereof. Fluorescein derivatives may include, for example, 5-fluorescein, 6-carboxyfluorescein, 3′6-carboxyfluorescein, 5(6)-carboxyfluorescein, 6-hexachlorofluorescein, 6-tetrachlorofluorescein, fluorescein, and isothiocyanate.

“Sample” or “test sample” as used herein can mean any sample in which the presence and/or level of a target is to be detected or determined. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

The term “sensitivity” as used herein refers to the number of true positives divided by the number of true positives plus the number of false negatives, where sensitivity (“sens”) may be within the range of 0<sens<1. Ideally, method embodiments herein have the number of false negatives equaling zero or close to equaling zero, so that no subject is wrongly identified as not having a disease when they indeed have the disease. Conversely, an assessment often is made of the ability of a prediction algorithm to classify negatives correctly, a complementary measurement to sensitivity.

The term ‘specificity’ as used herein refers to the number of true negatives divided by the number of true negatives plus the number of false positives, where specificity (“spec”) may be within the range of 0<spec<1. Ideally, the methods described herein have the number of false positives equaling zero or close to equaling zero, so that no subject is wrongly identified as having a disease when they do not in fact have disease. Hence, a method that has both sensitivity and specificity equaling one, or 100%, is preferred.

By “specifically binds,” it is generally meant that a polypeptide binds to a target when it binds to that target more readily than it would bind to a random, unrelated target.

“Subject” as used herein can mean a mammal that wants or is in need of the herein described nanoparticles comprising one or more fusion proteins. The subject may be a human or a non-human animal. The subject may be a mammal. The mammal may be a primate or a non-primate. The mammal can be a primate such as a human; a non-primate such as, for example, dog, cat, horse, cow, pig, mouse, rat, camel, llama, goat, rabbit, sheep, hamster, and guinea pig; or non-human primate such as, for example, monkey, chimpanzee, gorilla, orangutan, and gibbon. The subject may be of any age or stage of development, such as, for example, an adult, an adolescent, or an infant.

“Transition” or “phase transition” refers to the aggregation of the thermally responsive polypeptides. Phase transition occurs sharply and reversibly at a specific temperature called the lower critical solution temperature (LCST) or the inverse transition temperature T\hat{ }. Below the transition temperature, the thermally responsive polypeptide (or a polypeptide comprising a thermally responsive polypeptide) is highly soluble. Upon heating past the transition temperature, the thermally responsive polypeptides hydrophobically collapse and aggregate, forming a separate, gel-like phase. “Inverse transition cycling” refers to a protein purification method for thermally responsive polypeptides (or a polypeptide comprising a thermally responsive polypeptide). The protein purification method may involve the use of thermally responsive polypeptide's reversible phase transition behavior to cycle the solution through soluble and insoluble phases, thereby removing contaminants.

“Treatment” or “treating,” when referring to protection of a subject from a disease, means preventing, suppressing, repressing, ameliorating, or eliminating the disease. Preventing the disease involves administering a composition of the present invention to a subject prior to onset of the disease. Suppressing the disease involves administering a composition of the present invention to a subject after induction of the disease but before its clinical appearance. Repressing or ameliorating the disease involves administering a composition of the present invention to a subject after clinical appearance of the disease.

“Substantially identical” can mean that a first and second amino acid sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or greater number of amino acids.

“Valency” as used herein refers to the potential binding units or binding sites. The term “multivalent” refers to multiple potential binding units. The terms ‘multimeric’ and “multivalent” are used interchangeably herein.

“Variant” used herein with respect to a polynucleotide means (i) a portion or fragment of a referenced nucleotide sequence; (ii) the complement of a referenced nucleotide sequence or portion thereof; (iii) a polynucleotide that is substantially identical to a referenced polynucleotide or the complement thereof; or (iv) a polynucleotide that hybridizes under stringent conditions to the referenced polynucleotide, complement thereof, or a sequences substantially identical thereto.

A “variant” can further be defined as a peptide or polypeptide that differs in amino acid sequence by the insertion, deletion, or conservative substitution of amino acids, but retain at least one biological activity. Representative examples of “biological activity” include the ability to be bound by a specific antibody or polypeptide or to promote an immune response. Variant can mean a substantially identical sequence. Variant can mean a functional fragment thereof. Variant can also mean multiple copies of a polypeptide. The multiple copies can be in tandem or separated by a linker. Variant can also mean a polypeptide with an amino acid sequence that is substantially identical to a referenced polypeptide with an amino acid sequence that retains at least one biological activity. A conservative substitution of an amino acid, i.e., replacing an amino acid with a different amino acid of similar properties (e.g., hydrophilicity, degree, and distribution of charged regions) is recognized in the art as typically involving a minor change. These minor changes can be identified, in part, by considering the hydropathic index of amino acids. See Kyte et al., J. Mol. Biol. 1982, 757, 105-132. The hydropathic index of an amino acid is based on a consideration of its hydrophobicity and charge. It is known in the art that amino acids of similar hydropathic indexes can be substituted and retain protein function. In one aspect, amino acids having hydropathic indices of t 2 are substituted. The hydrophobicity of amino acids can also be used to reveal substitutions that would result in polypeptides retaining biological function. A consideration of the hydrophilicity of amino acids in the context of a polypeptide permits calculation of the greatest local average hydrophilicity of that polypeptide, a useful measure that has been reported to correlate well with antigenicity and immunogenicity, as discussed in U.S. Pat. No. 4,554,101, which is incorporated herein by reference. Substitution of amino acids having similar hydrophilicity values can result in polypeptides retaining biological activity, for example immunogenicity, as is understood in the art. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant can be a polynucleotide sequence that is substantially identical over the full length of the full gene sequence or a fragment thereof. The polynucleotide sequence can be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof.

A variant can be an amino acid sequence that is substantially identical over the full length of the amino acid sequence or fragment thereof. The amino acid sequence can be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

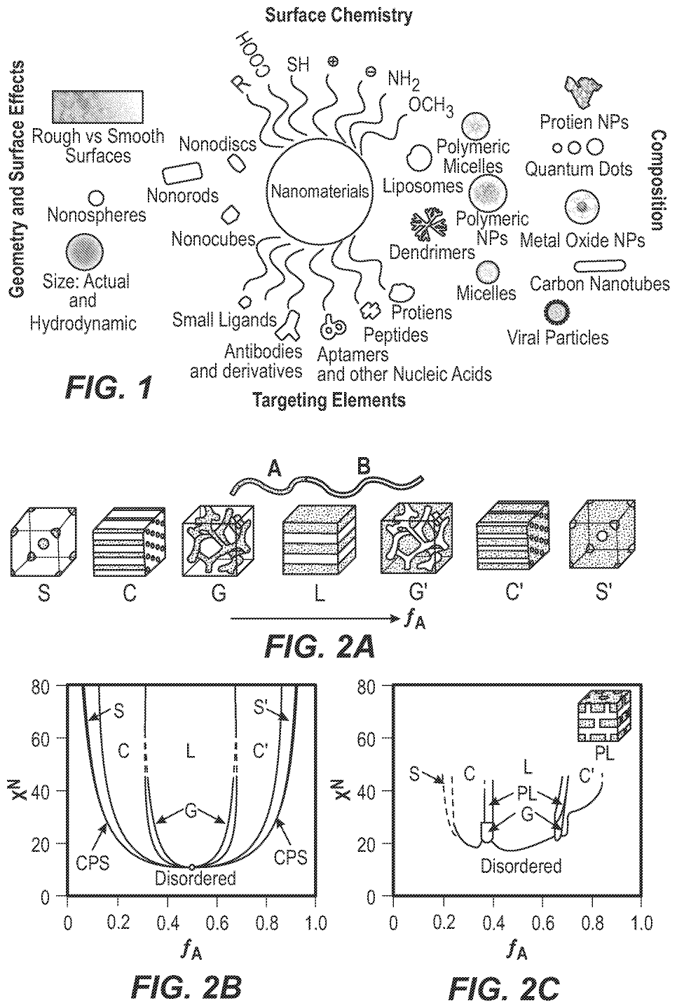

In recent years, due to advances in polymer synthesis technology, the ability to investigate specific biophysical properties of nanomaterials has received much research attention. Almost every aspect of a nanoparticle's design has been tested for improved efficacy in along some dimension (biocompatibility, pK, tissue extravasation etc). A brief list of the parameters is highlighted in FIG. 1 and a brief summary of optimal design specifications is included.

Size: Large particles (>1 μm) are internalized by macrophages, neutrophils, dendritic cells. Smaller than 1 μm, they are internalized via pinocytosis or receptor mediated endocytosis. Particles in the 40-50 nm range exhibit maximum uptake. Particles between 10-100 nm are typical size ranges for optimization of biodistribution and clearance. Smaller than 5.5 nm are rapidly cleared by the kidneys. It is thought that corona chain curvature and conformation is particularly crucial for determining in vivo fate.

Shape: Rod-like designs are more readily taken into cells than spherical counterparts. Non-spherical particles appear to have longer circulation times compared to spherical counterparts.

Surface chemistry: Charged nanoparticles have shorter blood circulation times and highly nonspecific cellular uptake. This can be readily tuned in most synthetic polymer systems, but neutral charge is generally the best for most applications.

Corona Hydrophobicity: Block copolymers with increased corona hydrophobicity are more easily taken up by cells but also have higher levels of opsonization. For in vivo applications, the optimal formulation will vary, especially when targeted therapies are concerned.

Core stability: Micelle half-life can be controlled via core stability as measured via pyrene I1/I3 fluorescence. Other studies have demonstrated that crosslinking polymeric micelle cores can also increase the observed half-life in vivo.

Particle rigidity: Deformable structures can last up to 30 times longer in circulation than rigid counterparts.

Targeting/stimuli responsive elements: Targeting has generally improved nanocarriers compared to a non-targeted system. However, the incorporation of targeting ligands or environmentally sensitive moieties often alters the surface charge, morphology or both. One study has examined the effect of ligand density on tumor targeting and did find that an optimal ratio exists for that particular cancer phenotype. This result indicates that an opsonization versus targeting tradeoff exists and must be accounted for when introducing targeting ligands.

With synthetic polymer systems, the most challenging of these parameters to control are the targeting/responsive elements and geometry. These design parameters can often not be controlled with one pot synthesis and therefore multiple step construction is required. Multistep processes almost inevitably introduce polydispersity/heterogeneity, which can cloud the conclusions made about a particular design choice. In fact, a recent study demonstrated that a 10-20 nm deviation could significantly affect nanoparticle behavior in the body. Thus, despite all the advances in polymer micelle synthesis, it is still difficult to design optimal micelle carriers. In an ideal scenario, one would a priori be able to incorporate optimal design elements for a particular application at the design stage.

Controlling morphology of block copolymers: Micro-phase separation of diblock copolymers depends on three parameters: volume fraction of both blocks combined, total degree of polymerization and the Flory-Huggins parameters (x). The chi parameter specifies the miscibility of both the blocks, or in an amphiphilic block copolymer case, the immiscibility. The chi parameter is also a function of temperature. For a system consisting of just the block copolymer, the chi parameter contains interaction energies between blocks A-B, A-A, B-B. Increasing the temperature or decreasing chi, compatibility between the blocks improves combinatorial entropy increases and copolymers undergo an order to disorder transition.

$${χ}_{A B} = ( \frac{z}{{k}_{b} T} ) [ {ε}_{A B} - \frac{1}{2} ( {ε}_{AA} + {ε}_{BB} ) ]$$

The number of chi parameters jumps to six once water is introduced into the system since each block can interact with itself, the other block, and water. However, controlling morphology in aqueous solution can be simplified to a function of three polymer primary variables-interfacial energy between the blocks (enthalpic), chain stretching of the core (entropic) and chain repulsion in the corona. A balance between repulsive corona-corona interactions and conformational entropy penalty for extending the chains determines the actual conformations of the corona chains. It is important to note that this balance is affected by the self-assembled morphology. The core block extension is also affected by morphology. Core chains are most extended in a spherical morphology and most compact in a rod-like morphology.

Upon the formation of microstructure, the blocks attempt to minimize the total interfacial energy of the system. During this process, they sacrifice the entropic gains of forming single chains, to prevent from paying an even larger penalty of hydrophobic-water interactions. This lowers the total free energy of the system. Increasing the size of the core block (A) the corona volume fraction of the total length of the chain decreases. As a result, less curvature is observed at the interface of the polymer chain.

Key parameters of interest are the hydrophilicity of the corona-forming block, the hydrophilicity of the core block, the overall length of the co-polypeptide and the ratio of the two blocks. Parameters of evaluation are the size, morphology, stability, and thermoresponsive behavior. All measurements were made in 140 mM NaCl, 10 mM phosphate buffer, 3 mM KCl, pH 7.4 unless otherwise specified. Size was evaluated by the hydrodynamic radios (Rₕ) and radius of gyration (R_{g}) by dynamic light scattering (DLS) and static light scattering (SLS) respectively. R_{g} and Rₕ can be combined to yield the shape factor ρ=R_{g}/Rₕ, which provides a rough indication of the morphology of the scatterer. A shape factor of 1.505 suggests a Gaussian polymer chain, 1.0 suggests a hollow sphere or vesicle, and 0.775 suggests a solid sphere. For an elongated scatterer, the shape factor depends upon the aspect ratio. A combination of temperature dependent turbidity and DLS was utilized to determine the phase behavior of the block co-polypeptides. Cryogenic transmission electron microscopy (Cryo-TEM) was utilized to evaluate the morphology and provide crucial insight into the hydration of the core/corona chains. Stability of the assembled nanostructure was determined by a shift in the I1/I3 fluorescent bands of pyrene as described previously.

One of the targeting domains chosen for the second portion of this study is the 10th, type III domain from human fibronectin (Fn3) that targets the human αvβ3 integrin, a receptor that is upregulated in the endothelium of many tumors and is also overexpressed on several tumor cells such as glioblastoma, renal cell carcinoma, ovarian carcinoma and breast cancer metastases. We chose an Fn3 variant that binds the αvβ3 integrin with low affinity (K_{D}>1×10⁻⁷ M), and we have previously shown that the Fn3 domain can be expressed in E. coli as a fusion to repetitive polypeptides such as ELPs. The low affinity of the parent Fn3 domain is important as, multivalent presentation could amplify its avidity, which may not be possible with ligands that possess intrinsically high affinity, so that we could test for the effect of self-assembly and multivalency on binding avidity and cellular uptake.

### Fusion Protein

The term “fusion protein” as described herein at least one unstructured polypeptide and at least one binding polypeptide. The fusion protein may optionally include at least one linker.

In some embodiments, the fusion protein includes more than one unstructured polypeptide. The fusion protein may include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 unstructured polypeptides. The fusion protein may include less than 30, less than 25, or less than 20 unstructured polypeptides. The fusion protein may include between 1 and 30, between 1 and 20, or between 1 and 10 unstructured polypeptides. In such embodiments, the unstructured polypeptides may be the same or different from one another. In some embodiments, the fusion protein includes more than one unstructured polypeptide positioned in tandem to one another. In one embodiment, the fusion protein comprises a di-block of two unstructured polypeptides with various repeats of the two individual unstructured polypeptides.

In some embodiments, the fusion protein includes more than one binding polypeptide. The fusion protein may include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 binding polypeptides. The fusion protein may include less than 30, less than 25, less than 20, less than 10, or less than 5 binding polypeptides. The fusion protein may include between 1 and 30, between 1 and 20, or between 1 and 10 binding polypeptides. In such embodiments, the binding polypeptides may be the same or different from one another. In some embodiments, the fusion protein includes more than one binding polypeptide positioned in tandem to one another. In some embodiments, the fusion protein includes 2 to 6 binding polypeptides. In some embodiments, the fusion protein includes two binding polypeptides. In some embodiments, the fusion protein includes three binding polypeptides. In some embodiments, the fusion protein includes four binding polypeptides. In some embodiments, the fusion protein includes five binding polypeptides. In some embodiments, the fusion protein includes six binding polypeptides.

In some embodiments, the fusion protein may be arranged as a modular linear polypeptide. For example, the modular linear polypeptide may be arranged in one of the following structures:
[UPX]ₙ-[UPY]ₘ-[BP]ₚ;
[UPY]ₘ-[UPX]ₙ-[BP]ₚ;
[BP]ₚ-[UPX]ₙ-[UPY]ₘ;
[BP]ₚ-[UPY]ₘ-[UPX]ₙ;
[UPX]ₙ-[BP]ₚ-[UPY]ₘ;
[UPY]ₘ-[BP]ₚ-[UPX]ₙ;
[BP]ₚ-[UPX]ₙ-[UPY]ₘ-[BP]ₚ;
[BP]ₚ-[UPY]ₘ-[UPX]ₙ-[BP]ₚ;
[BP]ₚ-[UPX]ₙ-[BP]ₚ-[UPY]ₘ-[BP]ₚ;
[BP]ₚ-[UPY]ₘ-[BP]ₚ-[UPX]ₙ-[BP]ₚ;
   where UPX refers to unstructured protein X, UPY refers to unstructured protein Y, BP refers to binding polypeptide; where unstructured polypeptide X is a different unstructured polypeptide than unstructured polypeptide Y and where n, m, and p are each independently an integer greater than or equal to 1, and “-” represents a bond or a linker moiety. In some embodiments, n is an integer from 20 to 200. In one aspect, n is 40 to 200. In some embodiments, m is an integer from 20 to 200. In one aspect, n is 40 to 200. In some embodiments, p is an integer less than or equal to 10. In some embodiments, p is an integer equal to 1, 2, 3, 4, 5, 6, 7, 8, 9, or 10. In some embodiments, at least one binding polypeptide is positioned N-terminal to at least one unstructured polypeptide. In some embodiments, at least one binding polypeptide is positioned C-terminal to at least one unstructured polypeptide. Other iterations of the motifs shown above are contemplated and are within the scope of this disclosure.

The fusion protein may be expressed recombinantly in a host cell according to one of ordinary skill in the art. The fusion protein may be purified by any means known to one of skill in the art. For example, the fusion protein may be purified using chromatography, such as liquid chromatography, size exclusion chromatography, or affinity chromatography, or a combination thereof. In some embodiments, the fusion protein is purified without chromatography. In some embodiments, the fusion protein is purified using inverse transition cycling.

In one embodiment, the fusion protein comprises an COREₙ-CORONAₘ di-block linked to a binding polypeptide, where n is 20-200 repeats and m is 40-200 repeats. In one aspect, the binding polypeptide comprises Fn3, Tn3, alpha helical Z domain of Staphylococcus aureus protein A, one or more targeting peptides, anti-EGFR binding protein, DARPINS, knottins, or scFvs

In one embodiment, the fusion protein comprises an RLPₙ-ELPₘ di-block linked to a binding polypeptide, where n is 20-200 repeats and m is 40-200 repeats. In one aspect, the binding polypeptide comprises Fn3, Tn3, alpha helical Z domain of Staphylococcus aureus protein A, one or more targeting peptides, anti-EGFR binding protein, DARPINS, knottins, or scFvs

### Unstructured Polypeptide

The unstructured polypeptide may comprise any polypeptide that has minimal or no secondary structure as observed by CD, being soluble at a temperature below its lower critical solution temperature (LCST) and/or at a temperature above its upper critical solution temperature (UCST), and comprising a repeated amino acid sequence. LCST is the temperature below which the polypeptide is miscible. UCST is the temperature above which the polypeptide is miscible. In some embodiments, the unstructured polypeptide has only UCST behavior. In some embodiments, the unstructured polypeptide has only LCST behavior. In some embodiments, the unstructured polypeptide has both UCST and LCST behavior. The unstructured polypeptide may comprise a repeated sequence of amino acids. The unstructured polypeptide may have a LCST between about 0° C. and about 100° C., between about 10° C. and about 50° C., or between about 20° C. and about 42° C. The unstructured polypeptide may have a UCST between about 0° C. and about 100° C., between about 10° C. and about 50° C., or between about 20° C. and about 42° C. In some embodiments, the unstructured polypeptide has a transition temperature between room temperature (about 25° C.) and body temperature (about 37° C.). In some embodiments, a fusion protein comprising one or more thermally responsive polypeptides has a transition temperature between room temperature (about 25° C.) and body temperature (about 37° C.). In some embodiments, the unstructured polypeptide has no LCST or UCST behavior. The unstructured polypeptide may have its LCST or UCST below body temperature or above body temperature at the concentration at which the nanoparticle comprising one or more fusion proteins is administered to a subject.

In some embodiments, the unstructured polypeptide comprises one or more thermally responsive polypeptides. Thermally responsive polypeptides may include, for example, elastin-like polypeptides (ELP) and resilin-like protein (RLP).

In some embodiment, the unstructured polypeptide comprises a plurality of unstructured polypeptides. In one aspect, the unstructured polypeptide comprises a di-block of two or more unstructured polypeptides. In one aspect, the unstructured polypeptides comprise a di-block of a resilin-like protein (RLP) and an elastin-like polypeptide (ELP).

In one embodiment, the unstructured polypeptide comprises one or more core polypeptides. In one aspect, the core polypeptide is a resilin-like polypeptide (RLP). RLPs are derived from arthropod Rec1-resilin. Rec1-resilin is environmentally responsive and exhibits a dual phase transition behavior. The thermally responsive RLPs can have LCST and UCST (Li et. al, Macromol. Rapid Commun. 2015, 36, 90-95.) Additional examples of suitable thermally responsive polypeptides are described in U.S. Patent Application Publication Nos. US 2012/0121709, and US 2015/0112022, each of which is incorporated herein by reference. In one embodiment, the RLP polypeptide comprises the sequence QYPSDGRG (SEQ ID NO: 1). The unstructured polypeptide may comprise an amino acid sequence comprising (QYPSDGRG)ₙ, where n is 20-200. In some embodiments, n is 20, 30, 40, 50, 60, 70, 80, 90, 100, 110, 120, 130, 140, 150, 160, 170, 180, 190, 200, 210, 220, 230, 240, 250, 260, 270, 280, 290, or 300. In some embodiments, n may be less than 500, less than 400, less than 300, less than 200, or less than 100. In some embodiments, n may be between 1 and 500, between 1 and 400, between 1 and 300, or between 1 and 200. In some embodiments, n is 20, 40, 60, 80, 100, 120, 160, 180, or 200. In one aspect, n is 20 to 200 repeats of RLP. RLP may be expressed recombinantly.

In another embodiment, the unstructured polypeptide comprises one or more corona polypeptides. In one aspect, the corona polypeptide comprises an elastin-like polypeptides (ELP). Elastin-like polypeptides (ELP) refers to a polypeptide comprising the sequence VPG[A:G]G (SEQ ID NO: 8). The unstructured polypeptide may comprise an amino acid sequence consisting of (VPG[A:G]G)ₙ, where n is 40-200. In some embodiments, n is 20, 30, 40, 50, 60, 70, 80, 90, 100, 110, 120, 130, 140, 150, 160, 170, 180, 190, 200, 210, 220, 230, 240, 250, 260, 270, 280, 290, or 300. In some embodiments, n may be less than 500, less than 400, less than 300, less than 200, or less than 100. In some embodiments, n may be between 1 and 500, between 40 and 400, between 1 and 300, or between 40 and 200. In some embodiments, n is 20, 40, 60, 80, 100, 120, 160, 180, or 200. In one aspect, n is 40 to 200 repeats of ELP. ELP may be expressed recombinantly.

The unstructured polypeptide(s) may further include additional amino acids at the C-terminal or N-terminal end of the ELP or RLP motif. These amino acids surrounding the motif may also be part of the overall repeated motif. The amino acids that surround the motif may balance the overall hydrophobicity and/or charge to control the LCST or UCST behavior of the unstructured polypeptide.

In one embodiment, the unstructured polypeptide comprises RLPₙ-ELPₘ, where n is 20-200 repeats and m is 40-200 repeats. In one aspect, the unstructured polypeptide comprises RLP₄₀-ELP₄₀ (SEQ ID NO: 83); RLP₄₀-ELP₈₀ (SEQ ID NO: 84); RLP₈₀-ELP₈₀ (SEQ ID NO: 87); or RLP₈₀-ELPs₁₆₀ (SEQ ID NO: 86).

Thermally responsive polypeptides, for example, ELP and RLP, may have a phase transition. The thermally responsive polypeptide may impart a phase transition characteristic to the unstructured polypeptide or fusion protein. “Phase transition” or “transition” may refer to the aggregation of the thermally responsive polypeptide, which occurs sharply and reversibly at a specific temperature called the lower critical solution temperature (LCST) or the inverse transition temperature (Tₜ). Below the transition temperature (LCST or Tₜ), the thermally responsive polypeptides, (or polypeptides comprising a thermally responsive polypeptide) may be highly soluble. Upon heating above the transition temperature, thermally responsive polypeptides hydrophobically may collapse and aggregate, forming a separate, gel-like phase.

The thermally responsive polypeptides can phase transition at a variety of temperatures and concentrations. Thermally responsive polypeptides, for example, ELP, may not affect the binding or potency of the binding polypeptides. Thermally responsive polypeptides may allow the fusion protein to be tuned by a user to any number of desired transition temperatures, molecular weights, and formats.

Thermally responsive polypeptides may exhibit inverse phase transition behavior and thus, the fusion protein comprising the thermally responsive polypeptide may exhibit inverse phase transition behavior. Inverse phase transition behavior may be used to form drug depots within a tissue of a subject for controlled (slow) release of the fusion protein. Inverse phase transition behavior may also enable purification of the fusion protein using inverse transition cycling, thereby eliminating the need for chromatography.

### Binding Polypeptide

The binding polypeptide (or “targeting polypeptide”) may comprise any polypeptide that is capable of binding at least one target. The binding polypeptide may bind at least one target. “Target” may be an entity capable of being bound by the binding polypeptide. Targets may include, for example, another polypeptide, a cell surface receptor, a carbohydrate, an antibody, a small molecule, or a combination thereof. The target may be a biomarker. The target may be activated through agonism or blocked through antagonism. The binding polypeptide may specifically bind the target. By binding target, the binding polypeptide may act as a targeting moiety, an agonist, an antagonist, or a combination thereof. In some embodiments, the binding polypeptide domain binds TRAILR-2. “TRAIL receptor 2” or “TRAILR-2” refers to the TNF-Related Apoptosis-Inducing Ligand (TRAIL) Receptor 2 protein. Upon binding TRAIL or other agonists, TRAILR-2 activates apoptosis, or programmed cell death, in tumor cells. In some embodiments, the binding polypeptide domain binds epidermal growth factor receptor (EGFR). Upon binding epidermal growth factor (EGF) and other growth factor ligands, EGFR activates signal transduction pathways that promote cell proliferation.

The binding polypeptide may be a monomer that binds to a target. The monomer may bind one or more targets. The binding polypeptide may form an oligomer. The binding polypeptide may form an oligomer with the same or different binding polypeptides. The oligomer may bind to a target. The oligomer may bind one or more targets. One or more monomers within an oligomer may bind one or more targets. In some embodiments, the fusion protein is multivalent. In some embodiments, the fusion protein binds multiple targets. In some embodiments, the activity of the binding polypeptide alone is the same as the activity of the binding protein when part of a fusion protein.

In some embodiments, the binding polypeptide comprises one or more scaffold proteins. As used herein, “scaffold protein” refers to one or more polypeptide domains with relatively stable and defined three-dimensional structures. Scaffold proteins may further have the capacity for affinity engineering. In some embodiments, the scaffold protein has been engineered to bind a particular target. The scaffold proteins may be the same or different.

In some embodiments, the scaffold protein comprises a fibronectin domain. Fibronectin is a high-molecular weight glycoprotein of the extracellular matrix that binds to membrane-spanning receptor proteins called integrins. Fibronectin binds extracellular matrix components such as collagen, fibrin, and heparan sulfate proteoglycans. Human fibronectin exists as a protein dimer, comprising two nearly identical polypeptide chains linked by a pair of C-terminal disulfide bonds. Each human fibronectin subunit contains three domains: type I, II, and III. Fibronectin type III (Fn3) refers to the third of the three types of internal repeats in human fibronectin. This domain is often referred to as a scaffold protein because it contains three CDR-like (complementarity determining region) loops that can be engineered to bind a protein of interest using molecular biology techniques. In some embodiments, the fibronectin domain comprises Tn3. “Tn3” or “Tn3 scaffold” refers to an Fn3 domain from human tenascin C. Tn3 may comprise an amino acid sequence consisting of SEQ ID NO: 62. In some embodiments, Tn3 binds TRAIL receptor 2 (SEQ ID NO: 68). In one embodiment, the binding protein comprises a type III domain from human fibronectin (Fn3) (SEQ ID NO: 60). In another embodiment, the binding protein comprises Tn3 (SEQ ID NO: 62). In another embodiment, the binding protein comprises the alpha helical Z domain of Staphylococcus aureus protein A (SEQ ID NO: 64). In other embodiments, the binding polypeptide may comprise one or more proteins selected from, for example, anti-EGFR binding protein, DARPINS, knottins, or scFvs.

In some embodiments, the binding polypeptide comprises an amino acid sequence comprising Arg-Gly-Asp-Ser (RGDS). In another embodiment, the binding polypeptide comprises an amino acid sequence Gly-Arg-Gly-Asp-Ser-Pro-Ala-Ser (GRGDSPAS; SEQ ID NO: 76). In some embodiments, the binding polypeptide comprises a plurality of amino acid sequences consisting of SEQ ID NO: 60-64, 74-78. The amino acid sequence of SEQ ID NO: 60-64, 74-78 may be present anywhere within the binding polypeptide. In some embodiments, the amino acid sequence of SEQ ID NO: 60-64, 74-78 may be repeated in tandem within the binding polypeptide.

Other examples of binding proteins comprise one or more of a ErbB2 receptor binding protein (ANHP) with a sequence of SEQ ID NO:74; a cell-binding peptide (GRGDSPAS) with a sequence of SEQ ID NO:76; an adeno associated virus (AAV) binding protein (PKD2) with a sequence of SEQ ID NO:112; an adenovirus (AdV) binding protein (CAR) with a sequence of SEQ ID NO: 114; a lentivirus (LV) binding protein CR2 with a sequence of SEQ ID NO: 116; a lentivirus (LV) binding protein CR3 with a sequence of SEQ ID NO: 118; or an albumin binding protein (ABP) with a sequence of SEQ ID NO: 120.

### Linker

In some embodiments, the fusion protein further includes at least one linker. In some embodiments, the fusion protein includes more than one linker. In such embodiments, the linkers may be the same or different from one another. The fusion protein may include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, at least 90, at least 95, or at least 100 linkers. The fusion protein may include less than 500, less than 400, less than 300, or less than 200 linkers. The fusion protein may include between 1 and 1000, between 10 and 900, between 10 and 800, or between 5 and 500 linkers.

The linker may be positioned in between a binding polypeptide and an unstructured polypeptide, in between binding polypeptides, in between unstructured polypeptides, or a combination thereof. Multiple linkers may be positioned adjacent to one another. Multiple linkers may be positioned adjacent to one another and in between the binding polypeptide and the unstructured polypeptide.

The linker may be a polypeptide of any amino acid sequence and length. The linker may act as a spacer peptide. The linker may occur between polypeptide domains. The linker may sufficiently separate the binding domains of the binding polypeptide while preserving the activity of the binding domains. In some embodiments, the linker comprises charged amino acids. In some embodiments, the linker is flexible. In some embodiments, the linker comprises at least one glycine and at least one serine. In some embodiments, the linker comprises an amino acid sequence consisting of (Gly₄Ser)₃ (SEQ ID NO: 66). In some embodiments, the linker comprises at least one proline.

### Polynucleotides

Further provided are polynucleotides encoding the fusion proteins detailed herein. A vector may include the polynucleotide encoding the fusion proteins detailed herein. To obtain expression of a polypeptide, one typically subclones the polynucleotide encoding the polypeptide into an expression vector that contains a promoter to direct transcription, a transcription/translation terminator, and if for a nucleic acid encoding a protein, a ribosome binding site for translational initiation. An example of a vector is pET24 (SEQ ID NO: 121). Suitable bacterial promoters are well known in the art. Further provided is a host cell transformed or transfected with an expression vector comprising a polynucleotide encoding a fusion protein as detailed herein. Bacterial expression systems for expressing the protein are available in, e.g., E. coli, Bacillus sp., and Salmonella (Paiva et al., Gene 1983, 22, 229-235; Mosbach et al., Nature 1983, 302, 543-545). Kits for such expression systems are commercially available. Eukaryotic expression systems for mammalian cells, yeast, and insect cells are well known in the art and are commercially available. Retroviral expression systems can be used in the present invention. In some embodiments, the fusion protein comprises repeats or single sequences of one or more of SEQ ID NO: 18, 20, 22, 24, 26, 28, 30, 34, 36, 38, 40, 42, 44, 46, 48, 50, 60, 62, 64, 74, 76, or 78. In some embodiments, the fusion protein comprises repeats or single sequences of one or more of a polypeptide encoded by a polynucleotide sequence of any one of SEQ ID NO: 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, or 79. In some embodiments, the fusion protein comprises a polypeptide comprising an amino acid sequence of any one of SEQ ID NO: 80-110.

### Nanoparticles

Nanoparticles comprising one or more of the fusion proteins described herein can be produced by self-assembly of the fusion proteins. As described herein, the di-block identity and number of repeats influences nanoparticle formation.

### Crosslinkers

In one aspect, the nanoparticle is crosslinked to improve its stability and half-life in biological media. Crosslinking can be achieved by chemical methods targeting primary amine, carboxyl, sulfhydryl, or carbonyl moieties. Exemplary crosslinkers includes carbodiimide (e.g., EDC), NHS esters, imidoesters (pentafluorophenyl esters, hydroxymethyl phosphine), maleimides, haloacetyls (e.g., bromo- or iodo-), pyridyldisulfides, thiosulfonates, vinylsulfones, hydrazine, alkoxyamines, diazirines, aryl azides, isocyanates, formaldehyde, glutaraldehyde, among others. In other aspect, crosslinking can be accomplished by incorporating natural amino acids capable of cross-linking (cysteines to form cystines) or modified amino acids or chemically reactive amino acids that can be activated to form cross links.

A typical crosslinker used herein is p-azido-L-phenylalanine (pAzF). Crosslinking is accomplished by light activation of the N3 bond creating free radicals that can insert at any peptide bond or resolve in the presence of another radical N3 group. Solutions are prepared by resuspending the peptides from lyophilized powder to working concentrations, typically greater than 50 nM and exposed to high intensity UV-light for 0.1-30 sec. Chemical crosslinking can be used where the chemical linker is lyophilized with the diblock peptide or added after resuspension.

### Administration

The nanoparticles comprising one or more fusion proteins as detailed herein can be formulated in accordance with standard techniques well known to those skilled in the pharmaceutical art to form a therapeutic agent or targeted delivery agent. Such compositions comprising nanoparticles comprising one or more fusion proteins can be administered in dosages and by techniques well known to those skilled in the medical arts taking into consideration such factors as the age, sex, weight, and condition of the particular subject, and the route of administration.

The nanoparticles comprising one or more fusion proteins can be administered prophylactically or therapeutically. In prophylactic administration, the nanoparticle can be administered in an amount sufficient to induce a response. In therapeutic applications, the nanoparticles are administered to a subject in need thereof in an amount sufficient to elicit a therapeutic effect. An amount adequate to accomplish this is defined as “therapeutically effective dose.” Amounts effective for this use will depend on, e.g., the particular composition of the nanoparticle regimen administered, the manner of administration, the stage, and severity of the disease, the general state of health of the patient, and the judgment of the prescribing physician.

The nanoparticle can be administered by methods well known in the art as described in Donnelly et al. Ann. Rev. Immunol. 1997, 75, 617-648; Feigner et al., U.S. Pat. No. 5,580,859; Feigner, U.S. Pat. No. 5,703,055; and Carson et al., U.S. Pat. No. 5,679,647, the contents of each of which are incorporated herein by reference in their entirety. The nanoparticle can be complexed to particles or beads that can be administered to an individual, for example, using a vaccine gun. One skilled in the art would know that the choice of a pharmaceutically acceptable carrier, including a physiologically acceptable compound, depends, for example, on the route of administration.

The nanoparticles can be delivered via a variety of routes. Typical delivery routes include parenteral administration, e.g., intradermal, intramuscular, or subcutaneous delivery. Other routes include oral administration, intranasal, intravaginal, transdermal, intravenous, intraarterial, intratumoral, intraperitoneal, and epidermal routes. In some embodiments, the nanoparticle is administered intravenously, intraarterially, or intraperitoneally to the subject.

The nanoparticle can be a liquid preparation such as a suspension, syrup, or elixir. The nanoparticle can be incorporated into liposomes, microspheres, or other polymer matrices (such as by a method described in Feigner et al., U.S. Pat. No. 5,703,055; Gregoriadis, Liposome Technology, Vols. I to III (2^{nd} ed. 1993), the contents of which are incorporated herein by reference in their entirety). Liposomes can consist of phospholipids or other lipids, and can be nontoxic, physiologically acceptable, and metabolizable carriers that are relatively simple to make and administer.

The nanoparticle may be used as a vaccine. The vaccine can be administered via electroporation, such as by a method described in U.S. Pat. No. 7,664,545, which is incorporated herein by reference. The electroporation can be by a method or apparatus described in U.S. Pat. Nos. 6,302,874; 5,676,646; 6,241,701; 6,233,482; 6,216,034; 6,208,893; 6,192,270; 6,181,964; 6,150,148; 6,120,493; 6,096,020; 6,068,650; and 5,702,359, the contents of each of which are incorporated herein by reference in their entirety. The electroporation can be carried out via a minimally invasive device.

In some embodiments, the nanoparticle is administered in a controlled release formulation. In some embodiments, the nanoparticle comprises one or more thermally responsive polypeptides, the thermally responsive polypeptide having a transition temperature such that the nanoparticle remains soluble prior to administration and such that the nanoparticle transitions upon administration to a gel-like depot in the subject. In some embodiments, the nanoparticle comprises one or more fusion proteins comprising one or more thermally responsive polypeptides, the thermally responsive polypeptide having a transition temperature such that the fusion protein remains soluble at room temperature and such that the fusion protein transitions upon administration to a gel-like depot in the subject. For example, in some embodiments, the fusion protein comprises one or more thermally responsive polypeptides, the thermally responsive polypeptide having a transition temperature between room temperature (about 25° C.) and body temperature (about 37° C.), whereby the fusion protein can be administered to form a depot. As used herein, “depot” refers to a gel-like composition comprising a fusion protein that releases the fusion protein overtime. In some embodiments, the nanoparticle can be injected subcutaneously or intratumorally to form a depot (coacervate). The depot may provide controlled (slow) release of the nanoparticle. The depot may provide slow release of the nanoparticle into the circulation or the tumor, for example. In some embodiments, the nanoparticle may be released from the depot over a period of at least about 1 day, at least about 2 days, at least about 3 days, at least about 4 days, at least about 5 days, at least about 6 days, at least about 7 days, at least about 1 week, at least about 1.5 weeks, at least about 2 weeks, at least about 2.5 weeks, at least about 3.5 weeks, at least about 4 weeks, or at least about 1 month.

### Detection

As used herein, the term “detect” or “determine the presence of” refers to the qualitative measurement of undetectable, low, normal, or high concentrations of one or more nanoparticles, targets, or nanoparticles bound to target. Detection may include in vitro, ex vivo, or in vivo detection. Detection may include detecting the presence of one or more nanoparticles comprising one or more nanoparticles or targets versus the absence of the one or more nanoparticle or targets. Detection may also include quantification of the level of one or more nanoparticles or targets. The terms “quantify” or “quantification” may be used interchangeably, and may refer to a process of determining the quantity or abundance of a substance (e.g., nanoparticle or target), whether relative or absolute. Any suitable method of detection falls within the general scope of the present disclosure. In some embodiments, the nanoparticle comprises a reporter attached thereto for detection. In some embodiments, the nanoparticle is labeled with a reporter. In some embodiments, detection of a nanoparticle bound to a target may be determined by methods including but not limited to, band intensity on a Western blot, flow cytometry, radiolabel imaging, cell binding assays, activity assays, SPR, immunoassay, or by various other methods known in the art.

In some embodiments, including those wherein the nanoparticle is an antibody mimic for binding and/or detecting a target, any immunoassay may be utilized. The immunoassay may be an enzyme-linked immunoassay (ELISA), radioimmunoassay (RIA), a competitive inhibition assay, such as forward or reverse competitive inhibition assays, a fluorescence polarization assay, or a competitive binding assay, for example. The ELISA may be a sandwich ELISA. Specific immunological binding of the f nanoparticle to the target can be detected via direct labels, attached to the nanoparticle or via indirect labels, such as alkaline phosphatase or horseradish peroxidase. The use of immobilized f nanoparticle may be incorporated into the immunoassay. The nanoparticles may be immobilized onto a variety of supports, such as magnetic or chromatographic matrix particles, the surface of an assay plate (such as microtiter wells), pieces of a solid substrate material, and the like. An assay strip can be prepared by coating the nanoparticle or plurality of nanoparticles in an array on a solid support. This strip can then be dipped into the test biological sample and then processed quickly through washes and detection steps to generate a measurable signal, such as a colored spot.

### Methods of Treating a Disease

The present invention is directed to a method of treating a disease in a subject in need thereof. The method may comprise administering to the subject an effective amount of the nanoparticle comprising one or more nanoparticles as described herein. The disease may be selected from cancer, metabolic disease, autoimmune disease, cardiovascular disease, and orthopedic disorders. In some embodiments, the disease is a disease associated with a target of the at least one binding polypeptide.

Metabolic disease may occur when abnormal chemical reactions in the body alter the normal metabolic process. Metabolic diseases may include, for example, insulin resistance, non-alcoholic fatty liver diseases, type 2 diabetes, insulin resistance diseases, cardiovascular diseases, arteriosclerosis, lipid-related metabolic disorders, hyperglycemia, hyperinsulinemia, hyperlipidemia, and glucose metabolic disorders.

Autoimmune diseases arise from an abnormal immune response of the body against substances and tissues normally present in the body. Autoimmune diseases may include, but are not limited to, lupus, rheumatoid arthritis, multiple sclerosis, insulin dependent diabetes mellitis, myasthenia gravis, Grave's disease, autoimmune hemolytic anemia, autoimmune thrombocytopenia purpura, Goodpasture's syndrome, pemphigus vulgaris, acute rheumatic fever, post-streptococcal glomerulonephritis, polyarteritis nodosa, myocarditis, psoriasis, Celiac disease, Crohn's disease, ulcerative colitis, and fibromyalgia.

Cardiovascular disease is a class of diseases that involve the heart or blood vessels. Cardiovascular diseases may include, for example, coronary artery diseases (CAD) such as angina and myocardial infarction (heart attack), stroke, hypertensive heart disease, rheumatic heart disease, cardiomyopathy, heart arrhythmia, congenital heart disease, valvular heart disease, carditis, aortic aneurysms, peripheral artery disease, and venous thrombosis.

Orthopedic disorders or musculoskeletal disorders are injuries or pain in the body's joints, ligaments, muscles, nerves, tendons, and structures that support limbs, neck, and back. Orthopedic disorders may include degenerative diseases and inflammatory conditions that cause pain and impair normal activities. Orthopedic disorders may include, for example, carpal tunnel syndrome, epicondylitis, and tendinitis. Cancers may include, but are not limited to, breast cancer, colorectal cancer, colon cancer, lung cancer, prostate cancer, testicular cancer, brain cancer, skin cancer, rectal cancer, gastric cancer, esophageal cancer, sarcomas, tracheal cancer, head and neck cancer, pancreatic cancer, liver cancer, ovarian cancer, lymphoid cancer, cervical cancer, vulvar cancer, melanoma, mesothelioma, renal cancer, bladder cancer, thyroid cancer, bone cancers, carcinomas, sarcomas, and soft tissue cancers. In some embodiments, the cancer is colorectal cancer. In some embodiments, the cancer is colorectal adenocarcinoma.

One application of protein therapeutics is cancer treatment. In specific embodiments, the present invention provides a method for using scaffold proteins in developing antibody mimetics for oncological targets of interest. With the emergence of scaffold protein engineering come the possibilities for designing potent protein drugs that are unhindered by steric and architectural limitations. Although potent protein drugs can be invaluable for diagnostics or treatments, successful delivery to the target region can pose a great challenge.

### Methods of Diagnosing a Disease

Provided herein are methods of diagnosing a disease. The methods may include administering to the subject a nanoparticle comprising one or more fusion proteins as described herein and detecting binding of the nanoparticle to a target to determine presence of the target in the subject. The presence of the target may indicate the disease in the subject. In other embodiments, the methods may include contacting a sample from the subject with a nanoparticle as described herein, determining the level of a target in the sample, and comparing the level of the target in the sample to a control level of the target, wherein a level of the target different from the control level indicates disease in the subject. In some embodiments, the disease is selected from cancer, metabolic disease, autoimmune disease, cardiovascular disease, and orthopedic disorders, as detailed above. In some embodiments, the target comprises a disease marker or biomarker. In some embodiments, the nanoparticle may act as an antibody mimic for binding or detecting a target.

### Methods of Determining the Presences of a Target

Provided herein are methods of determining the presence of a target in a sample. The methods may include contacting the sample with a nanoparticle comprising one or more fusion proteins as described herein under conditions to allow a complex to form between the nanoparticle and the target in the sample and detecting the presence of the complex. Presence of the complex may be indicative of the target in the sample. In some embodiments, the nanoparticle is labeled with a reporter for detection.

In some embodiments, the sample is obtained from a subject and the method further includes diagnosing, prognosticating, or assessing the efficacy of a treatment of the subject.

When the method includes assessing the efficacy of a treatment of the subject, then the method may further include modifying the treatment of the subject as needed to improve efficacy.

### Methods of Determining the Effectiveness of a Treatment

Provided herein are methods of determining the effectiveness of a treatment for a disease in a subject in need thereof. The methods may include contacting a sample from the subject with a nanoparticle comprising a fusion protein as detailed herein under conditions to allow a complex to form between the nanoparticle and a target in the sample, determining the level of the complex in the sample, wherein the level of the complex is indicative of the level of the target in the sample, and comparing the level of the target in the sample to a control level of the target, wherein if the level of the target is different from the control level, then the treatment is determined to be effective or ineffective in treating the disease.

Time points may include prior to onset of disease, prior to administration of a therapy, various time points during administration of a therapy, and after a therapy has concluded, or a combination thereof. Upon administration of the nanoparticle comprising one or more fusion proteins to the subject, the nanoparticle may bind a target, wherein the presence of the target indicates the presence of the disease in the subject at the various time points. In some embodiments, the target comprises a disease marker or biomarker. In some embodiments, the nanoparticle may act as an antibody mimic for binding and/or detecting a target. Comparison of the binding of the nanoparticle to the target at various time points may indicate whether the disease has progressed, whether the diseased has advanced, whether a therapy is working to treat or prevent the disease, or a combination thereof.

In some embodiments, the control level corresponds to the level in the subject at a time point before or during the period when the subject has begun treatment, and the sample is taken from the subject at a later time point. In some embodiments, the sample is taken from the subject at a time point during the period when the subject is undergoing treatment, and the control level corresponds to a disease-free level or to the level at a time point before the period when the subject has begun treatment. In some embodiments, the method further includes modifying the treatment or administering a different treatment to the subject when the treatment is determined to be ineffective in treating the disease.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A composition comprising a protein nanoparticle comprising a fusion protein comprising at least one binding polypeptide and at least one unstructured polypeptide.
Clause 2. The composition of clause 1, wherein the fusion protein comprises a plurality of unstructured polypeptides.
Clause 3. The composition of clause 1 or 2, wherein the fusion protein comprises a plurality of targeting polypeptides.
Clause 4. The composition of any one of clauses 1-3, wherein the unstructured polypeptides comprise a di-block peptide.
Clause 5. The composition of any one of clauses 1-4, wherein the unstructured polypeptides comprise a di-block of a core polypeptide and a corona polypeptide.
Clause 6. The composition of any one of clauses 1-5, wherein the unstructured polypeptides comprise COREₙ-CORONAₘ, where n is 20-200 repeats and m is 40-200 repeats.
Clause 7. The composition of any one of clauses 1-6, wherein the core polypeptide comprises the sequence QYPSDGRG (SEQ ID NO: 1); GRGDQPYQ (SEQ ID NO: 2); GRGDSPYQ (SEQ ID NO: 3); GRGDSPYS (SEQ ID NO: 4); GRGDQPYS (SEQ ID NO: 5): GRGDSP[3Y:V]S (SEQ ID NO: 6): GRGDSP(Y:V]S (SEQ ID NO: 7); or combinations thereof.
Clause 8. The composition of any one of clauses 1-7, wherein the corona polypeptide comprises the sequence VPG[A:G]G (SEQ ID NO: 8); VPGSG (SEQ ID NO: 9); VPGVG (SEQ ID NO: 10); VPQQG (SEQ ID NO: 11); GRGDSPAS (SEQ ID NO: 12); GRGDSPIS (SEQ ID NO: 13): GRGDSPVS (SEQ ID NO: 14): GRGDQPHN (SEQ ID NO: 15); GRGDNPHQ (SEQ ID NO: 16); GRGDSPV (SEQ ID NO: 17); or combinations thereof.
Clause 9. The composition of any one of clauses 1-8, wherein the core polypeptide comprises the sequence (RLP)ₙ (SEQ ID NO: 1), where n is 20-200 repeats.
Clause 10. The composition of any one of clauses 1-9, wherein the corona polypeptide comprises the sequence (ELP)m (SEQ ID NO: 8), where m is 40-200 repeats.
Clause 11. The composition of any one of clauses 1-10, wherein the di-block comprises:

RLP40-ELP40 (SEQ ID NO: 83);

RLP40-ELP80 (SEQ ID NO: 84);

RLP40-ELP160 (SEQ ID NO: 82);

RLP60-ELP80 (SEQ ID NO: 85):

RLP80-ELP80 (SEQ ID NO: 87);

RLP80-ELP160 (SEQ ID NO: 86); or

RLP100-ELP80 (SEQ ID NO: 88).
Clause 12. The composition of any one of clauses 1-11, wherein the targeting polypeptide comprises 2 kDa to 100 kDa polypeptide.
Clause 13. The composition of any one of clauses 1-12, wherein the targeting polypeptide comprises a type III domain from human fibronectin (Fn3) (SEQ ID NO: 60); aFn3 domain from human tenascin C (Tn3) (SEQ ID NO: 62); or a Z-domain of staphylococcal protein A (SEQ ID NO: 64).
Clause 14. The composition of any one of clauses 1-13, wherein the targeting polypeptide comprises a type III domain from human fibronectin (Fn3) (SEQ ID NO: 60).
Clause 15. The composition of any one of clauses 1-14, wherein the targeting polypeptide comprises a Fn3 domain from human tenascin C (Tn3) (SEQ ID NO: 62).
Clause 16. The composition of any one of clauses 1-15, wherein the targeting polypeptide comprises a Z-domain of staphylococcal protein A with a sequence comprising (SEQ ID NO: 64).
Clause 17. The composition of any one of clauses 1-16, wherein the core polypeptide is crosslinked.
Clause 18. A protein nanoparticle comprising a fusion protein comprising at least one binding polypeptide and at least one unstructured polypeptide.
Clause 19. The protein nanoparticle of clause 18, wherein the fusion protein comprises a plurality of unstructured polypeptides.
Clause 20. The protein nanoparticle of clauses 18 or 19, wherein the fusion protein comprises a plurality of binding polypeptides.
Clause 21. The protein nanoparticle of any one of clauses 18-20, wherein the unstructured polypeptides comprise a di-block peptide.
Clause 22. The protein nanoparticle of any one of clauses 18-21, wherein the unstructured polypeptides comprise a di-block of a core polypeptide and a corona polypeptide.
Clause 23. The protein nanoparticle of any one of clauses 18-22, wherein the unstructured polypeptides comprise COREₙ-CORONAₘ, where n is 20-200 repeats and m is 40-200 repeats.
Clause 24. The protein nanoparticle of any one of clauses 18-23, wherein the core polypeptide comprises the sequence QYPSDGRG (SEQ ID NO: 1); GRGDQPYQ (SEQ ID NO: 2); GRGDSPYQ (SEQ ID NO: 3); GRGDSPYS (SEQ ID NO: 4); GRGDQPYS (SEQ ID NO: 5); GRGDSP[3Y:V]S (SEQ ID NO: 6); GRGDSP(Y:V]S (SEQ ID NO: 7); or combinations thereof.
Clause 25. The protein nanoparticle of any one of clauses 18-24, wherein the repeating core polypeptide sequence is interspersed with at least 1 but no more than 10 non-canonical amino acids selected from azidophenylalanine, acetylphenylalanine, propargyloxyphenylalanine, acetylphenylalanine, or azidohomoalanine.
Clause 26. The protein nanoparticle of any one of clauses 18-25, wherein the corona polypeptide comprises the sequence VPG[A:G]G (SEQ ID NO: 8); VPGSG (SEQ ID NO: 9); VPGVG (SEQ ID NO: 10); VPQQG (SEQ ID NO: 11); GRGDSPAS (SEQ ID NO: 12); GRGDSPIS (SEQ ID NO: 13); GRGDSPVS (SEQ ID NO: 14); GRGDQPHN (SEQ ID NO: 15); GRGDNPHQ (SEQ ID NO: 16); GRGDSPV (SEQ ID NO: 17); or combinations thereof.
Clause 27. The protein nanoparticle of any one of clauses 18-26, wherein the core polypeptide comprises the sequence (RLP)˜ (SEQ ID NO: 1), where n is 20-200 repeats.
Clause 28. The protein nanoparticle of any one of clauses 18-27, wherein the corona polypeptide comprises the sequence (ELP)m (SEQ ID NO: 8), where m is 40-200 repeats.
Clause 29. The protein nanoparticle of any one of clauses 18-28, wherein the di-block comprises:

RLP40-ELP40 (SEQ ID NO: 83);

RLP40-ELP80 (SEQ ID NO: 84);

RLP40-ELP160 (SEQ ID NO: 82);

RLP60-ELP80 (SEQ ID NO: 85);

RLP80-ELP80 (SEQ ID NO: 87);

RLP80-ELP160 (SEQ ID NO: 86); or

RLP100-ELP80 (SEQ ID NO: 88).
Clause 30. The protein nanoparticle of any one of clauses 18-29, wherein the targeting polypeptide comprises 2 kDa to 100 kDa polypeptide.
Clause 31. The protein nanoparticle of any one of clauses 18-30, wherein the binding polypeptide comprises a type III domain from human fibronectin (Fn3) (SEQ ID NO: 60); aFn3 domain from human tenascin C (Tn3) (SEQ ID NO: 62); or a Z-domain of staphylococcal protein A (SEQ ID NO: 64).
Clause 32. The protein nanoparticle of any one of clauses 18-30, wherein the binding polypeptide comprises a comprises a type III domain from human fibronectin (Fn3) (SEQ ID NO: 60).
Clause 33. The protein nanoparticle of any one of clauses 18-30, wherein the binding polypeptide comprises a Fn3 domain from human tenascin C (Tn3) (SEQ ID NO: 62).
Clause 34. The protein nanoparticle of any one of clauses 18-30, wherein the binding polypeptide comprises a Z-domain of staphylococcal protein A with a sequence comprising (SEQ ID NO: 64).
Clause 35. The protein nanoparticle of any one of clauses 18-30, wherein the binding polypeptide comprises an ErbB2 receptor binding protein (ANHP) (SEQ ID NO: 74).
Clause 36. The protein nanoparticle of any one of clauses 18-30, wherein the binding polypeptide comprises a cell-binding peptide (GRGDSPAS) (SEQ ID NO: 76).
Clause 37. The protein nanoparticle of any one of clauses 18-30, wherein the binding polypeptide comprises an adeno associated virus (AAV) binding protein (PKD2) (SEQ ID NO: 112).
Clause 38. The protein nanoparticle of any one of clauses 18-30, wherein the binding polypeptide comprises an adenovirus (AdV) binding protein (CAR) (SEQ ID NO: 114).
Clause 39. The protein nanoparticle of any one of clauses 18-30, wherein the binding polypeptide comprises a lentivirus (LV) binding protein (CR2) (SEQ ID NO: 116) or (CR3) (SEQ ID NO: 118).
Clause 40. The protein nanoparticle of any one of clauses 18-30, wherein the binding polypeptide comprises an albumin binding protein (ABP) (SEQ ID NO: 120).
Clause 41. The protein nanoparticle of any one of clauses 22-40 where the core is covalently crosslinked using light or other click-chemistry compatible linkers.
Clause 42. The protein nanoparticle of any one of clauses 22-41, wherein the core polypeptide is crosslinked.
Clause 43. The protein nanoparticle of any one of clauses 18-42, wherein the nanoparticle encapsulates one or more small molecule drugs within its interior.
Clause 44. The protein nanoparticle of any one of clauses 18-43, wherein the fusion protein further comprises a therapeutic protein.
Clause 45. The protein nanoparticle of any one of clauses 18-44, wherein the composition is a therapeutic agent, targeted-delivery agent, separation agent, or purification agent.
Clause 46. A therapeutic agent comprising the protein nanoparticle of any one of clauses 18-45.
Clause 47. A method of targeting a therapeutic to a cell comprising administering the protein nanoparticle of any one of clauses 18-45.
Clause 48. A method of delivering a therapeutic to a cell comprising administering the protein nanoparticle of any one of clauses 18-45.
Clause 49. A means for targeting a therapeutic to a cell comprising administering the protein nanoparticle of any one of clauses 18-45.
Clause 50. A means for delivering a therapeutic to a cell comprising administering the protein nanoparticle of any one of clauses 18-45.
Clause 51. A method for identifying a biomolecule comprising administering the protein nanoparticle of any one of clauses 18-45 that binds to the biomolecule.
Clause 52. A method of purifying a biomolecule comprising using the protein nanoparticle of any one of clauses 18-45 that binds to the biomolecule to isolate the biomolecule from a medium.
Clause 53. The method of clause 52, further comprising a triggered phase separation of the binding polypeptide to isolate the biomolecule from contaminants, wherein the trigger is selected from a modulation of temperature, salinity, light, pH, pressure, concentration of the binding polypeptide, concentration of the biomolecule, application of electromagnetic or acoustic waves, or addition of one or more excipients comprising one or more of cofactors, surfactants, crowding reagents, reducing agents, oxidizing agents, denaturing agents, or enzymes.
Clause 54. The method of clause 52, further comprising using centrifugation to separate dense phase separated proteins bound to the biomolecule from contaminant biomolecules.
Clause 55. The method of clause 52 or 54, further comprising using centrifugation to separate phase separated proteins bound to the biomolecule from contaminant biomolecules.
Clause 56. The method of any one of clauses 52-55, further comprising using the size of the phase separated droplets to isolate the biomolecule from contaminant species, wherein the size of the binding polypeptide bound to the biomolecule is at least 20 nm in diameter and no larger than 100 μm in diameter.
Clause 57. The method of any one of clauses 52-56, wherein the method comprising using flow filtration, membrane chromatography, analytical ultracentrifugation, high performance liquid chromatography, membrane chromatography, normal flow filtration, acoustic wave separation, centrifugation, counterflow centrifugation, and fast protein liquid chromatography to isolation the biomolecule-binding polypeptide complex from contaminant species on the basis of size.
Clause 58. A biomolecule comprising of at least one of a lipid, a cell, a protein, a nucleic acid, a carbohydrate or a viral particle, wherein the nucleic acid is a single stranded or double stranded DNA or RNA; the viral particle is selected from an adenovirus particle, an adeno-associated virus particle, a lentivirus particle, a retrovirus particle, a poxvirus particle, a measle virus particle, or herpesvirus particle; and the protein is selected from human albumin, monoclonal IgG antibodies, or Fc fusion antibodies.

### EXAMPLES

### Example 1

### Gene Synthesis

Plasmid genes were available from previous studies for RLP20 (SEQ ID NO: 18-19), RLP20-ELP80 (SEQ ID NO: 81), RLP40-ELP80 (SEQ ID NO: 84), RLP80-ELP80 (SEQ ID NO: 87), RLP100-ELP80 (SEQ ID NO: 88), and an Fn3 domain (SEQ ID NO: 60) that binds the αvβ3 integrin. This gene was then subsequently fused with the gene that encodes the Fn3 domain. Similarly, genes encoding RLP20-ELP80 (SEQ ID NO: 81), RLP40-ELP80 (SEQ ID NO: 84), RLP80-ELP80 (SEQ ID NO: 87) were cloned to the N-terminus of an Fn3 (SEQ ID NO: 60) with the same directional ligation method. After successful confirmation of gene assembly by Sanger fluorescent DNA sequencing, the plasmids harboring each construct were isolated and transformed into BL21(DE3) expression strain of E. coli. Aliquots of the cell stocks were stored at −80° C. until further use.

### Protein Purification

Each block polypeptide was expressed in BL21(DE3) E. coli using a previously published hyperexpression protocol. 5 mL bacterial cultures were grown overnight from frozen glycerol stocks and used to inoculate 1 L flasks of TB Dry, supplemented with 45 μg/mL kanamycin. The flasks were then incubated at 37° C. for 24 hours and 190 rpm. Each construct was purified using inverse transition cycling (ITC). Briefly, the cell suspension was centrifuged at 3,000 rpm for 10 min at 4° C., the cell pellet then resuspended in PBS and then lysed by sonication on ice for 2 min (10 s on, 40 s off) (Misonix S-4000; Farmingdale, N.Y.). Polyethyleneimine (PEI) 0.7% w/v was added to the lysate to precipitate nucleic acid contaminants. The supernatant was then subjected to multiple rounds of ITC as follows: the solution was kept on ice, and 3 M NaCl was added to isothermally trigger the phase transition of the RLP-ELP block co-polypeptide. The coacervate was then centrifuged for 20 min at 14,000×g at 30° C., the supernatant was decanted and discarded, and the pellet was resuspended in phosphate buffer. The dissolved product was cooled to 4° C., and then centrifuged for 10 min at 15,000×g at 4° C. to remove any insoluble contaminants. To remove excess salt from purified protein solutions, the samples were dialyzed against ddH₂O at 4° C. for at least 24 h using Spectrum™ Labs Spectra/Por™ 2 12-14 Standard RC Dry Dialysis Kits (Fisher Scientific, Waltham, Mass.). The proteins were then lyophilized and stored at −20° C. Purity of the block polypeptides was assessed by SDS-PAGE gel with SimplyBlue staining.

### Characterization of Phase Separation

### Temperature Dependent UV-Vis Spectrophotometry

Turbidity profiles were obtained for each of the constructs by recording the optical density as a function of temperature (1° C. min⁻¹ ramp) on a temperature-controlled UV-vis spectrophotometer (Cary 300 Bio; Varian Instruments; Palo Alto, Calif.). The transition temperature (Tₜ) was defined as the inflection point of the turbidity profile. Samples were measured in PBS at 10 μM. Because some of the block co-polypeptides which form larger micelles are slightly turbid when soluble, all measurements were taken after zeroing with PBS.

### Static and Dynamic Light Scattering

Static and dynamic light scattering measurements (SLS/DLS) were performed using an ALV/CGS-3 goniometer system (Langen, Germany). Samples for the ALV/CGS-3 goniometer system were prepared at a concentration of 10 μM in PBS and filtered through 0.45 μm Millex-GV filters into a 10 mm disposable borosilicate glass tube (Fischer). Simultaneous SLS and DLS measurements were obtained at 15° C. of the ELP for angles between 30°-150° at 5° increments, with each angle consisting of 3 runs for 15 s. SLS experiments were only conducted for self-assembling block co-polypeptides, since the molecular weight of a single block co-polypeptide chain is already known, and the R_{g} of a single chain is likely near or below the detection limit of the SLS instrument. The differential refractive index (dₙ/d_{c}) was determined by measuring the refractive index at different concentrations using an Abbemat 500 refractometer (Anton Paar, Graz, Austria). DLS data were analyzed by fitting the autocorrelation function with a cumulant fit, using the built-in ALV software. Hydrodynamic radius (Rₕ) was plotted against angle and extrapolated to zero. SLS data were analyzed by partial Zimm plots using ALVSTAT software in order to determine the R_{g} and molecular weight (MW).

### Temperature-Programmed Dynamic Light Scattering

Temperature-programmed dynamic light scattering experiments were carried out using a Dynapro plate reader (Wyatt Technology; Santa Barbara, Calif.) with samples filtered through 0.45 μm Millex-GV filters. Data were collected at increments of 1° C., and the cumulant fit hydrodynamic radius was taken as the radius. The Tₜ was defined as the temperature at which aggregates of size hundreds of nanometers were formed.

### Cryogenic Transmission Electron Microscopy

Cryo-TEM experiments were performed at Duke University's Shared Materials Instrumentation Facility (Durham, N.C.). Lacey holey carbon grids (Ted Pella, Redding, Calif.) were glow discharged in a PELCO EasiGlow Cleaning System (Ted Pella, Redding, Calif.). A 3 μL drop (10 μM RLPn-ELP80) was deposited onto the grid, blotted for 3 s with an offset of −3 mm, and vitrified in liquid ethane using the Vitrobot Mark III (FEI, Eindhoven, Netherlands). Prior to vitrification, the sample chamber was maintained at 15° C. and 100% relative humidity to prevent sample evaporation. Grids were transferred to a Gatan 626 cryoholder (Gatan, Pleasanton, Calif.) and imaged with a FEI Tecnai G2 Twin TEM (FEI, Eindhoven, Netherlands), operating at 80 keV. Feature sizes and spacing distances were measured in ImageJ by manual measurement of at least 25 particles.

### Surface Plasmon Resonance Spectrophotometry

The surface plasmon resonance experiments were performed using Biacore T200. Purified human αvβ3 integrin (Chemicon, Temecula, Calif.) were immobilized on research grade CM5 sensor chips using an amine coupling kit (BIAcore, Piscataway, N.J.). The integrin was diluted in 10 mM sodium acetate buffer (pH 4.5) for conjugation with a surface density of approximately 600 resonance units (RU). The measurements of binding events were performed using block co-polypeptide concentrations ranging between 2.5 and 10 μM. The block polypeptides were diluted in HBS-P buffer (10 mM HEPES, 140 mM NaCl, 0.005% Triton-X, pH 7.4) supplemented with 2 mM CaCl₂, and injected into the flow cells at a flow rate of 30 μL·min⁻¹ for 4 min. The complex was allowed for dissociation for 10 min. The surface was regenerated with 10 mM Glycine-HCl (pH 2.5) at a flow rate of 30 μL·min⁻¹ for 45 s, followed by 10 mM Glycine-HCl (pH 2.0) at a flow rate of 30 μL·min⁻¹ for 30 s. The surface was regenerated using 10 mM glycine-HCL (pH 2.0). Kinetic modeling and simulations were performed using BIAevaluation software with the heterogeneous ligand model for self-assembled proteins and with a 1:1 ligand model for the unimeric protein (RLP20-ELP80-Fn3). The equilibrium binding constants (K_{D1} and K_{D2}) for each experiment were calculated by dividing kinetic dissociation rate (k_{off}) by association rate (kₒₙ), from which the mean K_{D1/2} was derived. All SPR measurements were carried out at 25° C. The SPR measurements were carried out using polypeptide concentrations ranging between 2.5 and 10 μM. Goodness-of-fit was evaluated by analyzing residual plots and residual sum of squares.

### Flow Cytometry

Approximately 1×10⁸ cells were harvested from either K562 or K562+αvβ3 cell lines and resuspended into 1 mL of serum-free medium containing 10 μM of the various Fn3-decorated and control block polypeptides. LM609 antibody was also resuspended at 10 μM in serum-free medium. Micelles were prepared from a mixture of ˜10% Alexa 488 dye-labeled RLP-ELP block co-polypeptides and 90% unlabeled polypeptides on a molar basis. The cells were incubated at 37° C. with the labeled micelles for a specified time, then rinsed with 1 mL of Hanks Buffered Saline Solution (HBSS), collected by centrifugation at 500 RCF for 5 min at 20° C., and resuspended in HBSS+1% BSA. Cells were maintained on ice until they were analyzed by flow cytometry (BD Accuri C5). The cell fluorescence intensity of Alexa 488 (Green) was quantified after gating to remove cellular debris on unstained control samples.

### Confocal Microscopy

Approximately 1×10⁶ cells were harvested from either K562 or K562+αvβ3 cell lines and resuspended into 1 mL of serum-free medium containing 10 μM of the various decorated and undecorated block polypeptides. Cells were incubated at 37° C. for various times (20-240 min). After washing with HBSS thrice, 20 μL of cell suspension was added to a 384 well plate with a #1.5 coverslip on the bottom. Cells were imaged on a Zeiss 710 inverted confocal (Oberkochen, Germany) equipped with a live cell chamber maintained at 37° C. using a 40× oil immersion objective.

### Confocal Image Analysis

For analysis of percentage of cells that show uptake of the polypeptides, the fluorescent channel and DIC channel were isolated and analyzed independently. In the fluorescent channel, the lowest 10% of cell fluorescence was removed, to eliminate any autofluorescence from naïve K562 cells. Using this cutoff, locations and area of green fluorescence were identified using the fluorescent channel only. Total number of cells were then counted using the DIC channel.

### Fluorophore Labeling

The N-terminus and lysine residues in the RLP-ELP-Fn3 fusions were labeled with the NHS-ester derivative of Alexa488. To bias the reaction towards N-terminal labeling, the pH of the reaction mixture was adjusted to 8.3. The RLP-ELP block co-polypeptides, dissolved in 0.1 M sodium bicarbonate buffer (pH 8.3), were incubated with a molar excess of dye (with a dye-to-protein molar ratio depending on the total number of reactive groups in the proteins, which includes lysine residues and the N-terminus (e.g., dye:RLP₂₀=2:1, dye:RLP₂₀-Fn3 is 5:1), for 2 hours at room temperature with continuous agitation. Excess dye was removed with 3 rounds of dialysis over 3 days at 4° C. with a 1:500 volume ratio of reaction mixture to milli-Q water. The samples were lyophilized and stored at −20° C.

### Example 2

Block Co-Polypeptides with UCST and LCST Phase Behavior can be Combined to Create Micelles with Predictable Nanoscale Assembly.

The first area of investigation was the effect of hydrophilic weight fraction of an RLP-ELP block co-polypeptide. The core block sequence was (Gln-Tyr-Pro-Ser-Asp-Gly-Arg-Gly)-XX (RLPXX) (SEQ ID NO: 1) and the corona sequence was (Val-Pro-Gly-[Ala/Gly]-Gly)-YY (ELPYY) (SEQ ID NO: 8) where the guest ratio was a 50/50 split between Ala and Gly. The core block size was controlled to be 20, 40, 60 or 80 repeat units of (Gln-Tyr-Pro-Ser-Asp-Gly-Arg-Gly) and the corona block was 80 repeats of (Val-Pro-Gly-[Ala/Gly]-Gly)-YY (SEQ ID NO: 81, 84, 93, 87).

We understand some detail about the assembly of these polypeptides from our scattering experiments (Table 1). First, RLP20-ELP80 has a hydrodynamic radius of 5.5 nm, in accordance with a fully soluble ˜47 kDa polymer chain, and thus does not self-assemble. Second, RLP40-ELP80 and RLP60-ELP80 both self-assemble into structures with Rₕ less than 50 nm, R_{g} less than 40 nm, shape factors below 1 and aggregation numbers under 250. The radii, combined with the shape factors and the aggregation numbers, indicate that both RLP40-ELP80 and RLP60-ELP80 likely self-assemble into spherical micelles. Third, RLP80-ELP80 self-assembles into much larger structures, with hydrodynamic radii over 100 nm, radii of gyration above 140 nm, a shape factor around 1.2, and aggregation numbers in the thousands of chains. These results indicate that RLP80-ELP80 self-assembles into much larger, non-spherical structures.

TABLE 1
Light scattering data of RLPXX-ELP80 co-polypeptides

| | Hydrophilic | | | | |
|---|---|---|---|---|---|
| Block co-polypeptide | Content | N_{agg} | R_{g}(nm) | Rₕ (nm) | ρ |
| RLP20-ELP80 (SEQ | 64.7% | — | — | 5.5 | — |
| ID NO: 81) | | | | | |
| RLP40-ELP80 (SEQ | 47.4% | 68 | 29.9 | 33.3 | 0.89 |
| ID NO: 84) | | | | | |
| RLP60-ELP80 (SEQ | 37.5% | 231 | 23.8 | 36.7 | 0.65 |
| ID NO: 93) | | | | | |
| RLP80-ELP80 (SEQ | 30.9% | 2240 | 145.4 | 114.3 | 1.27 |
| ID NO: 87) | | | | | |

Cryo-TEM results confirm the suspicions of both dynamic and static light scattering (FIG. 4). Some important observations for all constructs are that the nanostructures are spatially very close to one another indicating that these structures are near the overlap regime. Sample preparation was at 10 μM, theoretically in the dilute regime. Therefore, the concentration of these structures is artificially increased by the vitrification process.

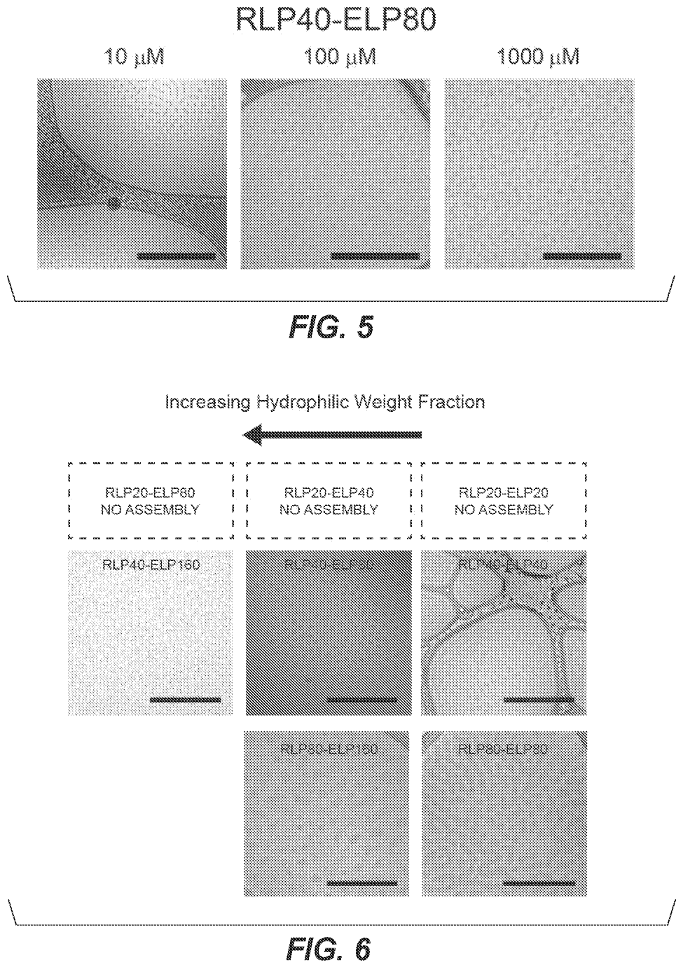

However, increasing the preparation concentration of RLP40-ELP80 to 100 μM and 1 mM did not seem to affect the observed structure by cryo-TEM (FIG. 5). Therefore, we expect that the self-assembled morphology has a wide range of concentration independence and that the observed shape in cryo-TEM is consistent with light scattering data. Finally, we were unable to visualize any of the corona chains for any of the RLP-ELPs sampled, likely due to their high water content and corresponding low contrast with water.

RLP40-ELP80 (SEQ ID NO: 84) and RLP60-ELP80 (SEQ ID NO: 93) (FIG. 4 both self-assemble into spherical micelles. This result was suggested by the DLS data and SLS and was confirmed by cryo-TEM. Measurements of core radii indicate that RLP40-ELP80 (SEQ ID NO: 84) and RLP60-ELP80 (SEQ ID NO: 93) cores are approximately 12.8 nm and 17.5 nm, consistent with a larger core-forming block leading to a larger micelle core. This is consistent with the larger values reported by light scattering, because Rₕ and R_{g} incorporate both the core and corona portions of the micelle, whereas only the micelle core is directly observed by cryo-TEM. The spacing was similar for both: 29.5 nm, which is consistent because both block co-polypeptides have the same morphology and same corona-forming ELP block.

Cryo-TEM reveals that RLP80-ELP80 (SEQ ID NO: 87) (FIG. 4) forms a different nanostructure. These block co-polypeptides form long, cylindrical structures. Again, an apparent increase in the core block size from 17.5 nm to 19.9 nm and an increase in the spacing of 28.9 nm to 34.7 nm is consistent with increasing size of the core block (although the core size increase is non-significant). These overlapping structures are more consistent with lamellae formation and therefore the aspect ratio of the cylinder is not observable with cryo-TEM at this concentration.

TABLE 2
Micelle core and inter-core spacing of RLPXX-ELP80
block co-polypeptides (mean ± standard deviation)

| Block polypeptide | Core radius (nm)^{-[a]} | Spacing (nm) |
|---|---|---|
| RLP40-ELP80 (SEQ | 12.8 ± 1.8 | 29.5 ± 4.8 |
| ID NO: 84) | | |
| RLP60-ELP80 (SEQ | 17.5 ± 2.7 | 28.9 ± 5.1 |
| ID NO: 93) | | |
| RLP80-ELP80 (SEQ | 19.9 ± 2.8 | 34.7 ± 4.2 |
| ID NO: 87) | | |

In order to test if overall hydrophilic weight fraction or block length was the main driving force of micelle morphology, overall length was changed while maintaining a specific hydrophilic weight fraction. This resulted in a comparison between overall hydrophilic weight fractions of 64.7% (RLP20-ELP80 (SEQ ID NO: 81) and RLP40-ELP-160 (SEQ ID NO: 82)), 47.4% (RLP20-ELP-40 (SEQ ID NO: 80), RLP40-ELP80 (SEQ ID NO: 84), RLP80-ELP-160 (SEQ ID NO: 86)) and 30.9% (RLP40-ELP-40 (SEQ ID NO: 83), RLP80-ELP80 (SEQ ID NO: 87)).

RLP20-ELP80 with a hydrophilic weight of 64.7% did not assemble as mentioned previously. However, doubling the block length of the corona and the core (RLP40-ELP160) (SEQ ID NO: 82) resulted in an assembled structure with a R_{g} of 70.8 nm, Rₕ of 92.3 nm and form factor of 0.8 indicating a spherical micelle morphology. This result was confirmed with cryo-TEM, which revealed spheres that had an average core radius of 11.0 nm and spacing of 22.3 nm between particle cores.

TABLE 3
Light scattering data of RLPXX-ELPYY co-polypeptides

| | Hydrophilic | | | | |
|---|---|---|---|---|---|
| Block co-polypeptide | Content | N_{agg} | R_{g}(nm) | Rₕ (nm) | ρ |
| RLP40-ELP160 (SEQ | 64.7% | 246 | 70.8 | 92.3 | 0.8 |
| ID NO: 82) | | | | | |
| RLP20-ELP40 (SEQ | 47.4% | — | — | 5.3 | — |
| ID NO: 80) | | | | | |
| RLP80-ELP160 (SEQ | 47.4% | 1728 | 78.2 | 93.5 | 0.84 |
| ID NO: 86) | | | | | |
| RLP40-ELP40 (SEQ | 30.9% | 91 | 56.2 | 52.6 | 1.13 |
| ID NO: 83) | | | | | |

RLP20-ELP40 (SEQ ID NO: 80), RLP80-ELP160 (SEQ ID NO: 86), are both expected to assemble into spherical micelles due to the overall hydrophilic weight fraction of 47.4% which showed spherical micelles with RLP40-ELP80 (SEQ ID NO: 84). RLP20-ELP40 however did not assemble and had a soluble Rₕ of 5.3 nm, consistent with a 32 kDa chain. This can be explained as a 32 kDa chain not having sufficient assembly domain size. RLP80-ELP160 (SEQ ID NO: 86) did assemble with R_{g} of 78.2 nm, Rₕ of 93.3 nm and form factor of 0.8 indicating spherical micelles.

Cryo-TEM imaging confirmed this result and provided interesting nanostructure information (FIG. 6). The core of these micelles had a radius of 29.3 nm with an intra-core spacing of 59.0 nm. This core dimension is much larger than RLP80-ELP80 (SEQ ID NO: 87) which adopted a cylindrical micelle formation indicating that the core of the larger spheres is more expanded in a spherical micelle than in a cylindrical micelle. This result can be directly predicted from the theory of synthetic polymer micelles as the chains of a sphere are expected to be more extended than in a rod-like conformation. Additionally, we observe a much larger intra-core spacing due to the doubling in the size of the ELP chain.

TABLE 4
Micelle core and inter-core spacing of RLPXX-ELPYY
block co-polypeptides (mean ± standard deviation)

| Block polypeptide | Core radius (nm)^{-[a]} | Spacing (nm) |
|---|---|---|
| RLP40-ELP160 (SEQ | 11.0 ± 1.6 | 22.3 ± 5.2 |
| ID NO: 82) | | |
| RLP80-ELP160 (SEQ | 29.3 ± 4.5 | 59.0 ± 13.2 |
| ID NO: 86) | | |
| RLP40-ELP40 (SEQ | 11.8 ± 1.6 | 19.5 ± 4.3 |
| ID NO: 83) | | |

Finally, RLP40-ELP40 (SEQ ID NO: 83) likely adopts a non-spherical geometry with a R_{g} of 56.2 nm, Rₕ of 52.6 nm and form factor greater than 1. This is comparable to RLP80-ELP80 which adopted a similar conformation. Both the core radius (11.8 nm) and the spacing (19.5 nm) are smaller than the larger polymer, which is consistent with the smaller core and corona chains. The core size is about the same size as the core radii of other assembled structures with different morphologies.

Another key design parameter besides the effect of hydrophilic weight fraction is the effect of corona hydrophilicity. Therefore, a more hydrophobic guest residue of Val (V) and more hydrophilic guest residue of Ser (S) were substituted into RLP40-ELP80, RLP60-ELP80, and RLP80-ELP80 (SEQ ID NO: 89-94). Our hypothesis is that the introduction of Val will reduce corona chain repulsion, leading from a more spherical to more rod-like assembly. A Ser would provide more chain repulsion and drive a rod-like to spherical transition.

The substitution of Ala/Gly for Val resulting in a spherical to worm shift in the light scattering data (Table 5). Comparing RLP40-ELP80 (SEQ ID NO: 84) (spherical) and RLP40-ELPV80 (SEQ ID NO: 92), RLP40-ELPV80 (SEQ ID NO: 92) has a higher aggregation number and a larger R_{g} value, resulting in a ρ>1. Since these two polymers have nearly identical molecular weight and the exact same chain length, we can surmise that RLP40-ELPV80 (SEQ ID NO: 92) is forming more elongated structures. A similar increase is seen with RLP40-ELPV80 compared to RLP40-ELP-80 (SEQ ID NO: 84). Just as with the Ala/Gly constructs, N_{agg}, R_{g}, and Rₕ increase as the core block length increases and yet the form factor remains >1, indicating that all the Val constructs are worm-like micelles.

TABLE 5
Light scattering data of RLPXX-ELPS80
and RLPXX-ELPV80 co-polypeptides

| | Hydrophilic | | | | |
|---|---|---|---|---|---|
| Block co-polypeptide | Content | N_{agg} | R_{g}(nm) | Rₕ (nm) | ρ |
| RLP40-ELPS80 (SEQ | 48.2% | 80 | 27.2 | 33.0 | 0.82 |
| ID NO: 89) | | | | | |
| RLP60-ELPS80 (SEQ | 38.3% | 244 | 27.5 | 43.5 | 0.6 |
| ID NO: 90) | | | | | |
| RLP80-ELPS80 (SEQ | 32.1% | 392 | 36.0 | 49.4 | 0.73 |
| ID NO: 91) | | | | | |
| RLP40-ELPV80 (SEQ | 48.9% | 213 | 33.1 | 31.7 | 1.04 |
| ID NO: 92) | | | | | |
| RLP60-ELPV80 (SEQ | 39.0% | 565 | 41.3 | 39.4 | 1.0 |
| ID NO: 93) | | | | | |
| RLP80-ELPV80 (SEQ | 32.8% | 693 | 94.4 | 71.0 | 1.31 |
| ID NO: 94) | | | | | |

On the other hand, RLP40-ELPS80 (SEQ ID NO: 89) has approximately the same N_{agg}, R_{g}, and Rₕ as RLP40-ELP80 (SEQ ID NO: 84). This would indicate that both constructs are spherical micelles. Interestingly, it appears the substitution of serine for alanine and glycine in RLP80-ELP80 has reduced the N_{agg}, R_{g}, Rₕ, so that the form factor is now <1. This would indicate that this substitution has led to a shift in morphology—from a wormlike micelle to a sphere. Just as with the Ala/Gly and Val constructs, N_{agg}, R_{q}, and Rₕ increase as the core block length increases and yet the form factor remains <1, indicating that all the serine constructs are spherical.

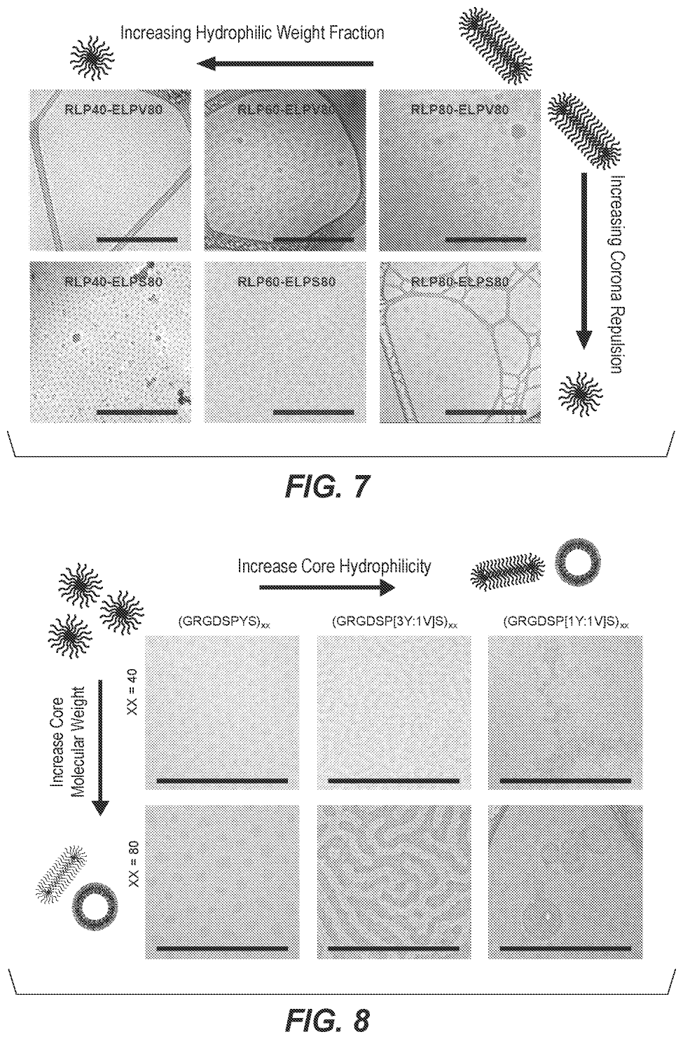

As suspected by the light scattering data, an Ala/Gly to Val substitution resulted in spherical to worm-like micelle shift according to cryo-TEM imaging. In FIG. 7 you can see that increasing the hydrophilic weight fraction, the worms get progressively longer, but the core radii remain approximately the same size (Table 6) and eventually become an interconnected network. The Ala/Gly substitution with Ser appears to have increased the corona repulsion so much that it can only form spherical micelles.

TABLE 6
Micelle core and inter-core spacing of RLPXX-ELPSYY and RLPXX-
ELPVYY block co-polypeptides (mean ± standard deviation)

| Block polypeptide | Core radius (nm)^{-[a]} | Spacing (nm) |
|---|---|---|
| RLP40-ELPS80 (SEQ | 13.1 ± 2.7 | 20.0 ± 2.6 |
| ID NO: 89) | | |
| RLP80-ELPS80 (SEQ | 14.6 ± 2.2 | 27.9 ± 4.0 |
| ID NO: 91) | | |
| RLP40-ELPV80 (SEQ | 14.5 ± 3.6 | 19.4 ± 4.4 |
| ID NO: 92) | | |
| RLP80-ELPV80 (SEQ | 14.2 ± 2.4 | 29.6 ± 3.8 |
| ID NO: 94) | | |

In addition to changes to the corona sequence to increase or decrease repulsion between chains (or perhaps changing the occupied volume of the chain), we sought to perform the same experiment, this time with the core sequence. As we know from previous studies, substituting Val for Tyr increases the saturation concentration and decreases the density of the dense phase. Thus, we hypothesized that this same substitution would create a similar effect in the core of a particle, increasing or decreasing the core occupied volume and perhaps changing the self-assembled structure. We made mutations to the RLP40-ELP80 (SEQ ID NO: 84) and RLP80-ELP80 (SEQ ID NO: 91) core repeat sequence, replacing (QYPSDGRG) (SEQ ID NO: 1) with (GRGDSP[Y]S) (SEQ ID NO: 6-7) where the Tyr in the new repeating unit is systematically replaced with Val. What we observed for both core molecular weights that a systematic replacement of Tyr along the backbone results in a transition from more spherical particles to more elongated scattering structures (FIG. 8). Specifically, with a core repeat number of 80, we observe all three transition states, spherical micelles that transition to worm like micelles or lamellae and vesicular structures. For this vesicular structure in particular, it appears to be in two phase equilibrium with worm like structures as there are additional areas of contrast throughout the various cryo-TEM imaging frames. The assembly of these vesicles is also quite broad, with various shapes, size, and multilaminar structures formed simultaneously despite the monodispersity of the repeating unit.

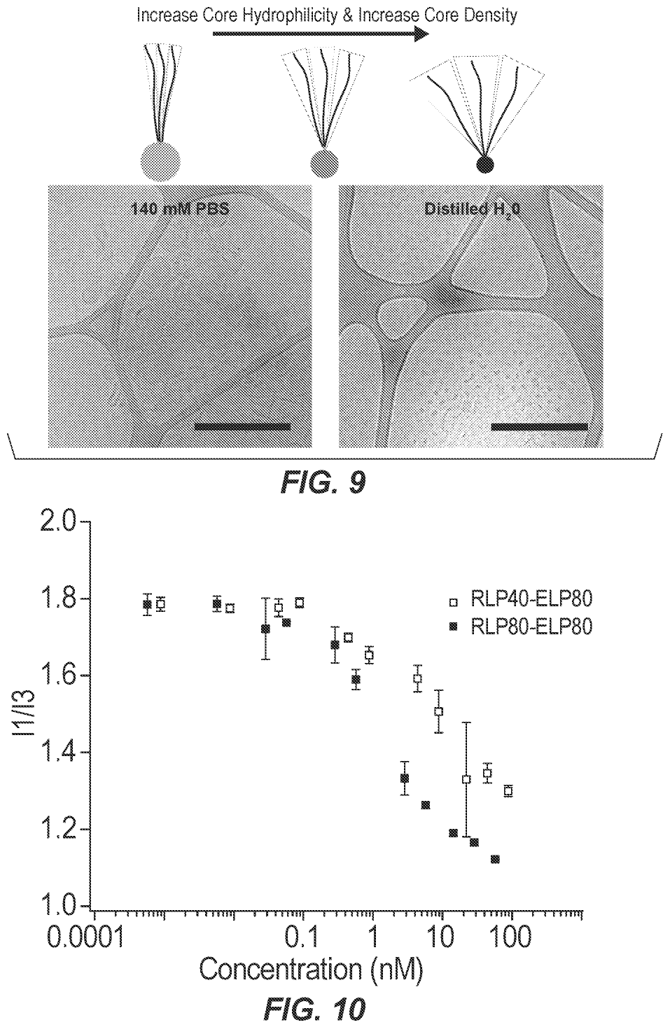

In addition to changes to the block architecture, one can affect the assembly of these dynamic molecules but affecting the solvent quality. A dramatic example of this affect can be seen in FIG. 9, where modifying the buffer of (GRGDSP[Y:V]S)80-ELP80 (SEQ ID NO: 110) from 140 mM PBS to water—a poor solvent for the core and superior solvent for the corona—changes the assembly from vesicular/worm-like micelles to spherical micelles. This is further evidence to support our hypothesis that changes to the chemical sequence affect the chain volume and thus changes in hydrophobicity also result in physical changes at the chain level.

All of these modifications to the core and corona chains prompted us to measure the critical micelle concentration (CMC) of these micelle constructs. Thus, we employed a well-developed technique in the laboratory for measuring the CMC-encapsulating pyrene in the core of the particle. Depending on the polarity of the solution, pyrene will exhibit a different fluorescent signal of peaks 1 and 3 (11 and 13). Upon a concentration above the CMC, pyrene will be sequestered by the core of the particle, modulating this ratio of 11 and 13. For RLP40-ELP80 (SEQ ID NO: 84) and RLP80-ELP80 (SEQ ID NO: 91) we approximate a CMC that is between 100 and 500 nM according to the point of decreasing I1/I3 ratio (FIG. 10) which suggests that these micelles are slightly more stable than previously measured block co-polypeptides. Interestingly, this I1/I3 ratio has been tabulated for various solvents suggesting that the polarity of the interior of our particles are closer to that of acetone (1.4) than water (1.8). Thus, we hypothesized that these micelles may be capable of sequestering hydrophobic moieties similar to block co-polymer micelles. To this end, we designed a series of RLP40-ELP80 proteins that were intended to have similar assembly dimensions but different core chemistries. For the core sequence, we chose the repetitive units (GRGDSPYS) (SEQ ID NO: 24), (GRGDSPYQ) (SEQ ID NO: 22) and (GRGDQPYQ) (SEQ ID NO: 20) which all have similar UCST binodal lines but different non-charged polar residues which may form different strength H-bonds with the drug. For the small molecule drug, we chose paclitaxel as it is notoriously insoluble in aqueous solvents and suffers side effects associated with its delivery vehicle.

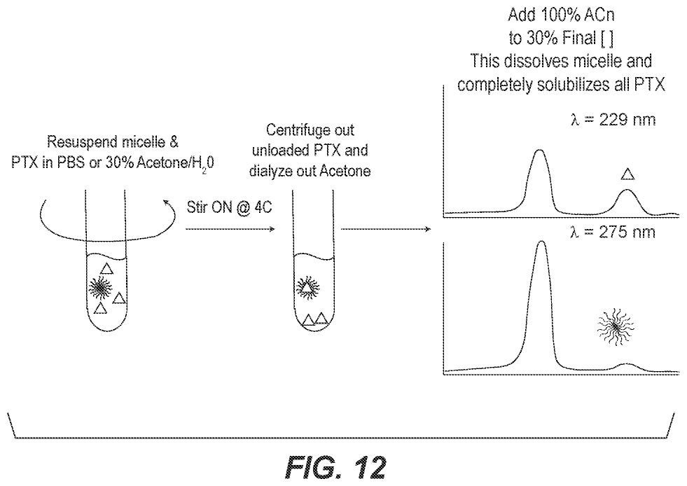

Using these particles, we incubated the polypeptide chains in the presence of 10× molar excess of paclitaxel (PTX) in H₂O or 30% acetone+H₂O mixture overnight at 4° C. to maintain a soluble, assembled micelle in the presence of large excess of insoluble PTX (FIG. 12). We then centrifuged out the still insoluble PTX in the H₂O sample and dialyzed out the acetone into milliQ H₂O, also removing the still insoluble PTX after dialysis by centrifugation. We then added 100% acetonitrile to a final concentration of 30% v/v to completely dissolves the micelle and resuspend the PTX into a homogenous mixture of PTX plus protein. This sample is run on a C₁₂ analytic HPLC column to separate the protein and PTX peaks. Using the relative size of the peaks and the known extinction coefficients of PTX and the protein at 230 nm and 275 nm respectively, we can determine a molar ratio of PTX to protein that remained soluble after we subtract from a no-vehicle control. In essence, this experience tells us how much the solubility of PTX increases in the presence of various micelle systems, with the supposition being that this increase in suspension concentration is due sequestration into the core of the particle.

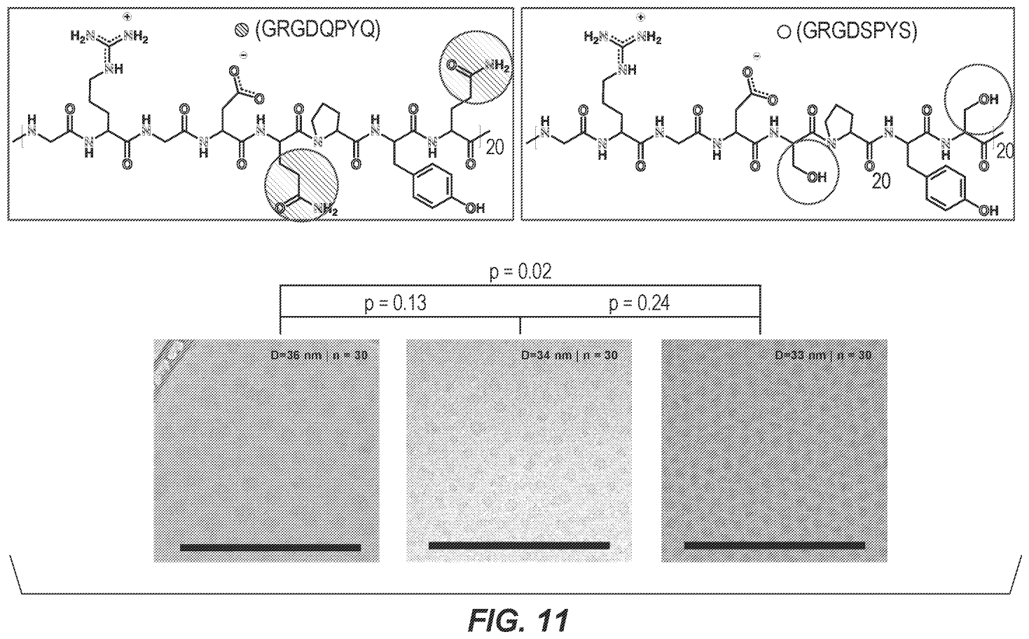

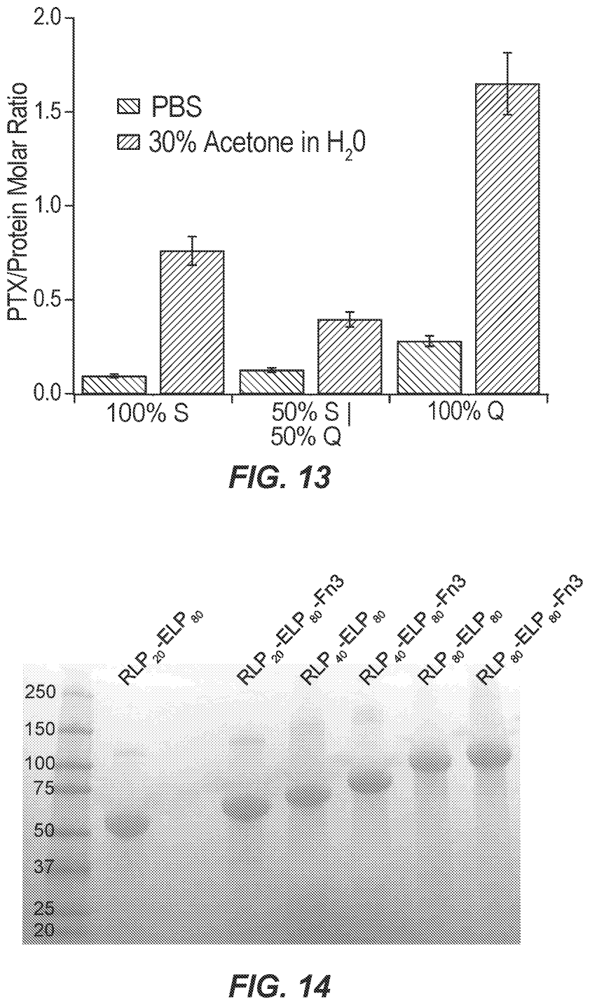

Our results support the conclusion that the various RLP-ELP micelles are capable of increasing the observed solubility of PTX in normally unsuitable solvents. We observe a dramatic increase in loading when using acetone as a cosolvent, perhaps because it can diffuse readily into the core of the particle, which has similar polarity to acetone. We also observed dramatic differences between the subtle particles' chemistries, suggesting that primary amino acid sequence can control this partitioning coefficient by 2 to 3-fold (FIG. 11 and FIG. 13). In our best performing cases, 100% Gln substitution for Ser, we observed PTX/protein molar ratios that correspond to similar conjugation efficiencies achieved with PTX to protein (˜8 PTX per protein chain in 100% Gln substitution case). Thus, we suggest that this physical loading procedure may provide an alternative delivery scheme opposed to direct chemical conjugation to Lys or Cys residues.

### Example 3

Block Co-Polypeptides with UCST and LCST Phase Behavior can be Combined for Multivalent Display of Protein and Peptide Ligands

Using this RLP-ELP block co-polypeptide platform, we sought to develop a micelle capable of multivalent display. We selected a targeting domain, the 10th type III domain from human fibronectin (Fn3) that targets the human αvβ3 integrin, a receptor that is upregulated in the endothelium of many tumors and is overexpressed on several tumor cells such as glioblastoma, renal cell carcinoma, ovarian carcinoma and breast cancer metastases. We chose an Fn3 variant that binds the αvβ3 integrin with low affinity (K_{D}>1×10⁻⁷ M) and can be expressed in E. coli as a fusion to repetitive polypeptides such as ELPs. The low affinity of the parent Fn3 domain is important as, multivalent presentation could amplify its avidity, which may not be possible with ligands that possess intrinsically high affinity, so that we could test for the effect of self-assembly and multivalency on binding avidity and cellular uptake.

After assembly of the genes in the expression vector, each vector was transformed into the BL21(DE3) strain of E. coli and overexpressed by a previously published protocol. The block co-polypeptides were isolated from the soluble fraction of the cell lysate and purified by inverse transition cycling, a non-chromatographic method, to >95% purity as determined by SDS-PAGE (FIG. 14). Yields of all polypeptides were >20 mg·L⁻¹ of shaker flask culture without any optimization of the expression protocol, typical to other Fn3 expression and purification schemes that yield 5-20 mg·L⁻¹.

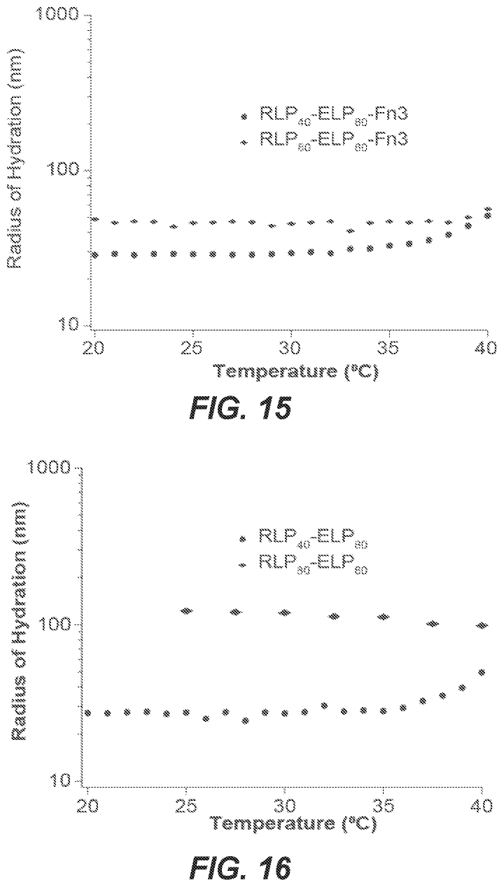

Each block co-polypeptide was analyzed by dynamic light scattering (DLS) at several temperatures between 4° C. and 37° C. to determine the thermal stability of the micelles, and to determine their radius of hydration (Rₕ). The Rₕ of RLP20-ELP80 (SEQ ID NO: 81) and RLP20-ELP80-Fn3 (SEQ ID NO: 95) were ˜7 nm, indicating that these constructs did not assemble within this temperature range and exist as soluble disordered polypeptides, as their Rₕ is similar to that of denatured proteins with a similar molecular weight (Rₕ˜8 nm) and other elastin like polypeptides of similar size. In contrast, RLP40-ELP80 (SEQ ID NO: 84) and RLP40-ELP80-Fn3 (SEQ ID NO: 96) self-assembled into micelles with a Rₕ of 30 and 32 nm, respectively, between 20-37° C. (FIG. 15).

Likewise, RLP80-ELP80 (SEQ ID NO: 87) (112 nm) and RLP80-ELP80-Fn3 (SEQ ID NO: 97) (47 nm) formed stable micelles over the same temperature range (FIG. 16).

Interestingly, the Rₕ of RLP80-ELP80 (SEQ ID NO: 87) is dramatically affected by the presentation of the Fn3 domain on the hydrophilic C-terminal end of the block co-polypeptide (Table 7).

TABLE 7
Light scattering data of Fn3 block co-polypeptides

| Block co-polypeptide | R_{g} (nm) | Rₕ (nm) | N_{agg} | ρ = R_{g}/Rₕ | Shape |
|---|---|---|---|---|---|
| RLP20-ELP80-Fn3 | — | 6.95 | — | — | Unimer |
| (SEQ ID NO: 95) | | | | | |
| RLP40-ELP80-Fn3 | 29.3 | 29.3 | 201 | 1.0 | Spherical |
| (SEQ ID NO: 96) | | | | | |
| RLP80-ELP80-Fn3 | 39.2 | 48.7 | 630 | 0.8 | Sphere & |
| (SEQ ID NO: 99) | | | | | Wormlike |

This result makes sense, as RLP80-ELP80 (SEQ ID NO: 87) exists on the edge of the phase boundary that separates spherical and worm-like micelles. Therefore, it is plausible that the incorporation of a small folded protein could result in a change of shape. It also appears that the Fn3 domain is not stable at temperatures above 37° C., as there is a precipitous increase in the Rₕ of RLP40-ELP80-Fn3 (SEQ ID NO: 96) between 36-40° C. Based on this result, samples were maintained on ice prior to flow cytometry and confocal microscopy.

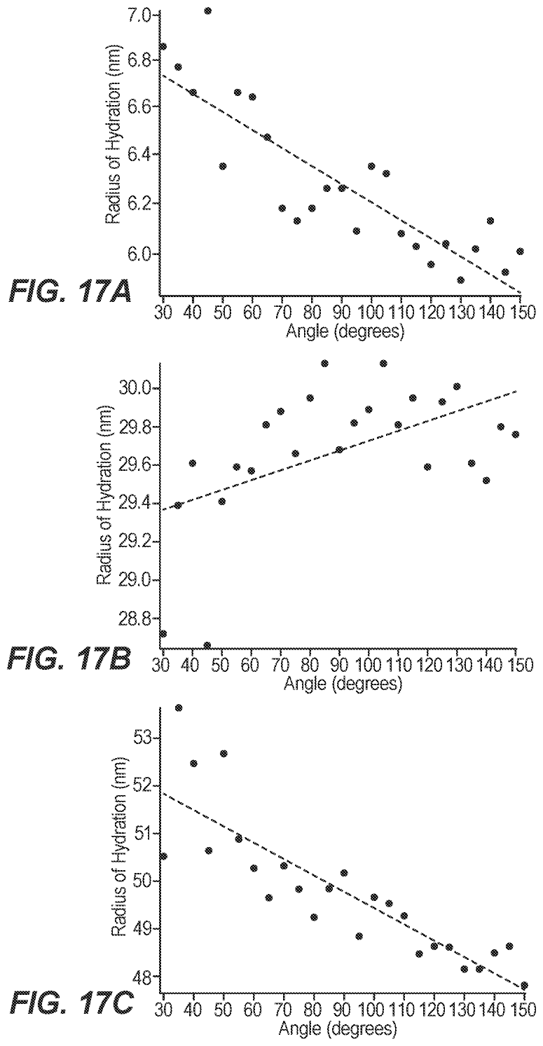

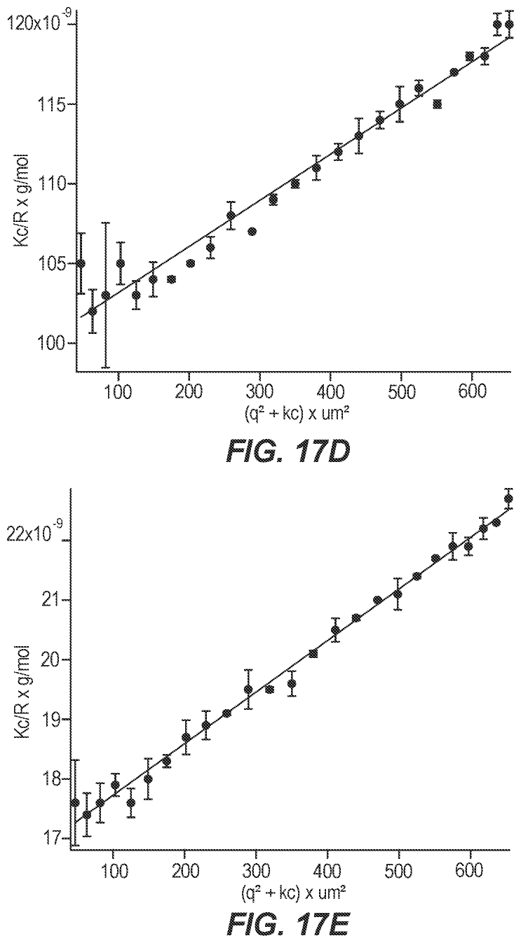

Based on our previous results, we speculated that micelles with a Rₕ in the 30-40 nm range are likely to be spherical, while micelles with a Rₕ >100 nm are likely to be cylindrical or worm-like in structure. To deduce the morphology of these particles and to calculate their aggregation number (N_{agg}), we next carried out static light scattering (SLS) measurements. Increasing the size of the core-forming block from 40 to 80 repeats of (QYPSDGRG) (SEQ ID NO: 1) increases the radius of gyration (R_{g}) from 29 nm to 39 nm, the Rₕ from 29 nm to 49 nm and the N_{agg} from 201 to 630 chains per micelle (Table 7 & FIG. 17).

These results suggest that the larger particles have a higher aspect ratio than the smaller particles. Unfortunately, the SLS results were not conclusive, as the form factor (ρ=R_{g}/Rₕ) did not change dramatically across particles with putatively different morphologies. Typically, p depends on the morphology of the particles with typical values for spheres around 0.7 and increases as the scattering molecule becomes more elongated (i.e. disc shaped, cylindrical structures). Therefore, we next directly visualized the particles with cryo-TEM to confirm their morphology.

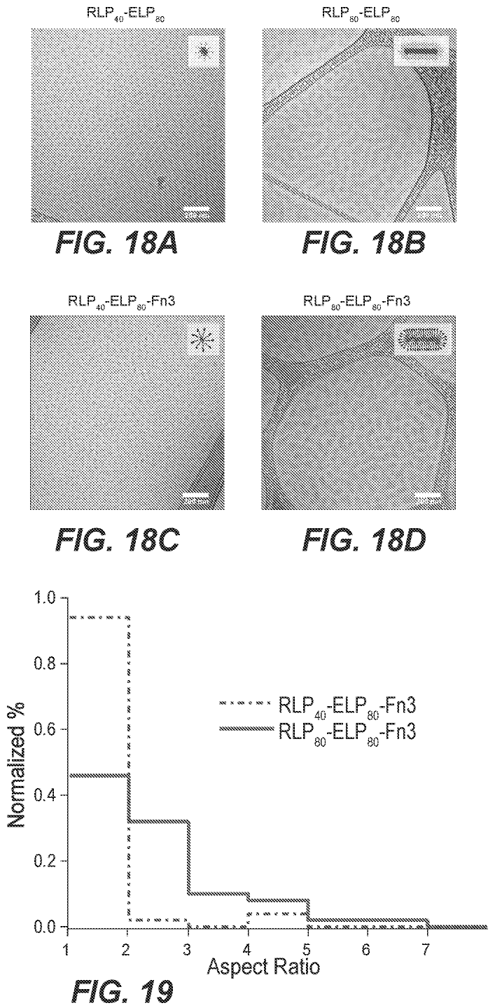

Previous studies of ELP-based micelles have demonstrated that the desolvated core of the micelle can be visualized by cryo-TEM, as it has significant differential contrast than the surrounding water, but the corona is far too solvated to be visualized. Increasing the core-forming RLP block size from 40-80 units without a Fn3 domain (FIG. 18A, B respectively) shifted the morphology from spherical to worm-like micelles, as reported previously. The core of these micelles increased in diameter (Table 8) from 24 nm to 59 nm and the spacing between the particles changed from 27 nm to 42 nm, indicating the elongation of the corona ELP chain. RLPXX-ELP80-Fn3s (SEQ ID NO: 95-97) behave similarly. Increasing the core size increased the core diameter of the micelles from 27 nm to 51 nm and the core spacing from 27 nm to 42 nm (FIG. 18C, D respectively).

TABLE 8
Cryo-TEM core diameter and spacing of RLPXX-ELP80-Fn3s

| | Core Diameter (nm) | Intra-core Spacing |
|---|---|---|
| Block co-polypeptide | [n = 30] | (nm) [n = 30] |
| RLP40-ELP80 (SEQ | 24.3 ± 2.8 | 23.6 ± 4.5 |
| ID NO: 84) | | |
| RLP40-ELP80-Fn3 | 27.9 ± 4.3 | 27.4 ± 4.7 |
| (SEQ ID NO: 96) | | |
| RLP80-ELP80 (SEQ | 39.4 ± 5.4 | 35.0 ± 4.5 |
| ID NO: 87) | | |
| RLP80-ELP80-Fn3 | 44.7 ± 8.0 | 34.4 ± 5.8 |
| (SEQ ID NO: 97) | | |

Image analysis suggests a shift in assembly as RLP40-ELP80-Fn3 (SEQ ID NO: 96) has >90% of particles with an aspect ratio <2 while RLP80-ELP80-Fn3 (SEQ ID NO: 97) has 45% of micelles with an aspect ratio <2 (FIG. 19).

These results both indicate a shift in morphology from spherical to a mixture of spherical and worm-like micelles. These results also corroborate the DLS and SLS experiments measurements indicating that increasing the core block length elongates the micelle morphology, increasing the density of chains in the corona and maintaining the overall shape of the parent block co-polypeptide.

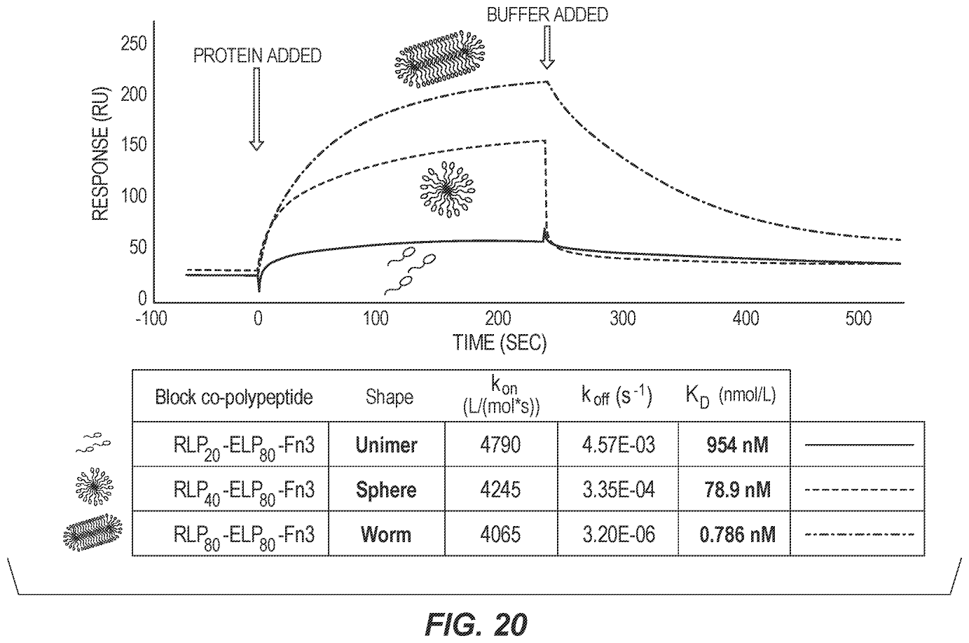

We next used surface plasmon resonance (SPR) to characterize the avidity of the RLPXX-ELP80-Fn3 (SEQ ID NO: 95-97) fusions to the ectodomain of human αvβ3 integrin. SPR sensorgrams were generated for binding of the Fn3-functionalized RLP-ELP80 block co-polypeptides at concentrations ranging between 2.5 and 10 μM. Kinetic association and (kₒₙ) and dissociation constants (k_{off}) are summarized in FIG. 20. As the core block size increases, the kₒₙ increases in magnitude, and the k_{off} decreases in magnitude, both consistent with the increase in size of the binding unit (unimer or larger diameter micelle). As seen in previous studies, Fn3-decorated spherical micelles showed a 10-fold increased avidity for the αvβ3 integrin compared to the RLP20-ELP80-Fn3 (SEQ ID NO: 95) construct that does not self-assemble and hence only presents a single copy of the Fn3-domain. Interestingly, elongating the particle from a spherical to worm-like geometry can increases the avidity for the integrin by ˜1000-fold compared to the monomer ligand, driving avidity into picomolar concentrations (FIG. 20). This result is remarkable when one considers the unoptimized nature of the Fn3, which has K_{D} in the micromolar range for the αvβ3 integrin. The effective K_{D} of the RLPXX-ELP80-Fn3 worm-like micelles is in fact is many orders of magnitude lower than a clinically relevant therapeutic antibody-LM609—which has a K_{D} of ˜20 nM. For context, these binding constants are at the upper threshold of antibodies that are used for targeted cancer therapy targeting, highlighting their clinical relevance.

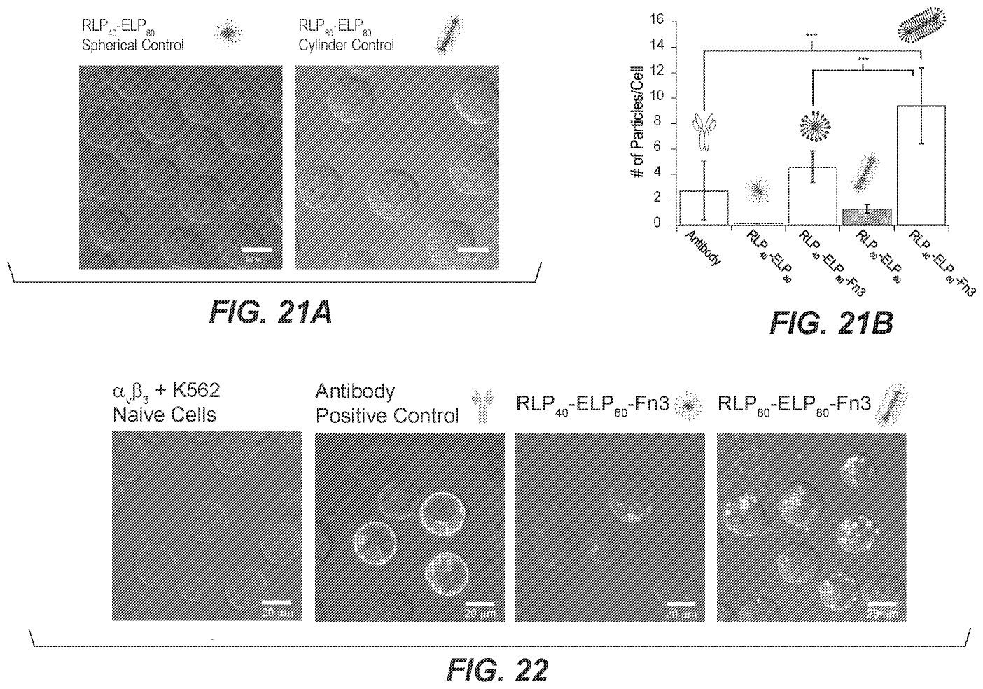

To assess the intracellular uptake of these particles, we used a cell line stably transfected with the αvβ3 integrin. The native cell line, K562, has endogenously low levels of expression of this receptor and therefore serves as the receptor-negative control, and the un-decorated RLPXX-ELP80 micelles serve as ligand-negative controls for each type—size and shape—of micelle. Cells were incubated for 2 hours with a 10 μM solution of various block co-polypeptides at 37° C., a concentration that is well above the CMC and K_{D} of all micelles. Confocal microscopy was first used to study the internalization of the block co-polypeptides by the αvβ3 integrin transfected cell line. Ligand-negative spherical micelles showed low levels of uptake, while that of ligand-negative worm-like micelles was slightly higher (FIG. 21A), consistent with previous observations that shape plays a role in controlling non-specific uptake of nanoparticles.

However, far more dramatic differences were seen for Fn3-decorated micelles. Compared to the parent spherical micelle which showed low levels of uptake (FIG. 21A) that was barely above that of the autofluorescence of the WT—untransfected—cell line, presentation of the Fn3 domain on the RLPXX-ELP80 block co-polypeptide that forms spherical micelles significantly increased its uptake (FIG. 22) as quantified by the number of particles inside the cell membrane (FIG. 21B) and the mean fluorescence of the cell—(p<0.001 unpaired student's t-test).

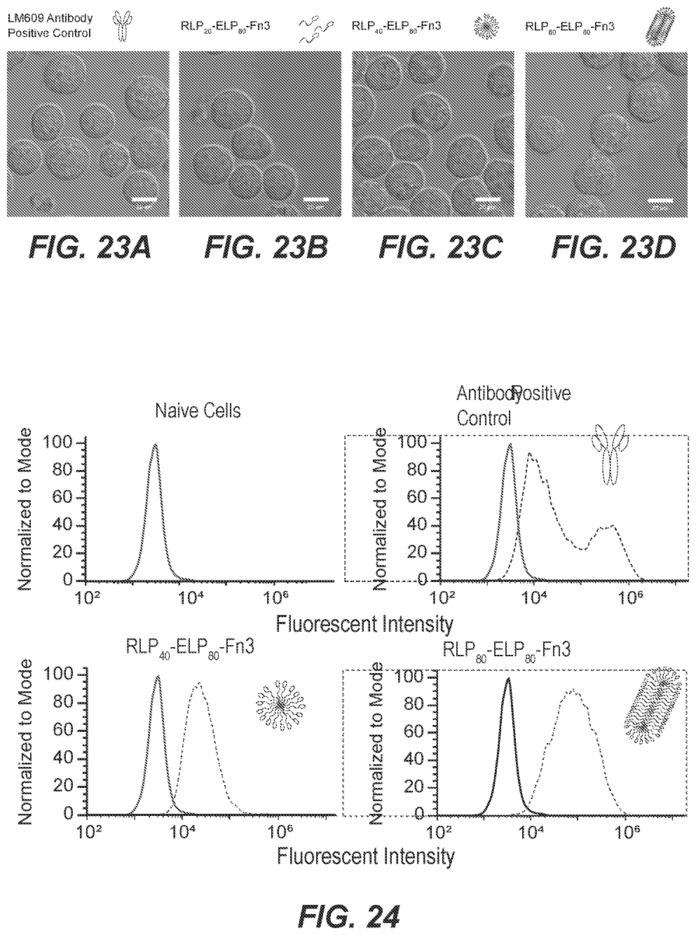

The worm-like micelles that are decorated with the Fn3-ligand similarly showed a much greater level of cell uptake compared to the parent worm-like micelles (FIG. 22). In contrast, without overexpression of the αvβ3 integrin on K562 cells, there were low levels of internalization and uptake of the spherical and Fn3-decorated micelles, showing that most of internalization of ligand-decorated micelles is driven by ligand-receptor engagement (FIG. 23).

The LM609 antibody showed completely different cell uptake than RLP-ELP80-Fn3 micelles. Although it has a high level of fluorescence (FIG. 22), much of the fluorescence was localized at the cell membrane and far lower levels of intracellular fluorescence, especially compared to the Fn3-decorated micelles, indicating that this antibody-integrin binding event does not trigger internalization.

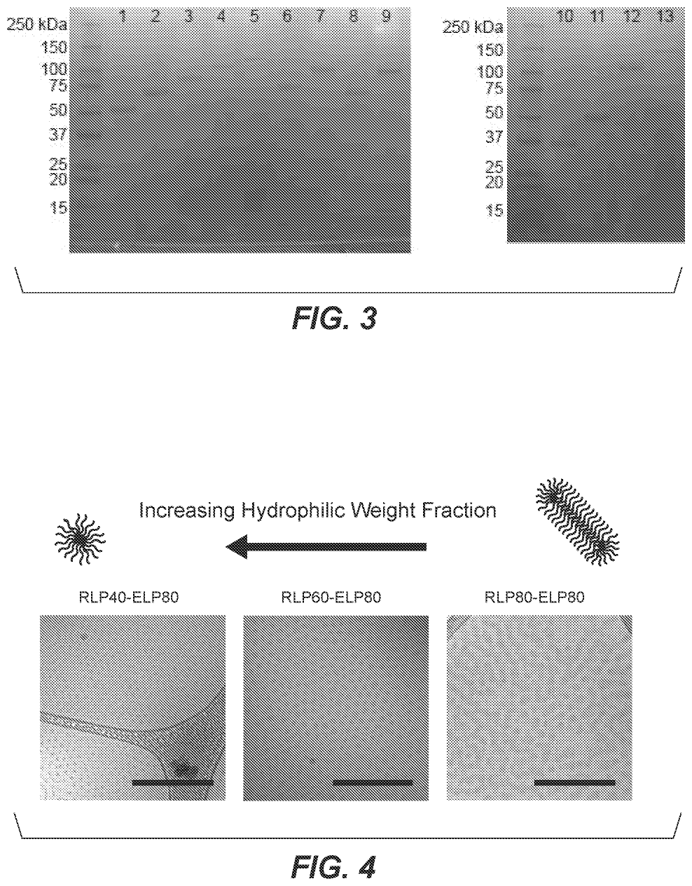

Flow cytometry was next used to quantify the cell uptake. Unstained K562 cells had a background cell fluorescence of 2912 & 3236 (Geometric Mean±StDev) (FIG. 24) which increased to 8686±8787 when incubated with RLP40-ELP80 (SEQ ID NO: 84) spherical micelles and to 24904±13884 for the RLP80-ELP80 indicating that there is a low level but shape-dependent non-specific uptake of the micelles (p<0.001, unpaired student's t-test) (FIG. 3B).

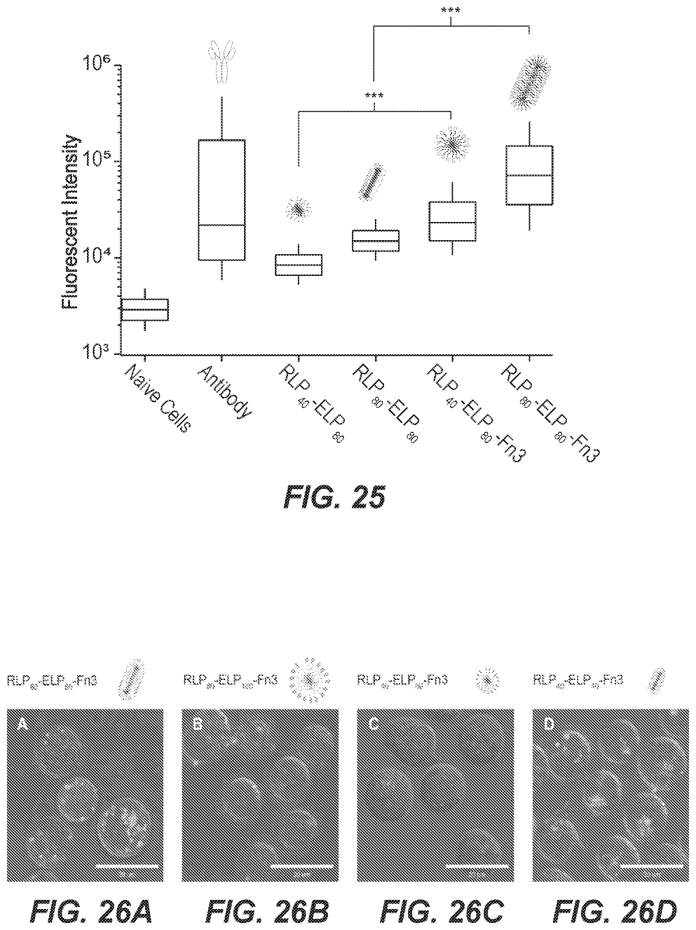

The positive control, the LM609 antibody had a statistically significantly higher uptake of 36708±255175, consistent with its known specificity for the αvβ3 integrin (FIG. 25). A closer look at the flow cytometry data indicates that there is a high-level and low-level receptor expressing cell population, as seen by the two distinct peaks in FIG. 24. Interestingly, spherical micelles formed by RLP40-ELP80-Fn3 (SEQ ID NO: 96) and worm-like micelles formed by RLP80-ELP80-Fn3 (SEQ ID NO: 97) have higher geometric fluorescent intensity means of 15539±286229 and 71382±251919 that are 2-fold and 3-fold greater than the undecorated controls (FIG. 24).

Clearly receptor-mediated endocytosis is shape dependent, as seen by the significantly higher cell uptake exhibited by the worm-like micelles compared to spherical micelles that is consistent with their higher avidity for the integrin. The Fn3-decorated spherical and worm-like micelles also only exhibited a single flow cytometry peak, unlike LM609 that has a bimodal distribution of cell uptake. This result implies that high valency micelles are not sensitive to the heterogeneity of receptor expression, presumably as long as the receptor expression is above a certain threshold to enable multiple ligands to engage the receptors on the cell surface. High valency micelles may therefore provide more robust strategy to target cells with inhomogeneous levels of receptor expression than antibodies.

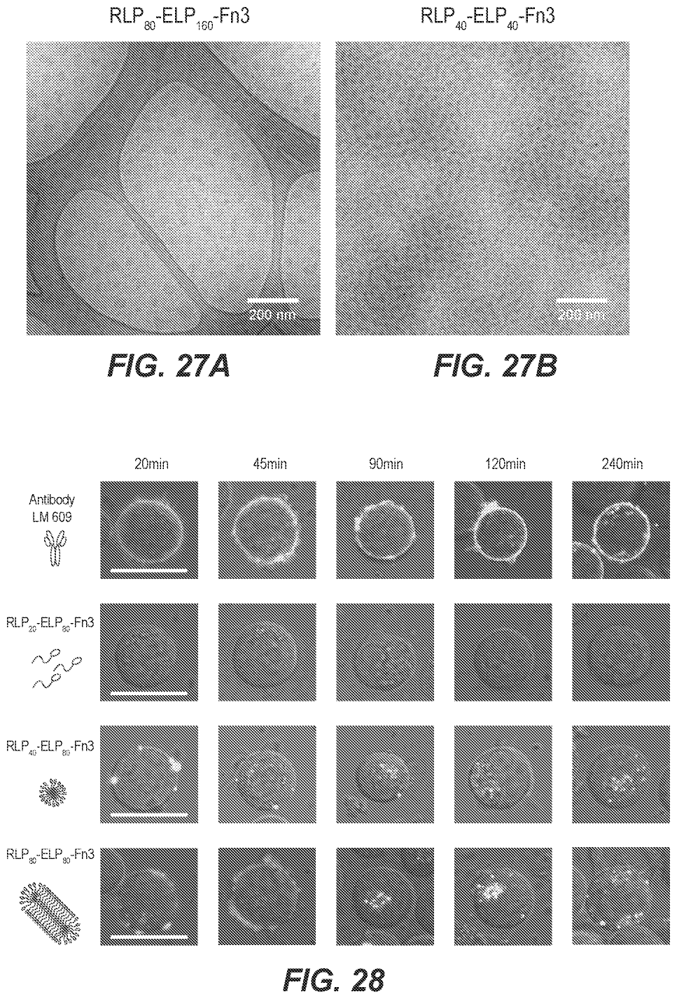

We believe that morphology is more important than size, as worm-like micelles with the same hydrophilic weight fraction as RLP80-ELP80-Fn3 (SEQ ID NO: 97), but that are smaller in size, exhibit higher levels of cell uptake than a spherical micelle of comparable size-RLP40-ELP80-Fn3 (SEQ ID NO: 96) (FIG. 27). Likewise, spherical particles of a similar size than the worm-like micelle of RLP80-ELP80-Fn3 (SEQ ID NO: 97) exhibit very low levels of uptake (FIG. 26). These data indicate that the elongated shape and flexibility of the worm-like micelles increased the number of accessible Fn3 ligands available to bind the receptor.

We next decided to visualize the kinetics of internalization by imaging the cells at 20, 45, 90, 120, and 240 min post-incubation (FIG. 28). Particle and area analysis of the Alexa488 dye was performed on all cells in the visual field for at least 3 separate images resulting in ˜50 individual measurements for each sample. The analysis area was gated to exclude the cell membrane to eliminate non-internalized areas of fluorescence.

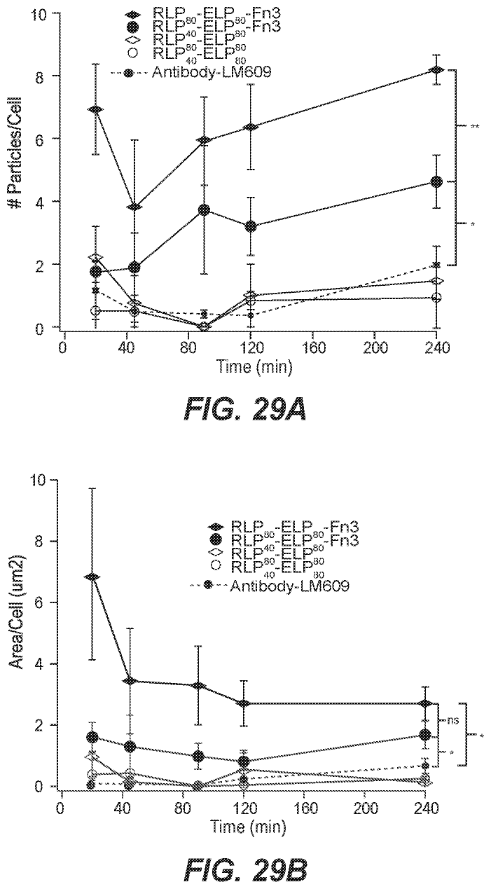

Compared to the LM609 antibody, that remains largely associated with the cell membrane, with only a few isolated fluorescent puncta within the cell at later points, spherical (RLP40-ELP80-Fn3) (SEQ ID NO: 96) and worm-like micelles (RLP80-ELP80-Fn3) (SEQ ID NO: 97) are internalized faster, resulting in more particles within the cell at all time points (FIG. 29A).

Using a 3-way ANOVA for time, shape, and decoration state (Fn3 t) we observed a main effect of shape, decoration state, and time for both the number of particles and the area that they cover in the cell. Pairwise interactions indicate that there are significant effects of micelle shape on the number of particles per cell between spherical and worm like micelles (p<0.01) and spherical micelles and the positive-antibody-control (p<0.05). There were significant differences in the area these particles occupied in the cell between spherical micelles and the antibody control over time (p<0.05) and worm-like micelles and the antibody control (p<0.05) over time (FIG. 29B). There was not a significant difference in particle area between spherical and worm-like micelles over time. The overall percentage of the cell population that contained fluorescent signal was evaluated but did not produce a significant effect over time. The only significant effects of this ANOVA analysis were pairwise effects of micelle shape over time (p<0.001) and decoration state over time (p<0.01). In summary, these data show that: (1) there is a statistically significant increase in cellular uptake with respect to particle morphology (2) a statistically significant increase in cellular uptake by micelles that present an integrin-binding Fn3 domain.

### Example 4

### Two UCST Protein Blocks Resulting in Nano-Scale Self-Assembly

Considering a large library of RLPs previously generated and the vast difference in transition temperatures observed, we set out to create the first block co-polypeptide made of two UCST blocks. From our previous work with ELP block co-polypeptides and RLP-ELP block co-polypeptides we know that there must be a large difference in transition temperatures of the two blocks as each block will influence the other, driving the transitions of each individual block closer together. To begin, we fused the two RLPs with a large difference in Tₜ, (GRGDSPYS) [S], [S]-40, 80 (SEQ ID NO: 100-101) and GRGDQPHN ([QHN]-40). We also varied the block length of the core forming block, as previous experiments demonstrated the importance of the overall hydrophilic weight fraction on the assembled morphology. We also varied the core block by changing GRGDQPHN to GRGDNPHQ ([NHQ]-40) (SEQ ID NO: 102-103). This is a hydrophobic change but maintains the same overall composition of the polypeptide.

The first notable observation is that the choice of these two RLP sequences resulted in variable self-assembly. RLPSS-40-RLPQHN-40 (SEQ ID NO: 100) assembled into an identifiable nanoscale morphology with a R_{g} of 35.1 nm, Rₕ of 28.4 nm, and a N_{agg} of 43 (Table 9). The form factor (R_{g}/Rₕ) suggests that [S]-40-[QHN]-40 (SEQ ID NO: 100) assembles into worm-like micelles. Increasing the molecular weight of the core block increases the R_{g}, Rₕ, N_{agg} dramatically. [S]-80-[QHN]-40 (SEQ ID NO: 101) has a form factor >1 which indicates that [S]-80-[QHN]-40 (SEQ ID NO: 101) likely assembles into worm-like micelles.

TABLE 9
Light scattering characterization of [S]-XX-[QHN]-40 and [S]-XX-
[NHQ]-40 co-polypeptides

| | | MWg · | | | | |
|---|---|---|---|---|---|---|
| Block | Hydrophilic | mol⁻¹ | | | | |
| co-polypeptide | Fraction | chain | N_{agg} | R_{g}(nm) | Rₕ(nm) | ρ |
| [S]-40-[QHN]-40 | 51.5% | 67915 | 43 | 35.1 | 28.4 | 1.2 |
| (SEQ ID NO: 100) | | | | | | |
| [S]-80-[QHN]-40 | 34.6% | 100709 | 1430 | 75.7 | 62.8 | 1.2 |
| (SEQ ID NO: 101) | | | | | | |
| [S]-40-[NHQ]-40 | 51.5% | 67915 | — | — | 6.2 | — |
| (SEQ ID NO: 102) | | | | | | |
| [S]-80-[NHQ]-40 | 34.6% | 100709 | — | — | 7.0 | — |
| (SEQ ID NO: 103) | | | | | | |

Another interesting observation is that the simple change between [S]-XX-[QHN]-40 (SEQ ID NO: 100-101) and [S]-XX-[NHQ]-40 (SEQ ID NO: 102-103) resulted in disassembly. This result was unexpected as the difference in hydrophobicity between [QHN] and [NHQ] is rather small. However, this result can be understood in the sense that this small change narrows the Tₜ gap such that the two blocks have similar enough Tₜ. The similar enough Tₜ between the two blocks results in co-polypeptide behaving as a unimer unit, with just one UCST temperature.

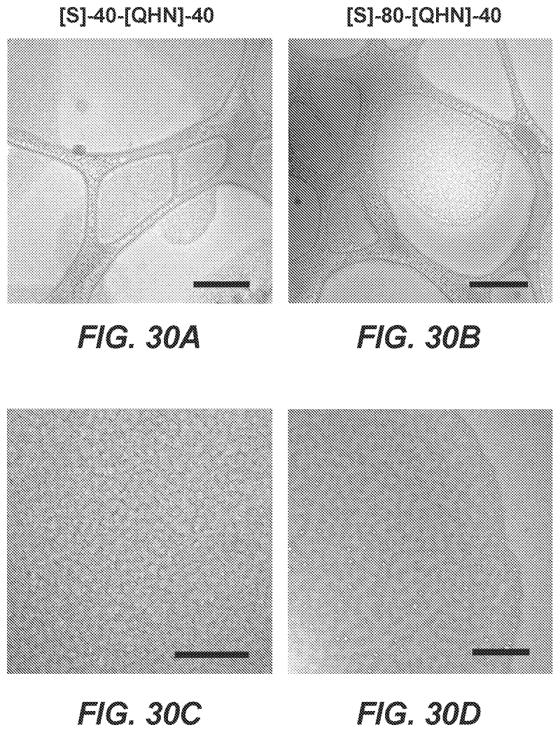

To gain more insight into the morphology of the UCST-UCST constructs, samples were prepared for cryo-TEM. We knew from previous experiments that the vitrification process increases the concentration of the solution. In these images, we observe that the sample polypeptide has undergone liquid like phase separation due to the concentration increase. This phase separation makes it impossible to discern the nanostructure of the two constructs. However, what is interesting is that within the liquid like droplet, there appears to be an interconnected structure (FIG. 30). There also appears to be three distinct areas of contrast—the water/buffer that surrounds the droplets, the contrast of the droplets themselves and then the further contrast of the internal microstructure. This indicates that different RLPs have different contrast upon their phase transition, indicating that RLP-ELPs and RLP-RLPs with different RLPs at the core, may have measurable differences in core contrast. Both [S]-40-[QHN]-40 (SEQ ID NO: 100) (FIG. 30A, C) and [S]-80-[QHN]-40 (SEQ ID NO: 101) (FIG. 30B, D) form this microstructure.

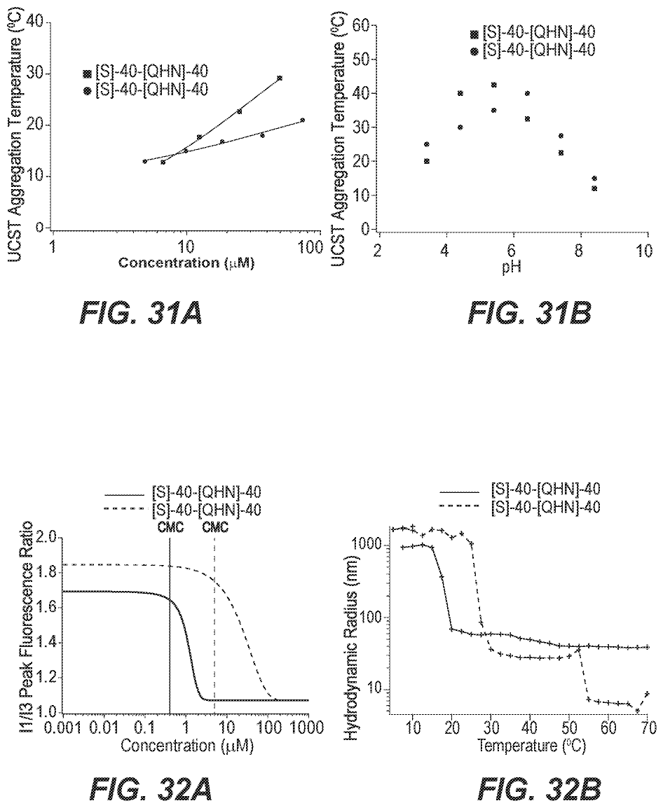

As previously, the temperature dependent turbidity was determined with UV-Vis spectrophotometry. Utilizing this method, we were unable to visualize a clear soluble to assembled transition upon cooling. Additionally, both [S]-40-[QHN]-40 (SEQ ID NO: 100) and [S]-80-[QHN]-40 (SEQ ID NO: 101) had clear UCST aggregation temperatures. For both we determined the concentration dependence of this UCST aggregation (FIG. 31A). Both constructs have much higher UCST values than would be predicted by just the corona block alone, indicating that the fusion to the more hydrophobic RLP brings the observed UCST behavior somewhere between the two unimer blocks alone. Increasing the size of the RLP increased the UCST, indicating that the corona is sensitive to the size of the attached core polypeptide. In comparison to RLP-ELP block co-polypeptides, RLP-RLP block co-polypeptides retain much of their concentration dependence which explains our cryo-TEM results. It is also interesting that [S]-80-[QHN]-40 (SEQ ID NO: 101), which would be predicted to be the more worm-like of the two, has higher concentration dependence. This is also different from the previously observed trend with RLP-ELP block co-polypeptides.

RLP-RLP block co-polypeptides retain the unique pH responsiveness of the corona unimer. Temperature dependent DLS measurements in different buffered pH conditions demonstrated a maximum in the observed UCST aggregation temperature, again around the isoelectric point of His (FIG. 31B). Both constructs UCST increases in almost a linear fashion from pH 8.4 to pH 6.4 and then decreases from pH 6.4 to 3.4. In the context of what was observed earlier, this result makes sense. As the pH decreases towards the isoelectric point of His, there is an increase in the T, because the corona is behaving more hydrophobic. Although this process is not understood, it remains consistent with previous observations. After reaching the isoelectric point, the Tₜ decreases because the massive amount of positive charge in the corona chain which would dramatically increase chain repulsion. It is important to note, that in each of these aggregation DLS curves, that a stable micelle regime was observed. [S]-40-[QHN]-40 (SEQ ID NO: 100) did not appear to have a large change in Rₕ as the pH decreased to the isoelectric point indicating that there was likely not a change in the morphology of the structure. [S]-80-[QHN]-40 (SEQ ID NO: 101) did exhibit a size change as the pH decreased towards the isoelectric point and then actually dropped below the original size measured at pH 7.4 at pH 3.4. This would indicate a morphology change from worm at neutral pH to a more elongated worm at pH 5.4 and then possibly to a sphere at pH 3.4.

Our UV-Vis measurements did not answer important questions about the temperature dependent disassembly. Therefore, to get a more accurate picture, we monitored the Rₕ as we slowly cooled the solution. We observed two different overall behaviors. [S]-40-[QHN]-40 (SEQ ID NO: 100) upon cooling underwent a clear unimer to micelle transition at ˜55° C. and a clear micelle to aggregate transition at 28° C. (FIG. 32B). A unimer to micelle transition that is temperature dependent is known as the critical micellization temperature (CMT). As we know from before, this UCST aggregation temperature is dependent on the solution concentration. [S]-80-[QHN]-40 (SEQ ID NO: 101) remained a micelle at the highest temperature we could measure with the instrument. Upon cooling the size of the aggregate increased slightly until a clear aggregate transition at 18° C. These two results indicate that controlling the core block sequence provides control over the aggregate UCST and the CMT.

In addition to these block co-polypeptides, we sought to understand the difference in Tₜ required for the assembly of UCST-UCST micelles. Using a library of repetitive IDPs, we fused progressively more hydrophilic blocks to our core sequence [S]-40, by replacing Tyr residues with Val (SEQ ID NO: 104). We know that this is the most efficient substitution, an aliphatic reside for an aromatic residue. Additionally, we know that [S]-40 is above the minimum core block size required for assembly. Thus, first we created three block co-polypeptides with ˜50% hydrophilic weight fraction where the intended corona chain contains the sequences [Y:V]-40 (i.e., 2 repeats of SEQ ID NO: 30), [Y:3V]-40 (i.e., 2 repeats of SEQ ID NO: 28) and [V]-40 (i.e., 2 repeats of SEQ ID NO: 36) and the core is comprised of [S]-40 (i.e., 2 repeats of SEQ ID NO: 24).

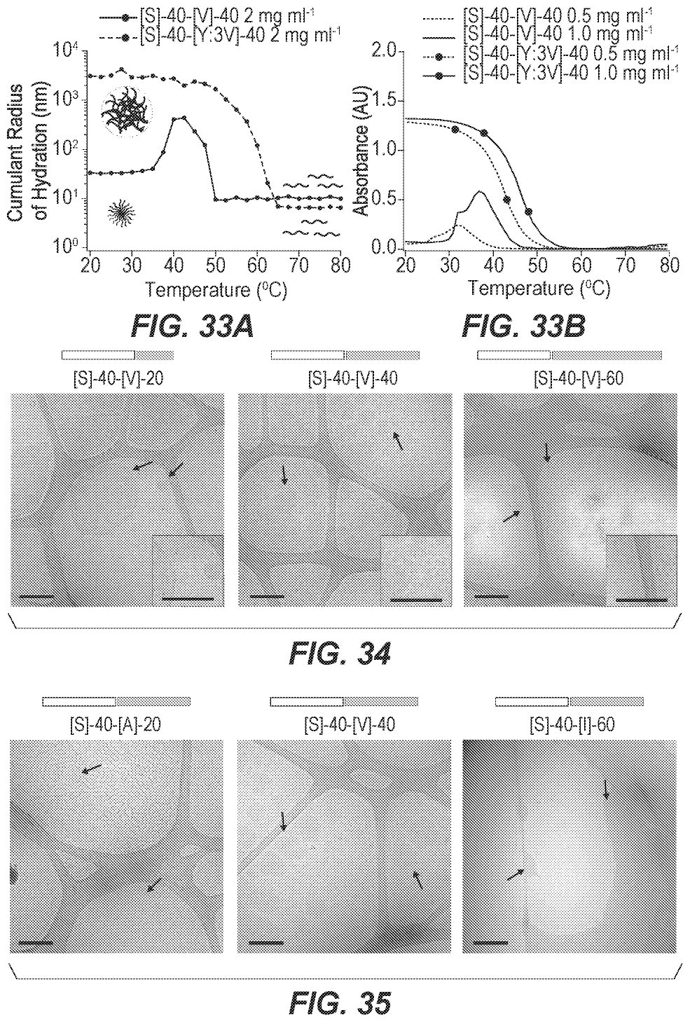

UV-vis spectrophotometry and dynamic light scattering (DLS) measurements were quite instructive in determining the minimum difference required for self-assembly. In these experiments, it became clear to us that coronas comprised of [Y:V] and [Y:3V] only resulted in particulate systems that do not assemble but aggregate in a way approximating liquid-liquid coacervation. Only when Tyr was completely replaced by Val did we observe self-assembled structures. The DLS and UV-vis spectrophotometry show that upon cooling there is an intermediary phase of assembly where the unimer sequences (˜10 nm) transition into ˜500 nm particles before settling into stable 30 nm micelles (FIG. 33). Here, unlike our [S]-40 and [QHN]-40, 80 constructs, we only observe a single mode of assembly with DLS suggesting that the core collapses, but the corona chains remain soluble across the entire temperature range. These results also suggest that a minimum different of the core block is 12.5% with an approximate difference in Tₜ of 80-100° C.

These experiments give us a concept of the core and corona necessary for assembly. From our experiments with UCST and LCST diblocks we know that the relative block sizes influence the assembly of the micelles. Therefore, to investigate the assembly size, we changed the corona size and evaluated their assembly by cryo-TEM. The cryo-TEM images show that our first construct, [S]-40-[V]-40 (SEQ ID NO: 106) assembled into a mixture of small micelles and large phase separated domains. These large phase-separated domains grow as the corona size decreases (FIG. 34). Likewise, increasing the corona size decreases the size of the phase separated domains with a larger percentage of the field of view forming small spherical particles of ˜30 nm Rₕ.

Finally, combining these two insights we made a third systematic library where we change ˜10% of the corona chain with an aliphatic amino acid but one with varying hydrophobicity as predicted by other hydrophobicity scales. Ala, Ise, Val all exhibit similar effects on the UCST phase separation behavior of polypeptides, the only difference between each of these amino acids being extra hydrocarbons on the side group. Thus, we made the proteins [S]-40-[I]-40 (SEQ ID NO: 109) and [S]-40-[A]-40 (SEQ ID NO: 108) in addition to [S]-40-[V]-40 (SEQ ID NO: 106).

These cryo-TEM images suggest that decreasing the hydrophobicity of the chain eliminates the presence of phase separate domains and shifts the assembly phase into a mixture of worm like micelles and spherical micelles, including the presence of a few vesicles. Increasing the hydrophobicity of the corona increases the size and hydrophobicity of the phase separated domains so that they associate with the hydrocarbon grid.

Our employment of a core UCST block was overall very successful in creating predictable self-assembling block co-polypeptides. The avenues of mutation that we pursued were governed by simple principles of diblock assembly from polymer physics and generally resulted in the effects predicted by the theory. Unlike previous protein assembly systems, whose behavior is more difficult to a priori predict, the self-assembly of RLP-ELP block co-polypeptides can be understood by the hydrophilic weight fraction, polypeptide-polypeptide and polypeptide-solvent interactions. This finding is crucial because provides a route for the de novo design of desired nanoscale morphologies from first principles, into a wide variety of shapes and sizes.

Using these block co-polypeptides as scaffolds for assembly, our results clearly show that RLPXX-ELP80 block co-polypeptides are a robust platform for multivalent display of Fn3 domains via self-assembly. The morphology of the parent micelles—spherical versus worm-like—can be tuned by modulating the block ratios and the molecular weight of the core. Decreasing the hydrophilic weight fraction from ˜0.7 to ˜0.46 to ˜0.30 changes the morphology from unimers to spherical micelles to worm-like micelles, respectively. Importantly, the gene-level fusion of Fn3 domain that targets the αvβ3 integrin at the hydrophilic, C-terminal end of the block RLP-ELP co-polypeptide does not abrogate self-assembly and enables the high-density presentation of a Fn3 domain on the corona of the micelles. Fn3 presentation does, however, have an impact on morphology, as the parent micelle, RLP80-ELP80, which exists on the phase boundary between spherical and worm-like micelles, converts to worm-like micelles upon presentation of the Fn3 domain on the corona of the block co-polypeptide.

Cell uptake studies of Fn3-presenting RLP-ELP block co-polypeptides with an αvβ3 overexpressing cell line yielded four notable results: first, compared to the parent-ligand negative micelles—that in the best case—a worm-like micelle—has 3-fold greater cell uptake at 2 hours, demonstrating that multivalency can greatly enhance the targeting potency of a ligand simply by virtue of the avidity effect. Second, compared to a RLPₙ-ELP-Fn3 fusion that does not self-assemble into a micelle and hence only presents a single copy of the Fn3 domain that target the αvβ3 integrin, we found that multivalent spherical and worm-like micelles have an higher avidity and greater cellular uptake, showing the importance of multivalency in amplifying the avidity of a ligand by presentation of multiple copies on a nanoscale scaffold. Third, we observed a dramatic difference in cell uptake as a function micelle morphology, where Fn3-decorated worm-like micelles showed a 5-fold increase in cell uptake compared to spherical micelles. Fourth, we believe that morphology is more important than size, as worm-like micelles with the same hydrophilic weight fraction as RLP80-ELP80-Fn3 (SEQ ID NO: 97), but that are smaller in size, exhibit higher levels of cell uptake than a spherical micelle of comparable size (RLP40-ELP80-Fn3) (SEQ ID NO: 96). Likewise, spherical particles of a similar size than the worm-like micelle of RLP80-ELP80-Fn3 (SEQ ID NO: 97) exhibit very low levels of uptake. These data indicate that the elongated shape and flexibility of the worm-like micelles increased the number of accessible Fn3 ligands available to bind the receptor. Fifth, the avidity and cell uptake of the best performing worm-like micelles is greater than a therapeutically relevant antibody that targets the same receptor.

This class of self-assembling RLP-ELP block co-polypeptides provide an exceptionally robust and versatile system for the molecular design and recombinant synthesis of micelles for delivery of drugs and imaging agents for the following reasons, compared to other ELP-based nanostructures. First, RLP-ELP block co-polypeptides, unlike ELP block co-polypeptides, follow canonical rules of polymer self-assembly via genetic encoded sequences, which make it easier to program their morphology de novo for specific applications. Second, these micelles have significantly greater thermodynamic stability than ELP micelles, as they have CMCs in the ≤0.1 μM range, compared to the 5-10 μM CMC of ELP micelles. Third, these micelles enable presentation of an Fn3 domain on their corona, which is an attractive choice as a targeting ligand, as the Fn3 scaffold is an enormously mutable targeting scaffold and allows variants to be discovered by library screening approaches against diverse targets. Fourth, we note that these targeted micelles can be loaded with drug simply by conjugation of small molecule drugs into the core-forming, hydrophobic domain, in a manner similar to our previous ELP micelles. Finally, their manufacturing—and hence clinical translation—can leverage the bacterial fermentation and downstream purification capabilities of the biopharmaceutical industry.

### Example 5

We wanted to thoroughly investigate the effects of pAzF-introduction as well as nanoparticle crosslinking on nanoparticle self-assembly. For this, we started by characterizing solely our five different ELP/RLP diblock architectures without any C-terminal functionalization. After expression and purification of the diblock constructs using ITC, the pAzF-containing constructs were crosslinked in solution by exposure to UV irradiation and characterized using dynamic light scattering (DLS). The measured hydrodynamic radii (Rₕ) showed that both the introduction of the unnatural amino acid into the polypeptide sequence and particularly the crosslinking process itself did influence the general size of the particles (Table 10). Generally, both processes seem to have increased the measured radii with this effect being more pronounced for the worm-forming constructs.

TABLE 10
Native and Crosslinked Constructs

| | | UAA2-40 | | UAA5-40 | |
|---|---|---|---|---|---|
| | | (SEQ ID NO: 54) | | (SEQ ID NO: 56) | |
| R_{H} (nm) | DB-40 | Native | Crosslinked | Native | Crosslinked |
| 15 μm in PBS | 28.0 ± 1.0 | 28.1 ± 0.2 | 29.8 ± 0.3 | 29.8 ± 0.2 | 34.3 ± 0.3 |
| 15 μm in 7.2M GuHCl | 0.7 ± 1.0 | 7.0 ± 1.1 | 54.1 ± 2.0 | 5.9 ± 1.6 | 52.1 ± 1.0 |

| | | UAA4-80 | |
|---|---|---|---|
| | | (SEQ ID NO: 52) | |
| R_{H} (nm) | DB-80 | Native | Crosslinked |
| 15 μm in PBS | 39.9 ± 0.6 | 55.7 ± 2.3 | 86.2 ± 4.5 |
| 15 μm in 7.2M GuHCl | 1.1 ± 0.1 | 7.4 ± 1.9 | 138.1 ± 12.5 |

Apart from changes in nanoparticle morphology we were particularly interested in the effect pAzF-crosslinking had on their stability. In order to evaluate whether the crosslinking process had indeed led to significantly increased stability, we exposed the particles to guanidine hydrochloride (GuHCl). GuHCl is a well-known denaturing agent that disrupts any inter- and intramolecular electrostatic forces and completely breaks down the quaternary, tertiary and secondary structure of most known proteins. Covalent bonds—such as the ones formed by pAzF-crosslinking—however remain unaffected by GuHCl, thus, the addition of this denaturing agent was expected to give us insights on the stability of our crosslinked nanoparticles. Further DLS experiments proved that crosslinking had indeed yielded the desired stability increase. All crosslinked samples in GuHCl showed Rh values in the same general range as the ones previously measured in phosphate buffered saline (PBS). All native samples on the other hand resorted to complete unimerization upon exposure to GuHCl. As the Rₕ values for the crosslinked particles in GuHCl were generally larger than in PBS, the DLS data furthermore indicates a swelling behavior in the presence of the denaturing agent. This can most probably be attributed to the RLP core which is assumed to be completely collapsed in PBS but will however try to reach an elongated, random-coil morphology in the presence of GuHCl.

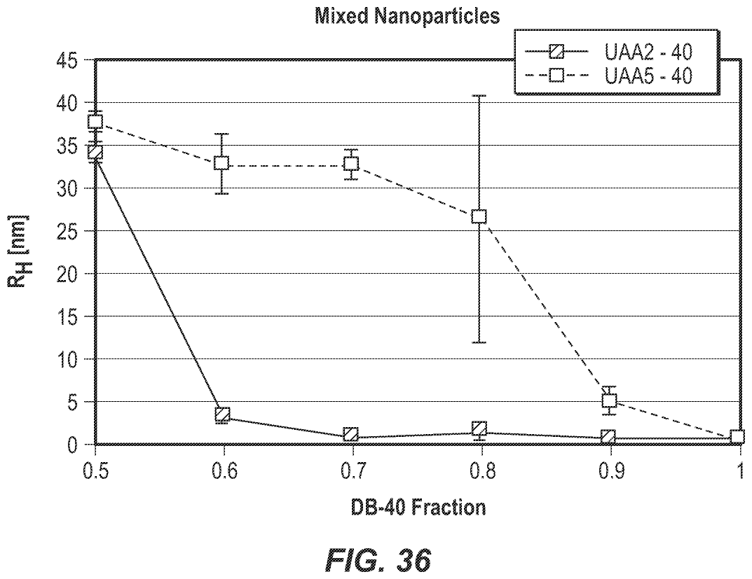

Lastly, the dataset also showed that the UAA2-40 and UAA5-40 constructs are identical both with respect to particle morphology as well as stability. It seemed that two pAzF residues per polypeptide chain is sufficient to achieve stable crosslinking. To determine the minimal pAzF density required for stable crosslinking we mixed the UAA2/5-40 constructs with the pAzF-free DB-40 diblock and crosslinked them at a DB-40 fraction of 50, 60, 70, 80 and 90 percent. The following DLS characterization in 7.2 M GuHCl then showed that the cutoff lies at around one pAzF residue per ELP/RLP chain which in principle is a very intuitive result (FIG. 36). Though the average DLS readings seemed to indicate that this is a rather abrupt transition, closer examination showed that this is not actually the case. In the range of one to two pAzF sites per diblock we also observed a secondary population corresponding to the unimer fraction which continuously decreases in size as the pAzF density increases.

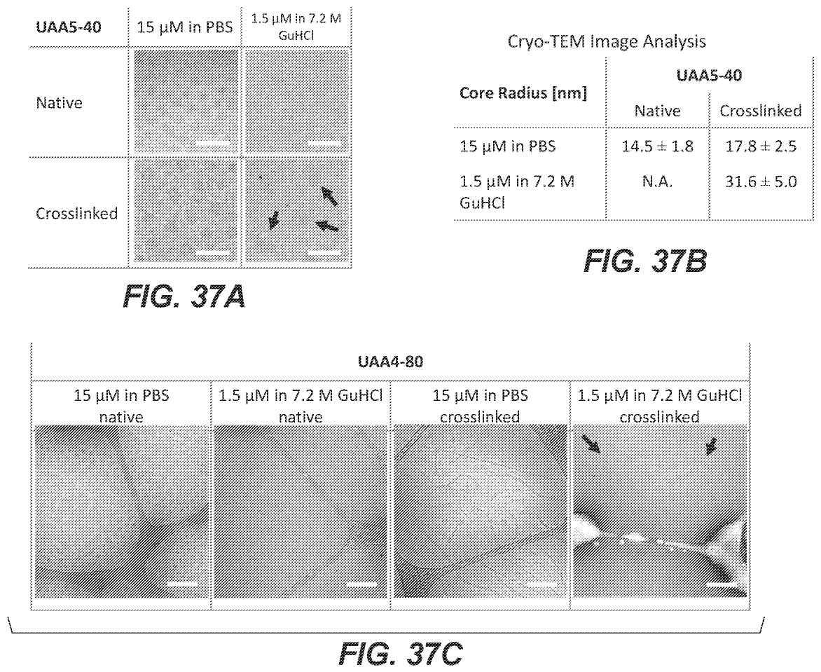

As DLS only provides insights about the general size of nanoparticles but does not tell us anything about potential changes in morphology from one sample to the other, we decided to also perform cryogenic transmission electron microscopy (cryo-TEM) imaging to image our particles in near-native conditions. For the sphere-forming diblock architectures (UAA5 variants), cryo-TEM supports our light scattering data: spherical particles which slightly increase in size upon exposure to GuHCl in the crosslinked regime and completely disassemble without prior crosslinking (FIG. 37). Moreover, the images also confirm the previous observation that the crosslinking process by itself already leads to a significant increase in particle size. The image analysis then resulted in particle radii significantly below the DLS values (FIG. 37C). This can be explained by the previous observation that only the collapsed micelle cores have a high enough electron density to be imaged by TEM. By subtracting the measured core radii from the corresponding Rh values from DLS we also see that indeed the changes in particle size upon GuHCl exposure stem mostly from swelling of the RLP core rather than the ELP corona.

The cryo-TEM images of the worm-forming constructs showed a rather unexpected situation with worms several micrometers in length after crosslinking (FIG. 37C). The particles in the image taken under native conditions were not nearly as elongated as the ones in the crosslinked state suggesting an artifact of reorganization of particles at the grid surface upon deposition. This data indicates that the pAzF crosslinking can occur on a timescale greater than that of polypeptide rearrangement, allowing the particle to adopt new morphologies in response to the crosslinks being formed. In either case, these elongated structures were also robust to denaturing conditions as for their spherical counterparts.

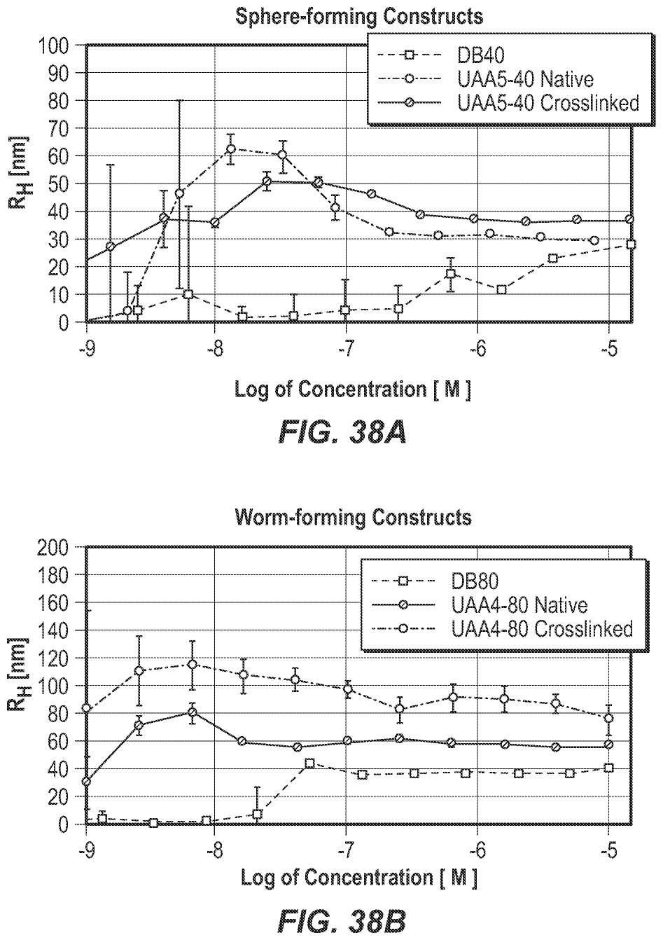

Lastly, we also wanted to determine the critical assembly concentration (CAC) of the individual constructs. The Rₕ values were measured with DLS over a dilution series from the mid-micromolar down to the low nanomolar range. The resulting hydrodynamic radii indicated that the sphere-forming constructs generally are more stable than their worm-forming analogues with the DB-40 and DB-80 constructs disassembling at low micromolar and mid-nanomolar concentrations respectively (FIG. 38). Curiously, the mere introduction of crosslinking sites seems to dramatically decrease the CAC. Generally, the worm-forming constructs had lower CACs than their spherical analogues and so do pAzF-containing constructs in comparison to analogous pAzF-free polypeptides.

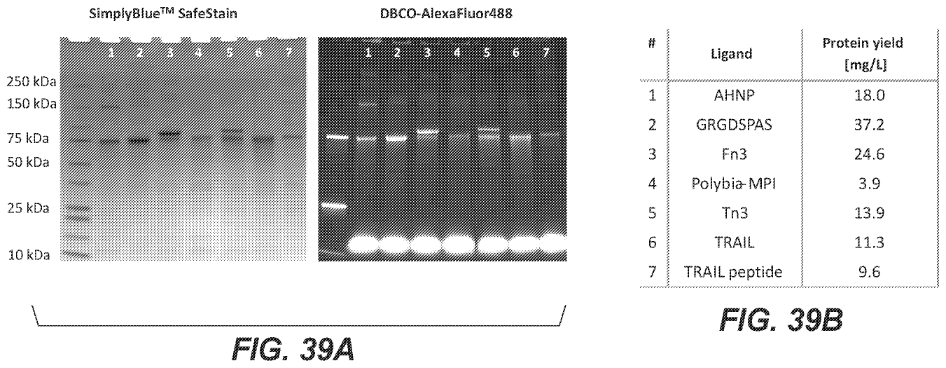

To create a particle that can target a specific cellular receptor, protein ligands were genetically fused to the outside of our crosslinkable particles. Our nanoparticle-ligand fusions display remarkably strong expression compared to other un-natural expression systems (FIG. 39A, B). All but one culture had produced around or above 10 milligrams per liter of liquid culture.

The first step of the characterization process was to produce crosslinked particles for the functionalized diblock constructs and to analyze whether the attachment of the ligands had caused any significant changes in particle size. The following DLS analysis showed that the functionalization did not have any substantial influence on most of the nanoparticle architectures (Table 11). The two exceptions were the constructs carrying the AHNP and TRAIL peptide ligands which showed significantly increased and decreased hydrodynamic radii respectively. The most plausible explanation is that this was caused by the decreased solubility observed for both these constructs which might have significantly altered the actual concentrations in solution. Whatever the reason was, it had no effect on the crosslinking process as the particles still remained stable after GuHCl exposure.

TABLE 11
Crosslinked UAA5-40-K8D4-Ligand Constructs

| | R_{H} (nm) | |
|---|---|---|
| Ligand | 7 μM in PBS | 0.7 μM in 7.2M GuHCl |
| Unfunctionalized UAA5-40 | 34.3 ± 0.3 | 52.1 ± 1.0 |
| (SEQ ID NO: 56) | | |
| AHNP (SEQ ID NO: 74) | 46.7 ± 0.4 | 51.4 ± 0.8 |
| GRGDSPAS (SEQ ID NO: | 33.9 ± 0.5 | 48.7 ± 0.7 |
| 76) | | |
| Fn3 (SEQ ID NO: 60) | 37.6 ± 1.2 | 46.4 ± 0.6 |
| Poly BIA-MPI (SEQ ID NO: | 33.8 ± 0.4 | 44.0 ± 1.1 |
| 78) | | |
| Tn3 (SEQ ID NO: 62) | 32.3 ± 0.4 | 47.8 ± 0.7 |
| TRAIL peptide (SEQ ID NO: | 27.6 ± 6.8 | 47.5 ± 6.7 |
| 70) | | |

To determine the effect of crosslinking on ligand uptake, a series of cell experiments were performed. More concretely, the experiments were performed on four different cell lines depending on the type of ligand: The colorectal cancer cell line Colo205 was used for the apoptosis-inducing DR5-targeting ligands (Tn3 and TRAIL peptide), the breast cancer cell line SK-BR-3 was chosen to determine the potency of the ErbB2-binding AHNP ligand and two different variants of the leukemia cell line K562 (native and transfected with the gene for αvβ3-integrins, see Dzuricky et al.) were employed to characterize the integrin-targeting constructs. The leukemia cell lines were also used to test polybia-MPI as its cytotoxicity had been reported for K562 cells 98.

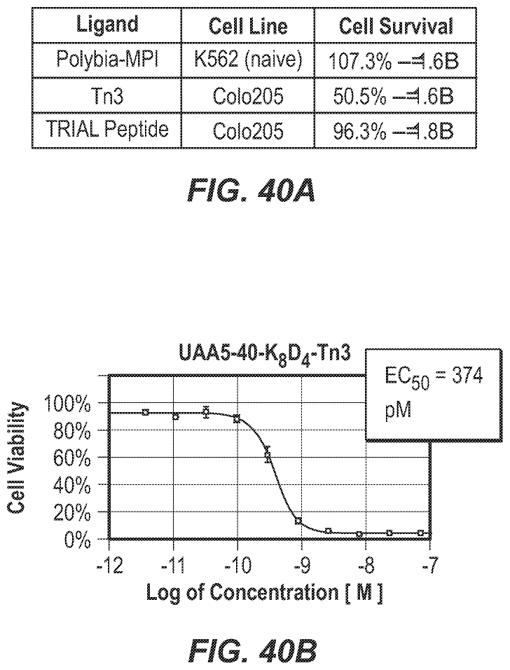

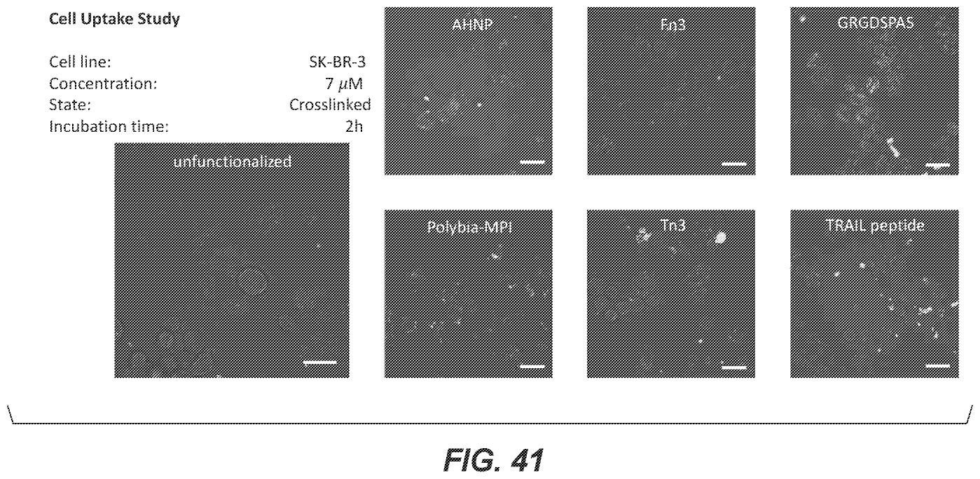

The potency of the three cytotoxic ligands Tn3, TRAIL peptide and polybia-MPI was evaluated by performing cell viability assays on the respective cell lines at different concentrations. The subsequently collected data then showed that only the exposure to the Tn3-functionalized Nanoparticles had led to any cell death (FIG. 40). For the other two constructs the cells showed complete survival in the investigated concentration range. With an EC50 value of 470 μM the Tn3 sample still induced cell death at concentrations significantly below the CAC of the UAA5-40 construct.

To also investigate possible non-specific uptake, all six ligands plus the unfunctionalized construct were tested on the SK-BR-3 cells. The resulting confocal images after 2 hours of co-incubation with the crosslinked, AlexaFluor-488-tagged nanoparticles at a concentration of 7 μM are shown in FIG. 41. The images clearly showed that the AHNP functionalization did not lead to any increased cell uptake in comparison to the other ligands suggesting that ligands will respond differently to multivalent display and thus engineering multivalent display is critical for engineering functionality.

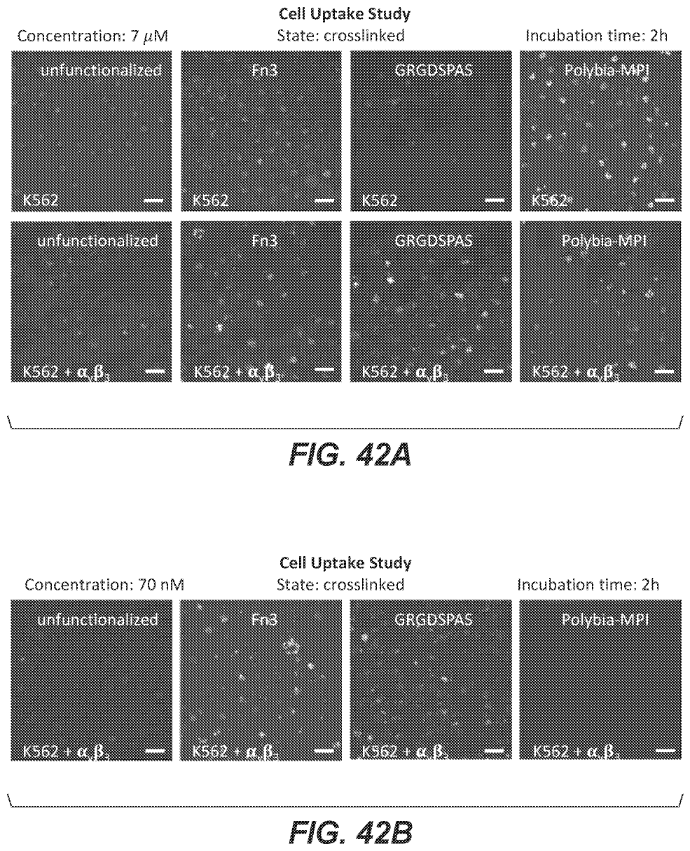

Lastly, cell uptake experiments were performed in two other cell lines. Here, three different ligands were tested: The integrin-targeting ligands Fn3 and GRGDSPAS as well as polybia-MPI. The confocal images taken after 2 hours of co-incubation at 7 μM showed that the crosslinked polybia-MPI particles were actually internalized the most efficiently of all three functionalized constructs (FIG. 42A). Furthermore, the two integrin-targeting ligands both showed increased uptake levels for the αvβ3-displaying K562 variants whereas no significant increase was observed for the native K562 cells in comparison to the unfunctionalized control. In the face of these positive results in the above-CAC regime, all three ligands were then also tested at a concentration of 70 nM—this time only on the αvβ3-positive cell line. Though this concentration might still lie above the construct's CAC, the limit of detection of the cell uptake assay (approx. 10 nM) did not allow us to dilute any further. The resulting confocal images then showed that both GRGDSPAS- and Fn3-carrying nanoparticles were still taken up in significant quantities whereas no increased cell uptake was observed for polybia-MPI (FIG. 42B).

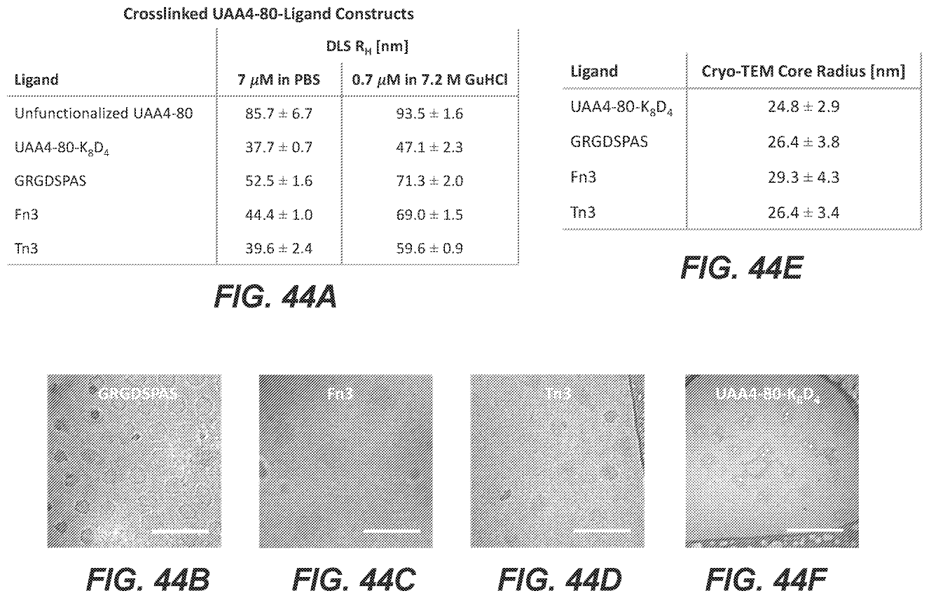

Before testing the effect of nanoscale shape with the worm-like UAA4-80-K8D4-ligand constructs, we characterized their self-assembly: Whereas the addition of these three ligands had not had any significant effect on the DLS readings for the spherical nanoparticles (Table 10), a systematic decrease in hydrodynamic radius by approximately 20 nm was observed for the wormlike constructs (FIG. 44A). In the face of these results and the fact that the Rh value does not represent particles with an elongated morphology very accurately, we turned to cryo-TEM to get a better idea of what might have caused this systematic change. The resulting images then clearly showed that the addition of the K8D4-linker and the three different ligands to the corona of the ELP/RLP diblocks had caused the proteins to take up a spherical rather than a worm-like morphology (FIG. 44B-D). The radius of the particles in the cryo-TEM images was determined at between 20 and 40 nm (FIG. 44F) which is in accordance to the DLS readings when also including the solvated and therefore invisible ELP corona. Though spherical micelles like this had already been observed in some of the cryo-TEM images for the undecorated UAA4-80 construct, they had only been a minor side product (FIG. 44E). The functionalization of these constructs now seems to have strongly shifted this equilibrium towards the low aspect ratio fraction.

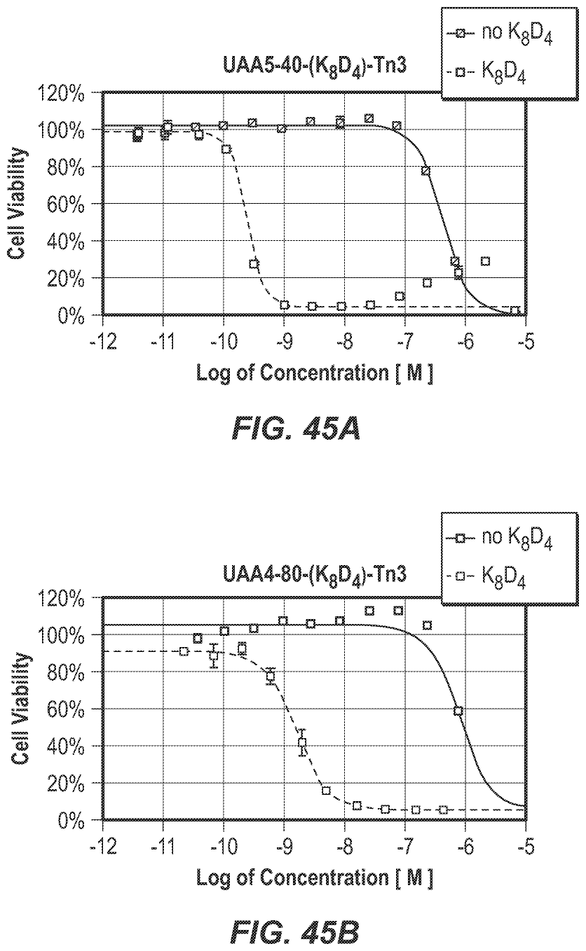

As this change in particle morphology was equally pronounced for the short GRGDSPAS ligand as for the larger Fn3 and Tn3 protein scaffolds, it seems likely that the main cause of this effect was the introduction of the K8D4-linker. Due to the charge and hydrophilicity of this linker it seems reasonable that the attachment of such a peptide to the corona would generally promote lower aspect ratios. The DLS characterization of linker-less UAA4-80 constructs however showed unchanged hydrodynamic radii (FIG. 45A). The subsequently recorded TEM images then confirmed that also the constructs without the K8D4 linker still formed spherical rather than worm-like particles (FIG. 45B-E). This however does not mean that the addition of the linker had no effect on the particle morphology: In fact, the UAA4-80-K8D4 nanoparticles showed an even greater decrease in size than the ligand-carrying constructs (FIG. 45F). This would suggest that self-assembly in the presence of coronal protein, peptide sequence is difficult to determine.

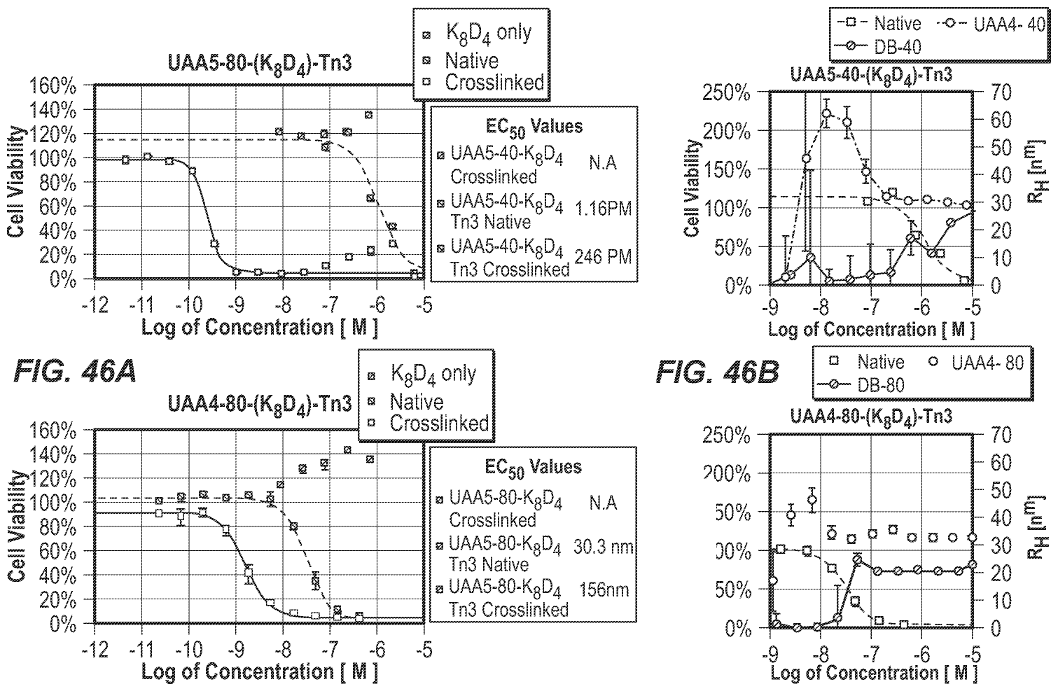

Though the removal of the K8D4 linker did not end up promoting worm-like morphologies, the resulting particles were also no less worm-like than the ones carrying the linker. Thus, the first experiment to evaluate the multivalency benefits of crosslinked nanoparticles was to determine whether there were any differences in binding affinity between the linker-less and linker-carrying constructs. For this, cell viability experiments were performed with crosslinked versions of the Tn3-functionalized constructs. The resulting data showed that the K8D4-containing constructs were significantly more potent than their linker-less analogues for both UAA5-40 and UAA4-80 constructs (FIG. 46). As the Tn3 ligand is a 104-aa protein scaffold and thus would generally not be expected to be at risk of hydrophobic burial, it seems very unexpected that the removal of the linker had such a dramatic influence on the particles' potency. One alternative hypothesis is that this highly charged linker generally helps with cell targeting through electrostatic interaction with the cell membrane. As the cell membrane—particularly in tumor tissue—is negatively charged, it seems plausible that the K8D4 linker with a net charge of +4 could facilitate cell uptake.

What the cell survival plots in FIG. 46 also showed is that the particles with a UAA5-40 basis were generally slightly more potent than the ones constructed from the UAA4-80 diblock. This observation was also somewhat counterintuitive as one would generally expect larger spheres with lower curvatures to have higher contact areas with the cell membrane and therefore bind to the displayed receptors more efficiently. The TEM images in FIG. 44 however showed that the particles with the UAA4-80 basis were not perfect spheres. This in turn indicated that the self-assembly and/or crosslinking of these constructs might be more chaotic than for the UAA5-40 particles and therefore might have also compromised ligand exposure.

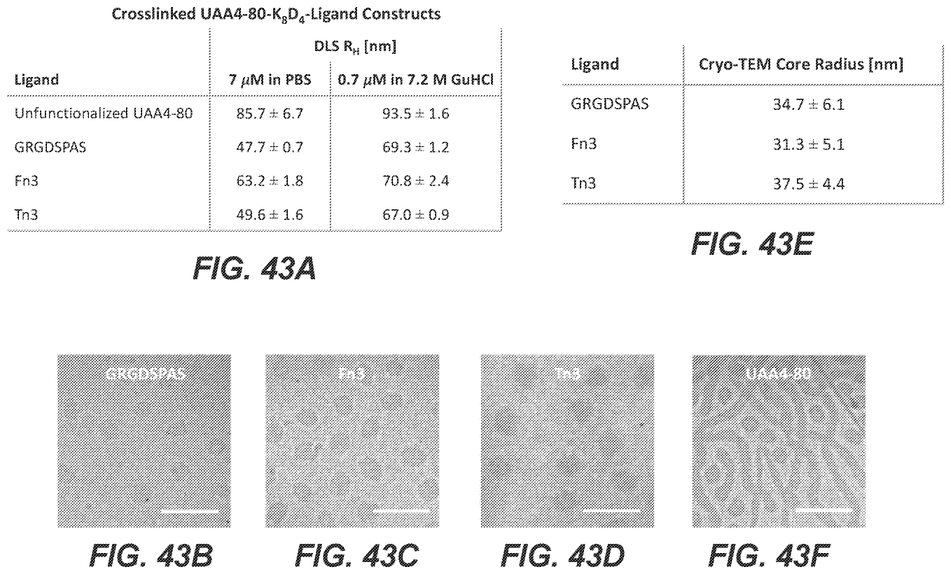

Lastly, the cell survival curve for the crosslinked UAA5-40-K8D4-Tn3 particles in FIG. 46 almost perfectly matched the one from the previous ligand screening experiments (FIG. 43). Whereas it was satisfying to see that the data was reproducible, this also meant that the observed slight increase in cell survival for concentrations around 1 μM was in fact real. Though the cell survival still remained below 30% for all concentrations in this range, this was a rather inexplicable observation. What was particularly confusing is that the cell survival went back to 0% if the concentration was further increased up to 7 μM. As a result, this effect cannot be explained by a high concentration phenomenon such as nanoparticle clustering but was caused by something that exclusively appears at concentrations around 1 μM.

Moving on, we then performed analogous cell viability assays for the corresponding native constructs to quantify the multivalency benefits upon crosslinking. These experiments delivered some encouraging results: For both diblock architectures, the crosslinking significantly increased the potency of the respective nanoformulations (FIG. 46A). For the functionalized UAA5-40 construct, the crosslinking decreased the EC50 value by more than three orders of magnitude. Moreover, a comparison of these cell viability results with the previously recorded CACs for the native UAA5/4-40/80 and DB-40/80 constructs then showed a profound correlation: The EC50 values of the native DR5-targeting constructs almost perfectly matched the CACs of the DB-40 and DB-80 constructs (FIG. 46B). Though it was still largely unclear why the determined CACs for the UAA5/4-40/80 diblocks differed so greatly from their pAzF-free analogues, these observations strongly indicated that those differences were not actually real. It seemed highly unlikely that the near perfect correlation of the EC₅₀ values and the CACs of the DB-40/80 constructs was just a coincidence and cannot be attributed to CAC-dependent particle disassembly. Therefore, this result then represents strong evidence that actually CAC is in the limiting factors to the potency of multivalency-benefitting ligands on self-assembled nanoparticles and that chemical crosslinking is a powerful means to overcome this issue.

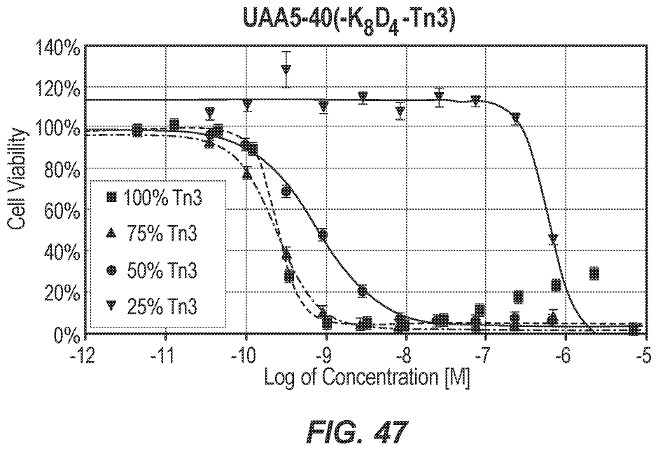

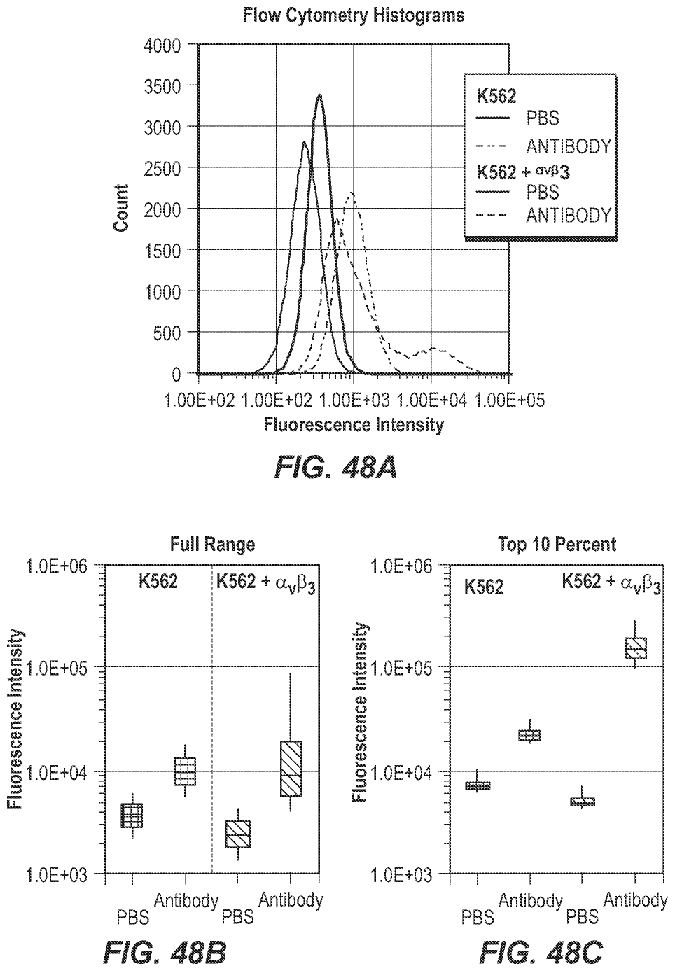

In an additional follow-up experiment we then also investigated how different ligand densities on the crosslinked nanoparticles affected the overall potency. For this we prepared crosslinked particles consisting of both the unfunctionalized UAA5-40 diblock as well as the UAA5-40-K8D4-Tn3 construct at molar ratios of 1:3, 1:1 and 3:1. Subsequently performed cell viability assays with these constructs then showed that a decrease in nanoparticle functionalization down to 50% only led to minor changes in potency (FIG. 47). Only upon further reduction down to 25% functionalized ELP/RLP diblocks within the nanoparticles did the measured EC50 value significantly increase to the high nanomolar range. Due to the non-linearity of the observed trend, we can furthermore assume that the two constructs mixed readily and that the changes in potency are indeed a consequence of decreased ligand density on the nanoparticle surface. If we also consider that the UAA5-40-K8D4-Tn3 construct was not completely pure in the first place as it contained significant amounts of truncation product (FIG. 39A) these observations become even more promising. It seems that at least half of the Tn3 ligands on the fully functionalized nanoparticles could theoretically be removed without compromising the potency of the formulation. This then represents a good starting point for additional engineering of this system for instance towards bispecific nanoparticles.

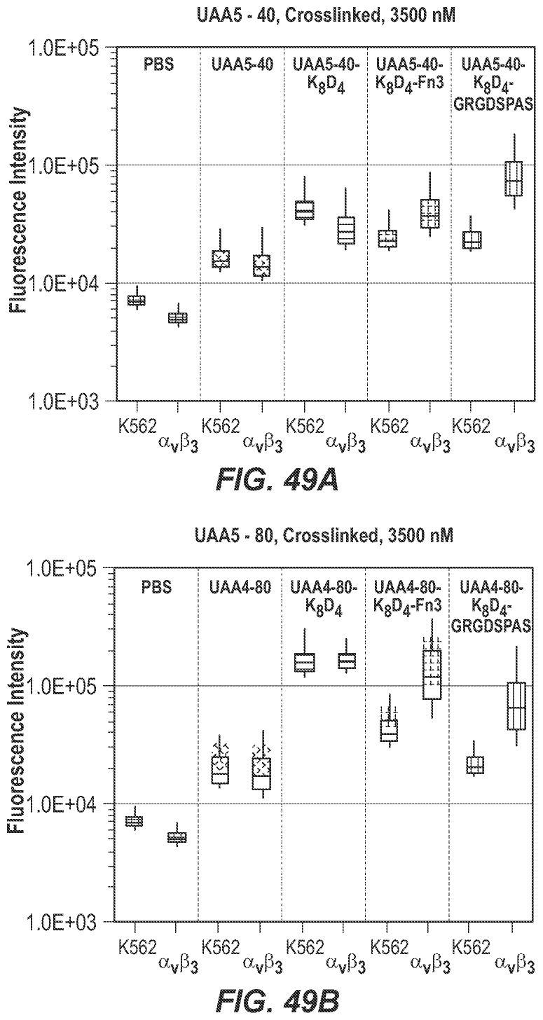

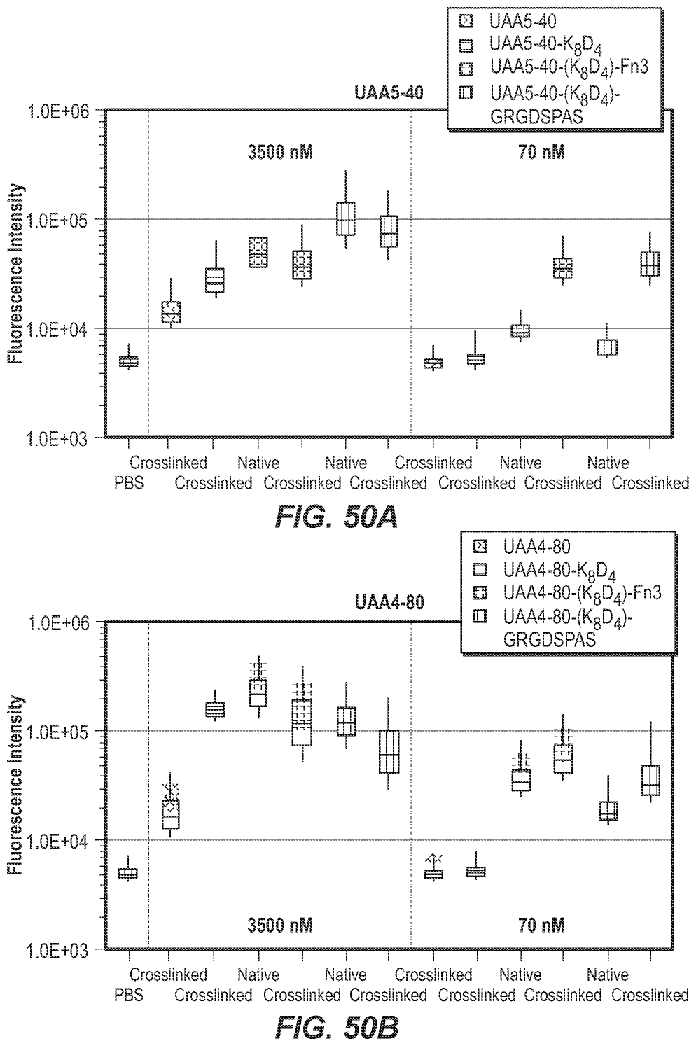

We were interested to evaluate a larger population of cells by flow cytometry. We performed a variety of experiments our variety of protein-based ligands of various sizes at a variety of concentrations in a variety of crosslinking states (FIGS. 48-50). There are a few conclusions from this work. First is that in all cases, we see an increased effect of multivalent display with proteins and peptides alike which is unexpected and non-obvious as they have different mechanisms of binding to the cell surface, are different nominal sizes and can be internalized by a variety of pathways. It is clear that this multivalent effect of cell induced uptake is statistically motivated as even non-specifically targeted cross-linked particles can exhibit high levels of uptake provide there is localized charge on the coronal surface (FIG. 49). However, micelle stability dramatically impacts this cell uptake as reducing concentration of non-crosslinked particles essentially eliminates this non-specific uptake (FIG. 50). We also observe similar effects of particle shape on uptake, although the efficacy of this result appears to be somewhat ligand and concentration dependent, consistent with previous examples (FIG. 48).

Though we were interested in the downstream effects of the treatment with crosslinked nanoparticles, we also wanted to investigate how chemical crosslinking affected the binding to the cell membrane receptors themselves. For this, the binding affinities of crosslinked and native diblock architectures were evaluated using surface plasmon resonance (SPR).

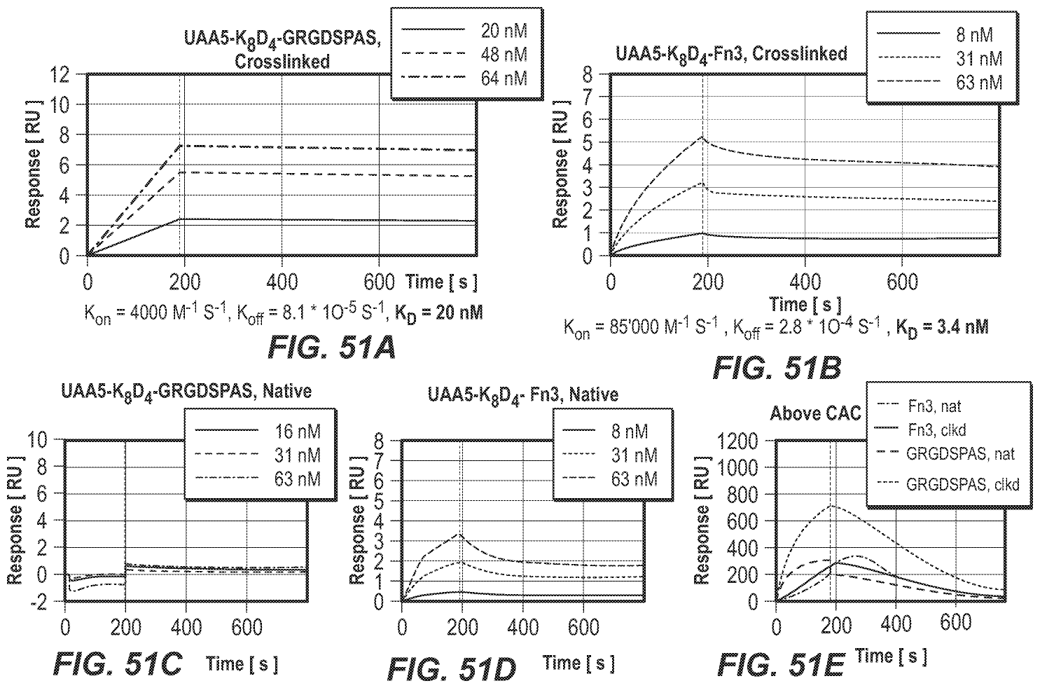

To characterize the integrin-targeting constructs, we started by determining the K_{D} values of the UAA5-40-based constructs. This resulted in unexpectedly tight binding affinities of 3.3 nM and 20 nM for the Fn3 and GRGDSPAS ligands respectively (FIG. 51A). For the peptide ligand specifically, the recorded K_{D} value for the crosslinked nanoparticles also lay significantly below the value for an analogous pAzF-free construct. A closer look at the SPR sensograms describes the mechanism for this effect where the main reason for the increased K_{D} for the ligands is a result of vastly different association rates (kₒₙ) rather than differences in k_{off}.

For their non-crosslinked analogues, no binding was observed for the GRGDSPAS construct whereas some binding-though to a lesser extent than for the crosslinked samples—was measured for the Fn3 variant (FIG. 51B). Based on the results from the cell uptake studies we had expected that the native Fn3 construct might show some binding due to its increased unimer binding affinity compared to the GRGDSPAS variant. At concentrations above the CAC, crosslinking had no significant effect on the binding affinities (FIG. 51C).

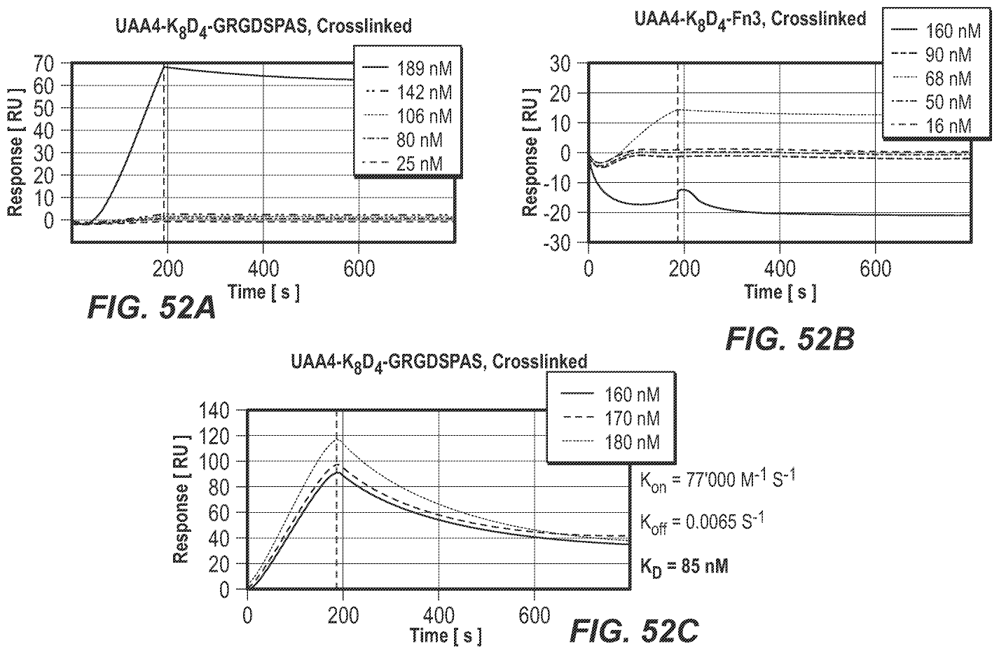

For the integrin-targeting constructs with the larger UAA4-80 construct, the SPR results were unexpected: Whereas the SPR data for the crosslinked GRGDSPAS-functionalized particles showed very strong binding at a concentration of 190 nM, the signal rapidly collapsed upon further dilution (FIG. 52A). For the crosslinked Fn3 constructs on the other hand the sensograms seemed somewhat decoupled from the concentration (FIG. 52B). At 68 nM, the SPR data indicated strong binding whereas at concentrations both above and below that value, no binding was detected. As a result, we could only determine the K_{D} for the GRGDSPAS particles in the mid-nanomolar range which was calculated at 85 nM (FIG. 52C). As neither of the two crosslinked particles showed any detectable binding upon dilution below the CAC, the multivalency benefits upon crosslinking could not be proven for either of these integrin-targeting constructs. This is most probably due to a combination of a comparably low binding affinity for the nanoparticles of this diblock architecture and the low CAC of these ELP/RLP constructs.

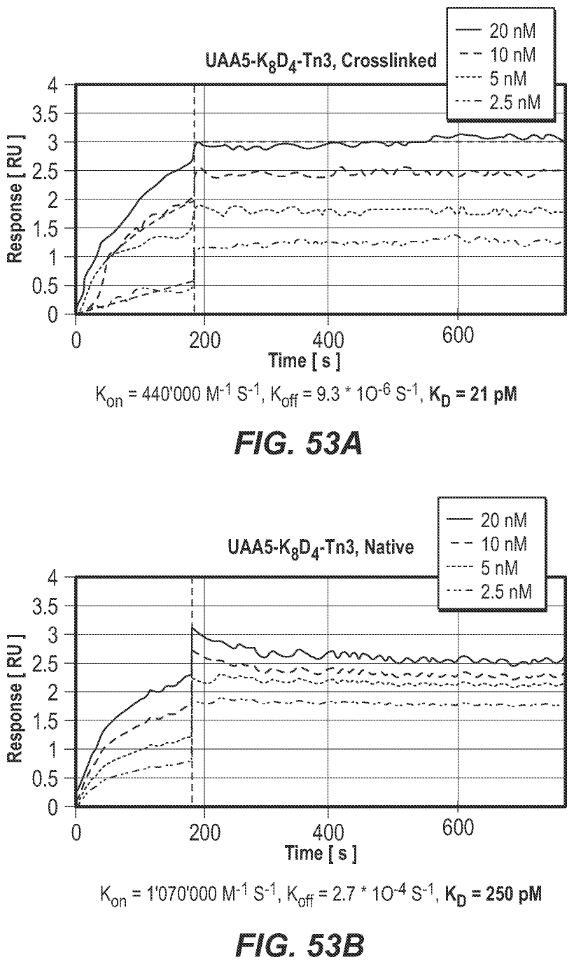

Initial characterization of the crosslinked Tn3 sample with the UAA5-40 basis in the sub-CAC regime yielded a K_{D} value deep in the picomolar range (FIG. 53A). This extremely good binding constant can be mainly attributed to the koff rate which is so low that it is most probably even below the limit of detection of the SPR instrument. As opposed to the integrin-targeting UAA5-40 particles, we however also observed very strong binding for the native Tn3 construct in the same concentration range (FIG. 53B). With 250 nM, the calculated K_{D} value was only 12-fold increased compared to the crosslinked sample. This suggested that crosslinking still improved DR5-binding, it did this only to a very limited degree. Principally, this was not a too unexpected result since we already knew that apoptosis induction via DR5-binding required downstream trimerization of the ligand-bound DR5 receptors in the cell membrane. Therefore, we had expected that the reported multivalency requirement for Tn3-action was at least partly stemming from this mechanistic effect rather than the DR5-binding itself.

### Example 6

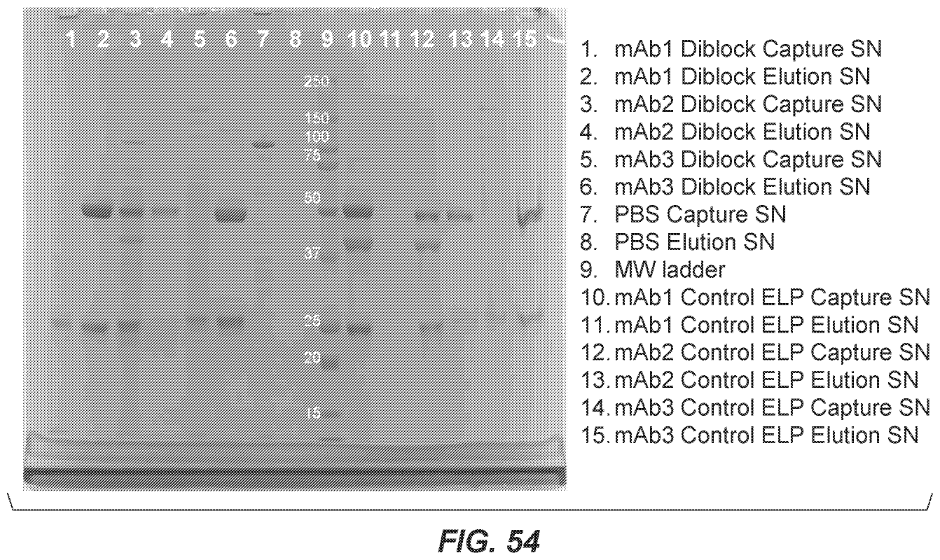

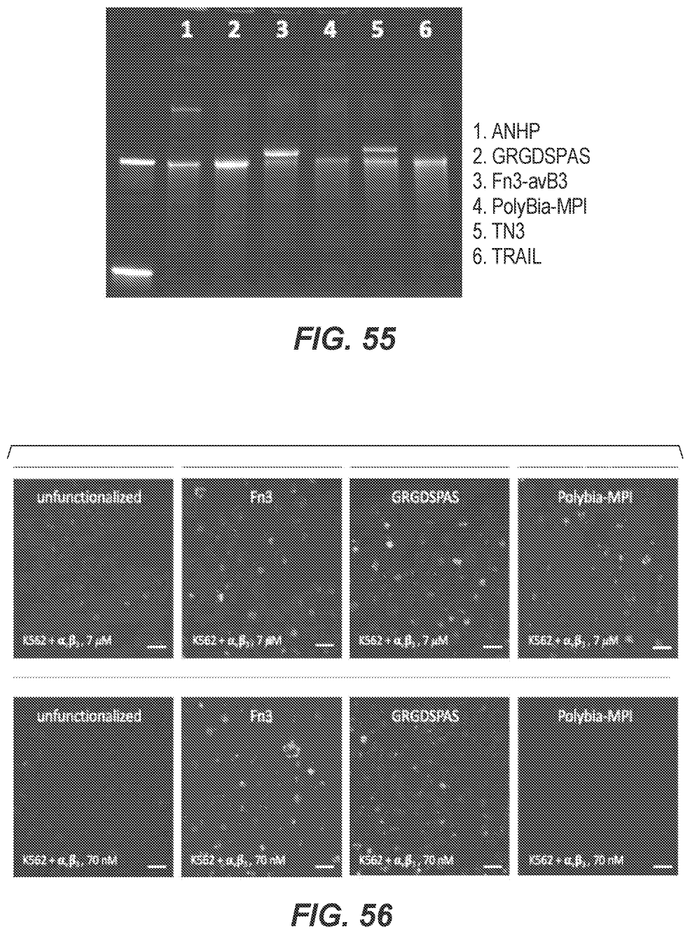

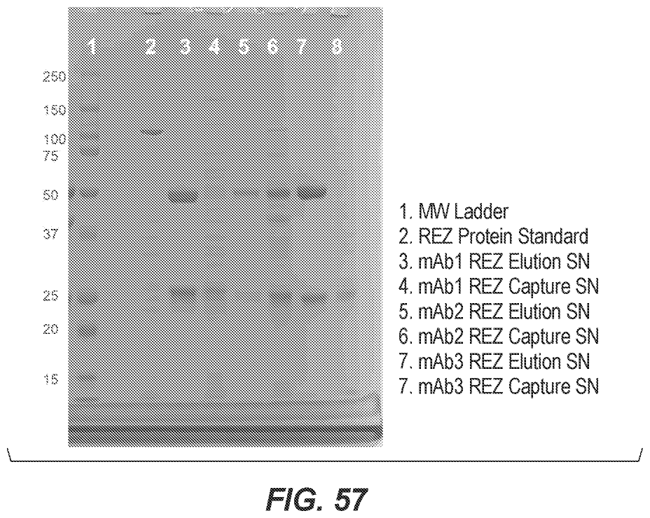

An engineered domain from Staphylococcus aureus (SpA) Protein A (referred to as Z-domain or ZD) was genetically fused to the outside of an RLP-ELP block co-polymer and expressed in E. coli, purified with the method described earlier in the document. To demonstrate the utility of capturing antibodies using the multivalent effect rather than with the traditional, Control ELP, approach, we performed capture and elute experiments in a microfuge format with three different antibodies (mAb). First, we “capture” the mAb which means incubating the RLP-ELP-ZD diblock in the presence of the antibody for ˜1 min at room temperature. We then trigger phase separation by either heating to 37° C. or adding NaCl to the solution. Large aggregates of RLP-ELP-ZD-mAb form which settle over time or can be rapidly pelleted with centrifugation. We then resuspend this sample in a low pH buffer to unbind the mAb from the RLP-ELP-ZD and be released into the elution fraction (Elution SN). Thus, we would expect to see the heavy chain and light chain of the antibody in the Elution SN and the capture SN to be poor with the heavy chain and light chain. As shown in the Control ELP case, the efficacy of this process is variable between three different model antibody compounds, meaning that there is variable levels of capture and elution between the three mAbs (FIG. 54). With the RLP-ELP-ZD construct, the capture SN consistently contains very little mAb suggesting high efficiency capture through increased binding affinity of the ZD to the mAb and the general size of the self-assembled particle reduces variability in the process and can efficiently separate contaminants from the mAb product (other bands seen in lanes 1, 3, and 5).

### Example 7

Dynamic light scattering of cross-linkable protein nanoparticles was performed. At 0.7 μM in 7.2 M GuHCl a non-covalently crosslinked polypeptide dissembles into small unimeric structures that have no self-assembled shape (˜7-10 nM in radius). However, these crosslinked particles remain assembled, actually growing slightly in size due to chain swelling of the core and corona in this new buffer. These novel scaffolds support various targeting domains and assemble into nearly identically sized nanoparticles between 32-52 nM in hydrodynamic radius.

TABLE 12
Crosslinked UAA5-40-K8D4 Ligand Constructs

| Ligand | 7 μM in PBS | 0.7 μM in 7.2M GuHCl |
|---|---|---|
| Unfunctionalized UAA5-40 | 34.3 ± 0.3 | 52.1 ± 1.0 |
| AHNP | 46.7 ± 0.5 | 51.4 ± 0.8 |
| GRGDSPAS | 33.9 ± 0.5 | 48.7 ± 0.7 |
| Fn3 | 37.6 ± 1.2 | 46.4 ± 0.6 |
| Polybia-MPI | 33.8 ± 0.4 | 44.0 ± 1.1 |
| Tn2 | 32.3 ± 0.4 | 47.8 ± 0.7 |

What is claimed:

1. A composition comprising a protein nanoparticle comprising a fusion protein comprising at least one binding polypeptide and at least one unstructured polypeptide.

2. The composition of claim 1, wherein the fusion protein comprises a plurality of unstructured polypeptides.

3. The composition of claim 1, wherein the fusion protein comprises a plurality of targeting polypeptides.

4. The composition of claim 1, wherein the unstructured polypeptides comprise a di-block peptide.

5. The composition of claim 1, wherein the unstructured polypeptides comprise a di-block of a core polypeptide and a corona polypeptide.

6. The composition of claim 1, wherein the unstructured polypeptides comprise COREₙ-CORONAₘ, where n is 20-200 repeats and m is 40-200 repeats.

7. The composition of claim 1, wherein the core polypeptide comprises the sequence QYPSDGRG (SEQ ID NO: 1); GRGDQPYQ (SEQ ID NO: 2); GRGDSPYQ (SEQ ID NO: 3); GRGDSPYS (SEQ ID NO: 4); GRGDQPYS (SEQ ID NO: 5); GRGDSP[3Y:V]S (SEQ ID NO: 6); GRGDSP(Y:V]S (SEQ ID NO: 7); or combinations thereof.

8. The composition of claim 1, wherein the corona polypeptide comprises the sequence VPG[A:G]G (SEQ ID NO: 8); VPGSG (SEQ ID NO: 9); VPGVG (SEQ ID NO: 10); VPQQG (SEQ ID NO: 11); GRGDSPAS (SEQ ID NO: 12); GRGDSPIS (SEQ ID NO: 13); GRGDSPVS (SEQ ID NO: 14); GRGDQPHN (SEQ ID NO: 15); GRGDNPHQ (SEQ ID NO: 16); GRGDSPV (SEQ ID NO: 17); or combinations thereof.

9. The composition of claim 1, wherein the core polypeptide comprises the sequence (RLP)ₙ (SEQ ID NO: 1), where n is 20-200 repeats.

10. The composition of claim 1, wherein the corona polypeptide comprises the sequence (ELP)m (SEQ ID NO: 8), where m is 40-200 repeats.

11. The composition of claim 1, wherein the di-block comprises:
RLP40-ELP40 (SEQ ID NO: 83);
RLP40-ELP80 (SEQ ID NO: 84);
RLP40-ELP160 (SEQ ID NO: 82);
RLP60-ELP80 (SEQ ID NO: 85);
RLP80-ELP80 (SEQ ID NO: 87);
RLP80-ELP160 (SEQ ID NO: 86); or
RLP100-ELP80 (SEQ ID NO: 88).

12. The composition of claim 1, wherein the targeting polypeptide comprises 2 kDa to 100 kDa polypeptide.

13. The composition of claim 1, wherein the targeting polypeptide comprises a type III domain from human fibronectin (Fn3) (SEQ ID NO: 60): aFn3 domain from human tenascin C (Tn3) (SEQ ID NO: 62); or a Z-domain of staphylococcal protein A (SEQ ID NO: 64).

14. The composition of claim 1, wherein the targeting polypeptide comprises a type III domain from human fibronectin (Fn3) (SEQ ID NO: 60).

15. The composition of claim 1, wherein the targeting polypeptide comprises a Fn3 domain from human tenascin C (Tn3) (SEQ ID NO: 62).

16. The composition of claim 1, wherein the targeting polypeptide comprises a Z-domain of staphylococcal protein A with a sequence comprising (SEQ ID NO: 64).

17. The composition of claim 1, wherein the core polypeptide is crosslinked.

18. A protein nanoparticle comprising a fusion protein comprising at least one binding polypeptide and at least one unstructured polypeptide.

19. The protein nanoparticle of claim 18, wherein the fusion protein comprises a plurality of unstructured polypeptides.

20. The protein nanoparticle of claim 18, wherein the fusion protein comprises a plurality of binding polypeptides.

21. The protein nanoparticle of claim 18, wherein the unstructured polypeptides comprise a di-block peptide.

22. The protein nanoparticle of claim 18, wherein the unstructured polypeptides comprise a di-block of a core polypeptide and a corona polypeptide.

23. The protein nanoparticle of claim 18, wherein the unstructured polypeptides comprise COREₙ-CORONAₘ, where n is 20-200 repeats and m is 40-200 repeats.

24. The protein nanoparticle of claim 18, wherein the core polypeptide comprises the sequence QYPSDGRG (SEQ ID NO: 1); GRGDQPYQ (SEQ ID NO: 2); GRGDSPYQ (SEQ ID NO: 3); GRGDSPYS (SEQ ID NO: 4); GRGDQPYS (SEQ ID NO: 5); GRGDSP[3Y:V]S (SEQ ID NO: 6); GRGDSP(Y:V]S (SEQ ID NO: 7); or combinations thereof.

25. The protein nanoparticle of claim 18, wherein the repeating core polypeptide sequence is interspersed with at least 1 but no more than 10 non-canonical amino acids selected from azidophenylalanine, acetylphenylalanine, propargyloxyphenylalanine, acetylphenylalanine, or azidohomoalanine.

26. The protein nanoparticle of claim 18, wherein the corona polypeptide comprises the sequence VPG[A:G]G (SEQ ID NO: 8); VPGSG (SEQ ID NO: 9); VPGVG (SEQ ID NO: 10); VPQQG (SEQ ID NO: 11); GRGDSPAS (SEQ ID NO: 12); GRGDSPIS (SEQ ID NO: 13); GRGDSPVS (SEQ ID NO: 14); GRGDQPHN (SEQ ID NO: 15); GRGDNPHQ (SEQ ID NO: 16); GRGDSPV (SEQ ID NO: 17); or combinations thereof.

27. The protein nanoparticle of claim 18, wherein the core polypeptide comprises the sequence (RLP)ₙ (SEQ ID NO: 1), where n is 20-200 repeats.

28. The protein nanoparticle of claim 18, wherein the corona polypeptide comprises the sequence (ELP)m (SEQ ID NO: 8), where m is 40-200 repeats.

29. The protein nanoparticle of claim 18, wherein the di-block comprises:
RLP40-ELP40 (SEQ ID NO: 83);
RLP40-ELP80 (SEQ ID NO: 84);
RLP40-ELP160 (SEQ ID NO: 82);
RLP60-ELP80 (SEQ ID NO: 85);
RLP80-ELP80 (SEQ ID NO: 87);
RLP80-ELP160 (SEQ ID NO: 86); or
RLP100-ELP80 (SEQ ID NO: 88).

30. The protein nanoparticle of claim 18, wherein the targeting polypeptide comprises 2 kDa to 100 kDa polypeptide.

31. The protein nanoparticle of claim 18, wherein the binding polypeptide comprises a type III domain from human fibronectin (Fn3) (SEQ ID NO: 60); a Fn3 domain from human tenascin C (Tn3) (SEQ ID NO: 62); or a Z-domain of staphylococcal protein A (SEQ ID NO: 64).

32. The protein nanoparticle of claim 18, wherein the binding polypeptide comprises a comprises a type III domain from human fibronectin (Fn3) (SEQ ID NO: 60).

33. The protein nanoparticle of claim 18, wherein the binding polypeptide comprises a Fn3 domain from human tenascin C (Tn3) (SEQ ID NO: 62).

34. The protein nanoparticle of claim 18, wherein the binding polypeptide comprises a Z-domain of staphylococcal protein A with a sequence comprising (SEQ ID NO: 64).

35. The protein nanoparticle of claim 18, wherein the binding polypeptide comprises an ErbB2 receptor binding protein (ANHP) (SEQ ID NO: 74).

36. The protein nanoparticle of claim 18, wherein the binding polypeptide comprises a cell-binding peptide (GRGDSPAS) (SEQ ID NO: 76).

37. The protein nanoparticle of claim 18, wherein the binding polypeptide comprises an adeno associated virus (AAV) binding protein (PKD2) (SEQ ID NO: 112).

38. The protein nanoparticle of claim 18, wherein the binding polypeptide comprises an adenovirus (AdV) binding protein (CAR) (SEQ ID NO: 114).

39. The protein nanoparticle of claim 18, wherein the binding polypeptide comprises a lentivirus (LV) binding protein (CR2) (SEQ ID NO: 116) or (CR3) (SEQ ID NO: 118).

40. The protein nanoparticle of claim 18, wherein the binding polypeptide comprises an albumin binding protein (ABP) (SEQ ID NO: 120).

41. The protein nanoparticle of any one of claims 22-40 where the core is covalently crosslinked using light or other click-chemistry compatible linkers.

42. The protein nanoparticle of any one of claims 22-41, wherein the core polypeptide is crosslinked.

43. The protein nanoparticle of claim 18, wherein the nanoparticle encapsulates one or more small molecule drugs within its interior.

44. The protein nanoparticle of claim 18, wherein the fusion protein further comprises a therapeutic protein.

45. The protein nanoparticle of claim 18, wherein the composition is a therapeutic agent, targeted-delivery agent, separation agent, or purification agent.

46. A therapeutic agent comprising the protein nanoparticle of claim 18.

47. A method of targeting a therapeutic to a cell comprising administering the protein nanoparticle of claim 18.

48. A method of delivering a therapeutic to a cell comprising administering the protein nanoparticle of claim 18.

49. A means for targeting a therapeutic to a cell comprising administering the protein nanoparticle of claim 18.

50. A means for delivering a therapeutic to a cell comprising administering the protein nanoparticle of claim 18.

51. A method for identifying a biomolecule where a protein nanoparticle of claim 18 is added into a solution containing the biomolecule, wherein it specifically binds the biomolecule.

52. A method of purifying a biomolecule comprising using the protein nanoparticle of claim 18 that binds to the biomolecule to isolate the biomolecule from a medium or complex matrix.

53. The method of claim 52, further comprising a triggered phase separation of the binding polypeptide to isolate the biomolecule from contaminants, wherein the trigger is selected from a modulation of temperature, salinity, light, pH, pressure, concentration of the binding polypeptide, concentration of the biomolecule, application of electromagnetic or acoustic waves, or addition of one or more excipients comprising one or more of cofactors, surfactants, crowding reagents, reducing agents, oxidizing agents, denaturing agents, or enzymes.

54. The method of claim 52, further comprising using centrifugation to separate dense phase separated proteins bound to the biomolecule from contaminant biomolecules.

55. The method of claim 52, further comprising using centrifugation to separate phase separated proteins bound to the biomolecule from contaminant biomolecules.

56. The method of claim 52, further comprising using the size of the phase separated droplets to isolate the biomolecule from contaminant species, wherein the size of the binding polypeptide bound to the biomolecule is at least 20 nm in diameter and no larger than 100 μm in diameter.

57. The method of claim 52, wherein the method comprising using flow filtration, membrane chromatography, analytical ultracentrifugation, high performance liquid chromatography, membrane chromatography, normal flow filtration, acoustic wave separation, centrifugation, counterflow centrifugation, and fast protein liquid chromatography to isolation the biomolecule-binding polypeptide complex from contaminant species on the basis of size.

58. The method of claim 52, wherein the biomolecule comprises of at least one of a lipid, a cell, a protein, a nucleic acid, a carbohydrate or a viral particle, wherein the nucleic acid is a single stranded or double stranded DNA or RNA; wherein the viral particle is an adenovirus particle, an adeno-associated virus particle, a lentivirus particle, a retrovirus particle, a poxvirus particle, a measle virus particle, or herpesvirus particle; wherein the protein is human albumin, monoclonal IgG antibodies, or Fc fusion antibodies.


SEQUENCE LISTING (121 sequences)

SEQ ID NO: 1 - protein, 8 aa, Artificial Sequence
  Synthetic
QYPSDGRG

SEQ ID NO: 2 - protein, 8 aa, Artificial Sequence
  Synthetic
GRGDQPYQ

SEQ ID NO: 3 - protein, 8 aa, Artificial Sequence
  Synthetic
GRGDSPYQ

SEQ ID NO: 4 - protein, 8 aa, Artificial Sequence
  Synthetic
GRGDSPYS

SEQ ID NO: 5 - protein, 8 aa, Artificial Sequence
  Synthetic
GRGDQPYS

SEQ ID NO: 6 - protein, 32 aa, Artificial Sequence
  Synthetic
GRGDSPYSGRGDSPYSGRGDSPYSGRGDSPVS

SEQ ID NO: 7 - protein, 16 aa, Artificial Sequence
  Synthetic
GRGDSPYSGRGDSPVS

SEQ ID NO: 8 - protein, 10 aa, Artificial Sequence
  Synthetic
VPGAGVPGGG

SEQ ID NO: 9 - protein, 5 aa, Artificial Sequence
  Synthetic
VPGSG

SEQ ID NO: 10 - protein, 5 aa, Artificial Sequence
  Synthetic
VPGVG

SEQ ID NO: 11 - protein, 5 aa, Artificial Sequence
  Synthetic
VPQQG

SEQ ID NO: 12 - protein, 8 aa, Artificial Sequence
  Synthetic
GRGDSPAS

SEQ ID NO: 13 - protein, 8 aa, Artificial Sequence
  Synthetic
GRGDSPIS

SEQ ID NO: 14 - protein, 8 aa, Artificial Sequence
  Synthetic
GRGDSPVS

SEQ ID NO: 15 - protein, 8 aa, Artificial Sequence
  Synthetic
GRGDQPHN

SEQ ID NO: 16 - protein, 8 aa, Artificial Sequence
  Synthetic
GRGDNPHQ

SEQ ID NO: 17 - protein, 7 aa, Artificial Sequence
  Synthetic
GRGDSPV

SEQ ID NO: 18 - protein, 160 aa, Artificial Sequence
  Synthetic
QYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRG

SEQ ID NO: 19 - DNA, 480 bp, Artificial Sequence
  Synthetic
cagtacccatctgacggtcgcggtcagtatccgagcgacggccgtggtcagtatccgagtgacggccgtggtcaatacccttcggatggtcgtgggcagtacccatctgacggtcgcggtcagtatccgagcgacggccgtggtcagtatccgagtgacggccgtggtcaatacccttcggatggtcgtgggcagtacccatctgacggtcgcggtcagtatccgagcgacggccgtggtcagtatccgagtgacggccgtggtcaatacccttcggatggtcgtgggcagtacccatctgacggtcgcggtcagtatccgagcgacggccgtggtcagtatccgagtgacggccgtggtcaatacccttcggatggtcgtgggcagtacccatctgacggtcgcggtcagtatccgagcgacggccgtggtcagtatccgagtgacggccgtggtcaatacccttcggatggtcgtggg

SEQ ID NO: 20 - protein, 160 aa, Artificial Sequence
  Synthetic
GRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQ

SEQ ID NO: 21 - DNA, 480 bp, Artificial Sequence
  Synthetic
gggcgcggggaccagccatatcaaggaagaggtgaccaaccttatcagggtagaggcgatcagccttatcaaggtcgcggtgatcaaccgtatcaaggtagaggggaccaaccatatcagggaaggggagatcaaccttaccaaggtcggggagatcagccctaccaaggcagaggggatcaaccgtaccaggggaggggtgatcaaccataccagggtcgaggcgaccaaccctatcagggccgaggtgatcagccgtaccaagggcgtggcgaccagccctatcaaggacgtggggatcagccataccaaggacgcggagatcaaccatatcaagggagaggtgatcaaccctatcaaggtaggggtgaccaaccgtatcagggtcgtggagatcagccttaccagggcaggggcgatcaaccctaccagggacgtggtgatcaaccttatcaaggccgtggtgaccagccttatcag

SEQ ID NO: 22 - protein, 161 aa, Artificial Sequence
  Synthetic
GRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQG

SEQ ID NO: 23 - DNA, 483 bp, Artificial Sequence
  Synthetic
gggcgcggtgactctccataccagggtcgtggcgacagcccgtatcagggtcgtggcgacagtccgtatcagggtcgtggtgattcgccttaccaagggcgcggtgactctccataccagggtcgtggcgacagcccgtatcagggtcgtggcgacagtccgtatcagggtcgtggtgattcgccttaccaagggcgcggtgactctccataccagggtcgtggcgacagcccgtatcagggtcgtggcgacagtccgtatcagggtcgtggtgattcgccttaccaagggcgcggtgactctccataccagggtcgtggcgacagcccgtatcagggtcgtggcgacagtccgtatcagggtcgtggtgattcgccttaccaagggcgcggtgactctccataccagggtcgtggcgacagcccgtatcagggtcgtggcgacagtccgtatcagggtcgtggtgattcgccttaccaaggc

SEQ ID NO: 24 - protein, 160 aa, Artificial Sequence
  Synthetic
GRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYS

SEQ ID NO: 25 - DNA, 480 bp, Artificial Sequence
  Synthetic
ggacgcggcgatagtccatattcaggtcggggagatagtccctactcaggtaggggagacagcccatatagcgggcgtggcgactctccatatagtgggagaggggactctccgtactctgggcgcggtgattcaccatattctggtcgaggtgactcaccttattctggcaggggtgatagtccgtattcaggccgtggtgattccccctatagtggaaggggagattcaccgtatagtggtagaggcgacagtccttattcgggacgaggtgattctccttatagtggccgaggggattcaccctattctggaagaggtgatagcccttattcagggaggggcgattctccctattcaggacggggtgactctccttactccggtcgcggagattctccatactcgggtcgtggagactccccatattccggcagaggtgacagtccatacagtggacgtggggatagtccttacagt

SEQ ID NO: 26 - protein, 160 aa, Artificial Sequence
  Synthetic
GRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYS

SEQ ID NO: 27 - DNA, 480 bp, Artificial Sequence
  Synthetic
ggacgaggtgatcagccgtattcgggtcgaggggatcagccatattcaggtcgcggggatcaaccatactcaggtagaggggaccaaccgtattccggtcgtggtgaccaaccatatagcggcaggggtgatcaaccgtatagtggtcggggtgaccaaccctattctggtaggggagatcagccctattcaggacgcggagatcaaccttactctgggagaggtgaccagccttacagtggaaggggcgaccagccatatagtggccgaggcgatcaaccgtactcgggacggggagatcaaccctatagtgggcgcggtgatcaaccctactccgggcgtggcgatcagccatacagcggacgtggtgatcaaccatattctggcagaggtgatcaaccttatagtggacgtggagatcagccttattctggaagaggcgaccaaccttattcaggccgtggggaccagccctacagt

SEQ ID NO: 28 - protein, 160 aa, Artificial Sequence
  Synthetic
GRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVS

SEQ ID NO: 29 - DNA, 480 bp, Artificial Sequence
  Synthetic
ggacgaggggactcaccttatagtgggcgcggcgactctccatattccggtcggggagattcaccatatagtggccgtggcgatagtcctgtgtctggtaggggagactctccgtattcaggacgcggtgattcaccctacagtggacggggtgattctccttattcgggtcgtggggattctccagtttcaggtagaggtgatagcccttattcaggccgaggtgacagtccttactccggcaggggtgactccccatactcgggacgtggtgatagtccagtatcaggaaggggagatagtccctactctgggagaggggatagtccgtactcaggtcgaggtgattccccctattctggaagaggtgactctcctgtatctggcagaggcgacagtccatacagcgggcgtggagattctccctatagtggtcgcggagacagcccatattcagggaggggcgattcacctgttagt

SEQ ID NO: 30 - protein, 160 aa, Artificial Sequence
  Synthetic
GRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVS

SEQ ID NO: 31 - DNA, 480 bp, Artificial Sequence
  Synthetic
ggacgcggagattctccatactctggtcgaggggatagtccggtctcaggaagaggcgatagtccatattcaggacgaggtgacagtccagtatctggcagaggggactctccgtattccggcaggggagacagccctgtttcaggccgaggtgattctccttactcaggtcggggagatagtcctgtgtctggtaggggtgactccccctattctggaaggggagattcacctgtaagtgggcgtggtgattcaccctactcgggtcgtggagactctccagttagtggtcgcggtgattccccatatagcgggcgcggcgattctcccgtttctgggaggggcgacagtccttattcgggacgtggcgactctcctgtatcagggagaggtgatagtccctacagtggccgtggggattcaccagtgtcaggtagaggtgactcaccttatagtggacggggtgatagcccagtcagt

SEQ ID NO: 32 - protein, 160 aa, Artificial Sequence
  Synthetic
GRGDSPASGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPAS

SEQ ID NO: 33 - DNA, 480 bp, Artificial Sequence
  Synthetic
ggccgcggcgattccccagcctccggaagaggtgattcgccagcatctggccgaggggattcacctgcttcggggaggggtgactctccagccagtggcagaggggatagtccggcatccggtagaggggactctcctgcctcgggccgtggagattcaccagctagtggtaggggcgatagtccagcgtcaggacgtggcgacagtcctgcgagcggtcgtggggacagtccagcttcgggtcggggtgattcccctgcaagcggacgaggtgatagtcctgcatcaggtagaggcgactcccccgcctcaggtcgcggagatagccctgcgtctgggagaggtgactcacccgctagtgggcgtggtgattcaccggcttctggtcgaggtgacagcccggcctctggaaggggagattctcccgcatcgggacgcggtgatagcccagcaagtggacggggagactcgcctgctagt

SEQ ID NO: 34 - protein, 160 aa, Artificial Sequence
  Synthetic
GRGDSPISGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPIS

SEQ ID NO: 35 - DNA, 480 bp, Artificial Sequence
  Synthetic
ggccgtggggattctcccatctcgggtcggggtgactctcctatttcgggacggggagatagtccgatttctgggaggggtgatagtcctatatccggtcgcggagactctccaataagcgggcgtggtgattcaccaatttcaggacgcggtgattctcctatcagtggacgaggggatagtccaattagtggtcgaggtgattccccaatatctggccgaggtgacagccctatatcaggccgtggcgattcacccatttccggcagaggggactctccgatctcaggtagaggcgacagtcctattagtgggcgcggcgactcccccataagtggcaggggcgatagtcccatatcaggaaggggagattcaccgatatcagggagaggtgactcacctatatctggtaggggagacagtccaatatcaggtcgtggagattctccaatctctggaagaggtgatagcccaatcagc

SEQ ID NO: 36 - protein, 160 aa, Artificial Sequence
  Synthetic
GRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVS

SEQ ID NO: 37 - DNA, 480 bp, Artificial Sequence
  Synthetic
ggacgaggtgattcgcctgttagtggccgaggggatagtcccgtatctgggaggggcgatagtccggtctcaggtaggggagatagtcctgtatcagggagaggcgactctccagttagtggtcgaggtgacagtccagtgagcggcaggggtgatagtccagtatcaggacggggagattcccctgtgtcaggccgtggcgattctcccgtgtctggaagaggtgatagccctgtctctggtcgcggggactctcctgtgagtggacgcggtgattcaccggtatcaggtcggggtgactcgccggtttcaggaaggggagactccccagtaagcggacgtggagattctcctgtttccgggcgcggagattcaccagtttctggcagaggtgactcacctgtaagtgggcgtggggattcacccgtttcgggtcgtggtgattctccagtcagtggtagaggggacagcccagtgtcg

SEQ ID NO: 38 - protein, 160 aa, Artificial Sequence
  Synthetic
GRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHN

SEQ ID NO: 39 - DNA, 480 bp, Artificial Sequence
  Synthetic
ggtcgtggtgatcaaccccataatggacgcggagatcagccgcataacgggagaggggatcaacctcataatggcagaggggaccaaccacataacggtagaggtgaccagccccataatggtcggggagatcagccccacaacggacgtggcgatcaaccccacaatggccgtggggaccagccacataatggtagaggcgaccaacctcataacggcaggggtgaccaaccccataacggaaggggagatcaaccacacaacgggcgcggggatcagccacacaatggacgtggtgaccaacctcacaacggccgaggtgatcaaccacataatggaagaggtgatcagcctcacaatgggcgtggagatcaaccgcacaatggtcgaggcgatcagcctcataacggtcgcggtgaccaaccgcataatggtaggggcgaccagcctcataatgggaggggtgatcaacctcacaat

SEQ ID NO: 40 - protein, 160 aa, Artificial Sequence
  Synthetic
GRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQ

SEQ ID NO: 41 - DNA, 480 bp, Artificial Sequence
  Synthetic
ggtcgtggtgataatccgcaccagggtagaggcgataacccccatcaaggaagaggggataatccccaccaagggcgcggggataacccgcaccaaggtcggggagataatcctcaccaaggccgtggggacaaccctcatcaaggtagaggtgacaatccgcatcagggacgcggtgataatccacatcaagggagaggtgataatcctcatcagggtaggggagataaccctcatcaggggaggggtgataatccccatcagggccggggcgacaacccccaccagggcaggggtgacaacccgcatcaaggcagaggggacaatccacatcagggtcgcggagacaatcctcatcaaggacgtggcgataatccacaccaggggcgtggagataacccacatcaaggtcgaggtgataaccctcaccagggaaggggagacaacccacaccaaggacgaggcgacaatccccatcaa

SEQ ID NO: 42 - protein, 28 aa, Artificial Sequence
  Synthetic
GRGDSPVGRGDSPVGRGDSPVGRGDSPV

SEQ ID NO: 43 - DNA, 84 bp, Artificial Sequence
  Synthetic
gggcgcggtgactctccagtgggtcgtggcgacagcccggttggtcgtggcgactcgccggtgggtcgtggtgatagtcctgtc

SEQ ID NO: 44 - protein, 200 aa, Artificial Sequence
  Synthetic
GAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVP

SEQ ID NO: 45 - DNA, 600 bp, Artificial Sequence
  Synthetic
ggcgcaggtgttccgggtggcggtgtgccgggcgcaggtgtcccgggtggcggtgtgccgggcgcaggtgtcccgggtggcggtgttccgggcgcaggtgtcccgggtggcggtgtgccgggcgcaggtgttccgggtggcggggtgccgggcgcaggtgttccgggtggcggtgtgccgggcgcaggtgtcccgggtggcggtgtgccgggcgcaggtgtcccgggtggcggtgttccgggcgcaggtgtcccgggtggcggtgtgccgggcgcaggtgttccgggtggcggggtgccgggcgcaggtgttccgggtggcggtgtgccgggcgcaggtgtcccgggtggcggtgtgccgggcgcaggtgtcccgggtggcggtgttccgggcgcaggtgtcccgggtggcggtgtgccgggcgcaggtgttccgggtggcggggtgccgggcgcaggtgttccgggtggcggtgtgccgggcgcaggtgtcccgggtggcggtgtgccgggcgcaggtgtcccgggtggcggtgttccgggcgcaggtgtcccgggtggcggtgtgccgggcgcaggtgttccgggtggcggggtgccg

SEQ ID NO: 46 - protein, 200 aa, Artificial Sequence
  Synthetic
GSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVP

SEQ ID NO: 47 - DNA, 600 bp, Artificial Sequence
  Synthetic
ggcagcggtgttccgggctctggtgtcccaggttccggcgtaccgggcagcggtgttcctggttctggcgtgccgggcagcggtgttccgggctctggtgtcccaggttccggcgtaccgggcagcggtgttcctggttctggcgtgccgggcagcggtgttccgggctctggtgtcccaggttccggcgtaccgggcagcggtgttcctggttctggcgtgccgggcagcggtgttccgggctctggtgtcccaggttccggcgtaccgggcagcggtgttcctggttctggcgtgccgggcagcggtgttccgggctctggtgtcccaggttccggcgtaccgggcagcggtgttcctggttctggcgtgccgggcagcggtgttccgggctctggtgtcccaggttccggcgtaccgggcagcggtgttcctggttctggcgtgccgggcagcggtgttccgggctctggtgtcccaggttccggcgtaccgggcagcggtgttcctggttctggcgtgccgggcagcggtgttccgggctctggtgtcccaggttccggcgtaccgggcagcggtgttcctggttctggcgtgccg

SEQ ID NO: 48 - protein, 200 aa, Artificial Sequence
  Synthetic
GVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVP

SEQ ID NO: 49 - DNA, 600 bp, Artificial Sequence
  Synthetic
ggcgtgggtgttccgggcgtaggtgtcccaggtgtgggcgtaccgggcgttggtgttcctggtgtcggcgtgccgggcgtgggtgttccgggcgtaggtgtcccaggtgtgggcgtaccgggcgttggtgttcctggtgtcggcgtgccgggcgtgggtgttccgggcgtaggtgtcccaggtgtgggcgtaccgggcgttggtgttcctggtgtcggcgtgccgggcgtgggtgttccgggcgtaggtgtcccaggtgtgggcgtaccgggcgttggtgttcctggtgtcggcgtgccgggcgtgggtgttccgggcgtaggtgtcccaggtgtgggcgtaccgggcgttggtgttcctggtgtcggcgtgccgggcgtgggtgttccgggcgtaggtgtcccaggtgtgggcgtaccgggcgttggtgttcctggtgtcggcgtgccgggcgtgggtgttccgggcgtaggtgtcccaggtgtgggcgtaccgggcgttggtgttcctggtgtcggcgtgccgggcgtgggtgttccgggcgtaggtgtcccaggtgtgggcgtaccgggcgttggtgttcctggtgtcggcgtgccg

SEQ ID NO: 50 - protein, 200 aa, Artificial Sequence
  Synthetic
GVPQQGVPQQGVPQQGVPQQGVPQQGVPQQGVPQQGVPQQGVPQQGVPQQGVPQQGVPQQGVPQQGVPQQGVPQQGVPQQGVPQQGVPQQGVPQQGVPQQGVPQQGVPQQGVPQQGVPQQGVPQQGVPQQGVPQQGVPQQGVPQQGVPQQGVPQQGVPQQGVPQQGVPQQGVPQQGVPQQGVPQQGVPQQGVPQQGVPQQ

SEQ ID NO: 51 - DNA, 600 bp, Artificial Sequence
  Synthetic
ggcgtcccgcagcagggtgtaccgcaacagggcgtgcctcagcaaggtgttccgcagcagggggtgccacaacagggcgtcccgcagcagggtgtaccgcaacagggcgtgcctcagcaaggtgttccgcagcagggggtgccacaacagggcgtcccgcagcagggtgtaccgcaacagggcgtgcctcagcaaggtgttccgcagcagggggtgccacaacagggcgtcccgcagcagggtgtaccgcaacagggcgtgcctcagcaaggtgttccgcagcagggggtgccacaacagggcgtcccgcagcagggtgtaccgcaacagggcgtgcctcagcaaggtgttccgcagcagggggtgccacaacagggcgtcccgcagcagggtgtaccgcaacagggcgtgcctcagcaaggtgttccgcagcagggggtgccacaacagggcgtcccgcagcagggtgtaccgcaacagggcgtgcctcagcaaggtgttccgcagcagggggtgccacaacagggcgtcccgcagcagggtgtaccgcaacagggcgtgcctcagcaaggtgttccgcagcagggggtgccacaacag

SEQ ID NO: 52 - protein, 165 aa, Artificial Sequence
  Synthetic
  MOD_RES (33)..(33): X is p-azido-L-phenylalanine
  MOD_RES (66)..(66): X is p-azido-L-phenylalanine
  MOD_RES (99)..(99): X is p-azido-L-phenylalanine
  MOD_RES (132)..(132): X is p-azido-L-phenylalanine
  MOD_RES (165)..(165): X is p-azido-L-phenylalanine
QYPSDGRGQYPSDGRGQYPSDGRGQYPSDGRGXQYPSDGRGQYPSDGRGQYPSDGRGQYPSDGRGXQYPSDGRGQYPSDGRGQYPSDGRGQYPSDGRGXQYPSDGRGQYPSDGRGQYPSDGRGQYPSDGRGXQYPSDGRGQYPSDGRGQYPSDGRGQYPSDGRGX

SEQ ID NO: 53 - DNA, 495 bp, Artificial Sequence
  Synthetic
cagtacccatctgacggtcgcggtcagtatccgagcgacggccgtggtcagtatccgagtgacggccgtggtcaatacccttcggatggtcgtggctagcagtacccatctgacggtcgcggtcagtatccgagcgacggccgtggtcagtatccgagtgacggccgtggtcaatacccttcggatggtcgtggctagcagtacccatctgacggtcgcggtcagtatccgagcgacggccgtggtcagtatccgagtgacggccgtggtcaatacccttcggatggtcgtggctagcagtacccatctgacggtcgcggtcagtatccgagcgacggccgtggtcagtatccgagtgacggccgtggtcaatacccttcggatggtcgtggctagcagtacccatctgacggtcgcggtcagtatccgagcgacggccgtggtcagtatccgagtgacggccgtggtcaatacccttcggatggtcgtggctag

SEQ ID NO: 54 - protein, 322 aa, Artificial Sequence
  Synthetic
  MOD_RES (161)..(161): X is p-azido-L-phenylalanine
  MOD_RES (322)..(322): X is p-azido-L-phenylalanine
QYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGXQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGX

SEQ ID NO: 55 - DNA, 966 bp, Artificial Sequence
  Synthetic
cagtacccatctgacggtcgcggtcagtatccgagcgacggccgtggtcagtatccgagtgacggccgtggtcaatacccttcggatggtcgtgggcagtacccatctgacggtcgcggtcagtatccgagcgacggccgtggtcagtatccgagtgacggccgtggtcaatacccttcggatggtcgtgggcagtacccatctgacggtcgcggtcagtatccgagcgacggccgtggtcagtatccgagtgacggccgtggtcaatacccttcggatggtcgtgggcagtacccatctgacggtcgcggtcagtatccgagcgacggccgtggtcagtatccgagtgacggccgtggtcaatacccttcggatggtcgtgggcagtacccatctgacggtcgcggtcagtatccgagcgacggccgtggtcagtatccgagtgacggccgtggtcaatacccttcggatggtcgtggctagcagtacccatctgacggtcgcggtcagtatccgagcgacggccgtggtcagtatccgagtgacggccgtggtcaatacccttcggatggtcgtgggcagtacccatctgacggtcgcggtcagtatccgagcgacggccgtggtcagtatccgagtgacggccgtggtcaatacccttcggatggtcgtgggcagtacccatctgacggtcgcggtcagtatccgagcgacggccgtggtcagtatccgagtgacggccgtggtcaatacccttcggatggtcgtgggcagtacccatctgacggtcgcggtcagtatccgagcgacggccgtggtcagtatccgagtgacggccgtggtcaatacccttcggatggtcgtgggcagtacccatctgacggtcgcggtcagtatccgagcgacggccgtggtcagtatccgagtgacggccgtggtcaatacccttcggatggtcgtggctag

SEQ ID NO: 56 - protein, 644 aa, Artificial Sequence
  Synthetic
  MOD_RES (161)..(161): X is p-azido-L-phenylalanine
  MOD_RES (322)..(322): X is p-azido-L-phenylalanine
  MOD_RES (483)..(483): X is p-azido-L-phenylalanine
  MOD_RES (644)..(644): X is p-azido-L-phenylalanine
QYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGXQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGXQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGXQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGX

SEQ ID NO: 57 - DNA, 1932 bp, Artificial Sequence
  Synthetic
cagtacccatctgacggtcgcggtcagtatccgagcgacggccgtggtcagtatccgagtgacggccgtggtcaatacccttcggatggtcgtgggcagtacccatctgacggtcgcggtcagtatccgagcgacggccgtggtcagtatccgagtgacggccgtggtcaatacccttcggatggtcgtgggcagtacccatctgacggtcgcggtcagtatccgagcgacggccgtggtcagtatccgagtgacggccgtggtcaatacccttcggatggtcgtgggcagtacccatctgacggtcgcggtcagtatccgagcgacggccgtggtcagtatccgagtgacggccgtggtcaatacccttcggatggtcgtgggcagtacccatctgacggtcgcggtcagtatccgagcgacggccgtggtcagtatccgagtgacggccgtggtcaatacccttcggatggtcgtggctagcagtacccatctgacggtcgcggtcagtatccgagcgacggccgtggtcagtatccgagtgacggccgtggtcaatacccttcggatggtcgtgggcagtacccatctgacggtcgcggtcagtatccgagcgacggccgtggtcagtatccgagtgacggccgtggtcaatacccttcggatggtcgtgggcagtacccatctgacggtcgcggtcagtatccgagcgacggccgtggtcagtatccgagtgacggccgtggtcaatacccttcggatggtcgtgggcagtacccatctgacggtcgcggtcagtatccgagcgacggccgtggtcagtatccgagtgacggccgtggtcaatacccttcggatggtcgtgggcagtacccatctgacggtcgcggtcagtatccgagcgacggccgtggtcagtatccgagtgacggccgtggtcaatacccttcggatggtcgtggctagcagtacccatctgacggtcgcggtcagtatccgagcgacggccgtggtcagtatccgagtgacggccgtggtcaatacccttcggatggtcgtgggcagtacccatctgacggtcgcggtcagtatccgagcgacggccgtggtcagtatccgagtgacggccgtggtcaatacccttcggatggtcgtgggcagtacccatctgacggtcgcggtcagtatccgagcgacggccgtggtcagtatccgagtgacggccgtggtcaatacccttcggatggtcgtgggcagtacccatctgacggtcgcggtcagtatccgagcgacggccgtggtcagtatccgagtgacggccgtggtcaatacccttcggatggtcgtgggcagtacccatctgacggtcgcggtcagtatccgagcgacggccgtggtcagtatccgagtgacggccgtggtcaatacccttcggatggtcgtggctagcagtacccatctgacggtcgcggtcagtatccgagcgacggccgtggtcagtatccgagtgacggccgtggtcaatacccttcggatggtcgtgggcagtacccatctgacggtcgcggtcagtatccgagcgacggccgtggtcagtatccgagtgacggccgtggtcaatacccttcggatggtcgtgggcagtacccatctgacggtcgcggtcagtatccgagcgacggccgtggtcagtatccgagtgacggccgtggtcaatacccttcggatggtcgtgggcagtacccatctgacggtcgcggtcagtatccgagcgacggccgtggtcagtatccgagtgacggccgtggtcaatacccttcggatggtcgtgggcagtacccatctgacggtcgcggtcagtatccgagcgacggccgtggtcagtatccgagtgacggccgtggtcaatacccttcggatggtcgtggctag

SEQ ID NO: 58 - protein, 162 aa, Artificial Sequence
  Synthetic
  MOD_RES (162)..(162): X is p-azido-L-phenylalanine
GRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGX

SEQ ID NO: 59 - DNA, 486 bp, Artificial Sequence
  Synthetic
gggcgcggtgactctccataccagggtcgtggcgacagcccgtatcagggtcgtggcgacagtccgtatcagggtcgtggtgattcgccttaccaagggcgcggtgactctccataccagggtcgtggcgacagcccgtatcagggtcgtggcgacagtccgtatcagggtcgtggtgattcgccttaccaagggcgcggtgactctccataccagggtcgtggcgacagcccgtatcagggtcgtggcgacagtccgtatcagggtcgtggtgattcgccttaccaagggcgcggtgactctccataccagggtcgtggcgacagcccgtatcagggtcgtggcgacagtccgtatcagggtcgtggtgattcgccttaccaagggcgcggtgactctccataccagggtcgtggcgacagcccgtatcagggtcgtggcgacagtccgtatcagggtcgtggtgattcgccttaccaaggctag

SEQ ID NO: 60 - protein, 95 aa, Artificial Sequence
  Synthetic
VSDVPRDLEVVAATPTSLLISWDAPAVTVRYYRITYGETGGNSPVQEFTVPGSKSTATISGLKPGVDYTITVYAVTGRGDSPASSKPISINYRTG

SEQ ID NO: 61 - DNA, 288 bp, Artificial Sequence
  Synthetic
ggcgtgtccgatgtaccccgtgacctggaggtagtagccgccacgcccacatcgttattaatctcatgggacgctcctgcagttactgtaagatattatcggatcacttacggagaaacgggtggaaatagcccggtacaggagttcacggtgccgggctccaagagtactgcaacaatctccggtctgaaaccgggcgtagactacaccattactgtctacgcagtaaccggacgcggtgactcaccggcgtcatccaaacctataagcattaactatcggaccggc

SEQ ID NO: 62 - protein, 88 aa, Artificial Sequence
  Synthetic
AIEVKDVTDTTALITWAKPWVDPPPLWGIELTYGIKDVPGDRTTIDLQQKHTAYSIGNLKPDTEYEVSLISFDPYGMRSKPAKETFTT

SEQ ID NO: 63 - DNA, 264 bp, Artificial Sequence
  Synthetic
gcgattgaagttaaggatgtgacggacaccaccgcactgattacatgggcaaaaccctgggtagacccgccgccgctgtggggaatcgaactgacctatggtattaaggatgttccaggcgatcgcacgaccatcgatctgcaacagaaacacaccgcgtatagtatcgggaaccttaaaccagatacagaatatgaagtttctctcatctcctttgatccgtatggaatgcgttctaaacctgccaaagaaaccttcacgacg

SEQ ID NO: 64 - protein, 59 aa, Artificial Sequence
  Synthetic
GVDNKFNKEQQNAFYEILHLPNLNEEQRNAFIQSLKDDPSQSANLLAEAKKLNDAQAPK

SEQ ID NO: 65 - DNA, 177 bp, Artificial Sequence
  Synthetic
ggcgtggacaataagttcaataaggagcagcagaatgccttttacgagattctccacctgcccaacctcaacgaagagcagagaaatgcgtttatccagagtctgaaagacgacccaagccagtccgcaaacctgctggctgaggccaagaagcttaacgacgcacaggccccaaag

SEQ ID NO: 66 - protein, 15 aa, Artificial Sequence
  Synthetic
GGGGSGGGGSGGGGS

SEQ ID NO: 67 - DNA, 45 bp, Artificial Sequence
  Synthetic
ggcggcggtggctctggcggcggtggtagcggtggaggcggaagc

SEQ ID NO: 68 - protein, 182 aa, Artificial Sequence
  Synthetic
GVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGSGGGGSLPETGG

SEQ ID NO: 69 - DNA, 546 bp, Artificial Sequence
  Synthetic
ggcgttcgtgaacgtggtccgcagcgtgttgcagcacatattaccggcacccgtggtcggagcaataccctgagcagcccgaatagcaaaaatgaaaaagcactgggtcgcaaaattaacagctgggaaagcagccgtagcggtcatagctttctgagcaatctgcatctgcgtaatggtgaactggtgattcatgaaaaaggcttctactatatctacagccagacctattttcgcttccaagaagagattaaagaaaacaccaaaaacgataaacaaatggtgcagtacatctataaatacaccagctatccggatccgattctgctgatgaaaagcgcacgtaatagctgttggagcaaagatgcagaatatggcctgtatagcatttatcagggtggcatctttgaactgaaagaaaacgatcgtattttcgtgagcgtgaccaatgaacatctgatcgatatggatcatgaagccagcttttttggtgcatttctggtgggtggatccggtggcggtggctctctgccggaaaccggtggc

SEQ ID NO: 70 - protein, 18 aa, Artificial Sequence
  Synthetic
GWDCLDNKIGRRQCVRLG

SEQ ID NO: 71 - DNA, 54 bp, Artificial Sequence
  Synthetic
ggctgggattgtcttgacaataaaatcggtcgtcgccaatgtgtgcgtttgggc

SEQ ID NO: 72 - protein, 17 aa, Artificial Sequence
  Synthetic
GKKKKKKKKGGGDDDDG

SEQ ID NO: 73 - DNA, 51 bp, Artificial Sequence
  Synthetic
ggcaagaagaagaagaagaagaagaagggcggcggcgacgacgacgacggc

SEQ ID NO: 74 - protein, 15 aa, Artificial Sequence
  Synthetic
GFCGDGFYACAMDVG

SEQ ID NO: 75 - DNA, 45 bp, Artificial Sequence
  Synthetic
ggcttttgtggagatggattttacgcttgttatatggatgtcggc

SEQ ID NO: 76 - protein, 8 aa, Artificial Sequence
  Synthetic
GRGDSPAS

SEQ ID NO: 77 - DNA, 24 bp, Artificial Sequence
  Synthetic
ggccgcggtgactcaccggcgtca

SEQ ID NO: 78 - protein, 15 aa, Artificial Sequence
  Synthetic
GIDWKKLLDAAKQIL

SEQ ID NO: 79 - DNA, 45 bp, Artificial Sequence
  Synthetic
ggcattgattggaagaagttgttggacgccgccaagcagattttg

SEQ ID NO: 80 - protein, 360 aa, Artificial Sequence
  Synthetic
QYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVP

SEQ ID NO: 81 - protein, 560 aa, Artificial Sequence
  Synthetic
QYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVP

SEQ ID NO: 82 - protein, 1120 aa, Artificial Sequence
  Synthetic
QYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVP

SEQ ID NO: 83 - protein, 520 aa, Artificial Sequence
  Synthetic
QYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVP

SEQ ID NO: 84 - protein, 1120 aa, Artificial Sequence; identical to SEQ ID NO: 82
  Synthetic

SEQ ID NO: 85 - protein, 880 aa, Artificial Sequence
  Synthetic
QYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVP

SEQ ID NO: 86 - protein, 1440 aa, Artificial Sequence
  Synthetic
QYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVP

SEQ ID NO: 87 - protein, 1032 aa, Artificial Sequence
  Synthetic
QYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGGPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVP

SEQ ID NO: 88 - protein, 1192 aa, Artificial Sequence
  Synthetic
QYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGGPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVP

SEQ ID NO: 89 - protein, 720 aa, Artificial Sequence
  Synthetic
QYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVP

SEQ ID NO: 90 - protein, 880 aa, Artificial Sequence
  Synthetic
QYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVP

SEQ ID NO: 91 - protein, 1040 aa, Artificial Sequence
  Synthetic
QYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVP

SEQ ID NO: 92 - protein, 720 aa, Artificial Sequence
  Synthetic
QYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVP

SEQ ID NO: 93 - protein, 880 aa, Artificial Sequence
  Synthetic
QYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVP

SEQ ID NO: 94 - protein, 1040 aa, Artificial Sequence
  Synthetic
QYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVP

SEQ ID NO: 95 - protein, 655 aa, Artificial Sequence
  Synthetic
QYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPVSDVPRDLEVVAATPTSLLISWDAPAVTVRYYRITYGETGGNSPVQEFTVPGSKSTATISGLKPGVDYTITVYAVTGRGDSPASSKPISINYRTG

SEQ ID NO: 96 - protein, 815 aa, Artificial Sequence
  Synthetic
QYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPVSDVPRDLEVVAATPTSLLISWDAPAVTVRYYRITYGETGGNSPVQEFTVPGSKSTATISGLKPGVDYTITVYAVTGRGDSPASSKPISINYRTG

SEQ ID NO: 97 - protein, 1135 aa, Artificial Sequence
  Synthetic
QYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPVSDVPRDLEVVAATPTSLLISWDAPAVTVRYYRITYGETGGNSPVQEFTVPGSKSTATISGLKPGVDYTITVYAVTGRGDSPASSKPISINYRTG

SEQ ID NO: 98 - protein, 1535 aa, Artificial Sequence
  Synthetic
QYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPVSDVPRDLEVVAATPTSLLISWDAPAVTVRYYRITYGETGGNSPVQEFTVPGSKSTATISGLKPGVDYTITVYAVTGRGDSPASSKPISINYRTG

SEQ ID NO: 99 - protein, 615 aa, Artificial Sequence
  Synthetic
QYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGQYPSDGRGGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPVSDVPRDLEVVAATPTSLLISWDAPAVTVRYYRITYGETGGNSPVQEFTVPGSKSTATISGLKPGVDYTITVYAVTGRGDSPASSKPISINYRTG

SEQ ID NO: 100 - protein, 640 aa, Artificial Sequence
  Synthetic
GRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHN

SEQ ID NO: 101 - protein, 960 aa, Artificial Sequence
  Synthetic
GRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHN

SEQ ID NO: 102 - skipped

SEQ ID NO: 103 - protein, 960 aa, Artificial Sequence
  Synthetic
GRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQ

SEQ ID NO: 104 - protein, 640 aa, Artificial Sequence
  Synthetic
GRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVS

SEQ ID NO: 105 - protein, 480 aa, Artificial Sequence
  Synthetic
GRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVS

SEQ ID NO: 106 - protein, 640 aa, Artificial Sequence
  Synthetic
GRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVS

SEQ ID NO: 107 - protein, 800 aa, Artificial Sequence
  Synthetic
GRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVS

SEQ ID NO: 108 - protein, 640 aa, Artificial Sequence
  Synthetic
GRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPAS

SEQ ID NO: 109 - protein, 640 aa, Artificial Sequence
  Synthetic
GRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPIS

SEQ ID NO: 110 - protein, 880 aa, Artificial Sequence
  Synthetic
GRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVP

SEQ ID NO: 111 - DNA, 294 bp, Artificial Sequence
  Synthetic
aatcgtccgcccatcgctattgtcagccctcaatttcaggagatcagcttgccgacaacctccactgtaattgatggttcccagtcgaccgacgacgataaaatcgtacaataccactgggaggaactcaaggggccgctgcgtgaggaaaagatcagcgaggataccgcaattttgaaacttagtaaactggttcccggcaactacacattttctttaacggttgtagactcggatggtgccaccaactctacgaccgctaatcttacagtgaacaaagcggtcgattatccg

SEQ ID NO: 112 - protein, 98 aa, Artificial Sequence
  Synthetic
NRPPIAIVSPQFQEISLPTTSTVIDGSQSTDDDKIVQYHWEELKGPLREEKISEDTAILKLSKLVPGNYTFSLTVVDSDGATNSTTANLTVNKAVDYP

SEQ ID NO: 113 - DNA, 129 bp, Artificial Sequence
  Synthetic
ggtccgctgggcagcgttacgtgcaaaagcggcgatttcagctgcggtggccgcgttaatcgctgcatcccgcagttctggcgctgcgatggccaagttgattgcgataatggcagcgatgaacaaggc

SEQ ID NO: 114 - protein, 43 aa, Artificial Sequence
  Synthetic
GPLGSVTCKSGDFSCGGRVNRCIPQFWRCDGQVDCDNGSDEQG

SEQ ID NO: 115 - DNA, 111 bp, Artificial Sequence
  Synthetic
acgtgcagccaagatgagttccgctgtcatgatggcaaatgcatcagccgccagttcgtttgcgatagcgatcgcgattgtctggatggcagcgatgaagccagctgcccg

SEQ ID NO: 116 - protein, 37 aa, Artificial Sequence
  Synthetic
TCSQDEFRCHDGKCISRQFVCDSDRDCLDGSDEASCP

SEQ ID NO: 117 - DNA, 366 bp, Artificial Sequence
  Synthetic
ggcatcaccaccccagaagagatgatcgagaaggccaaaggcgaaaccgcctatctgccgtgcaaattcacgctgagcccggaagatcaaggcccactggatatcgaatggctgatcagcccggccgacaatcagaaggtggaccaagttatcattctgtacagcggcgacaagatctacgatgactactacccggatctgaaaggccgcgtgcacttcaccagcaacgatctgaaaagcggtgatgccagcatcaacgtgaccaatctgcagctgagcgatatcggcacctaccagtgcaaggtgaagaaagcgccgggcgttgcgaacaagaagatccatctggttgtgctggtgaaaccgagc

SEQ ID NO: 118 - protein, 122 aa, Artificial Sequence
  Synthetic
GITTPEEMIEKAKGETAYLPCKFTLSPEDQGPLDIEWLISPADNQKVDQVIILYSGDKIYDDYYPDLKGRVHFTSNDLKSGDASINVTNLQLSDIGTYQCKVKKAPGVANKKIHLVVLVKPS

SEQ ID NO: 119 - DNA, 144 bp, Artificial Sequence
  Synthetic
ctggcggaagctaaagtgcttgcgaatcgcgaactggataaatatggtgttagcgacttttacaagcgtttgattaacaaagccaaaaccgtggaaggcgtcgaggcgctgaaattacatatcctggcagccctgccgggcggc

SEQ ID NO: 120 - protein, 48 aa, Artificial Sequence
  Synthetic
LAEAKVLANRELDKYGVSDFYKRLINKAKTVEGVEALKLHILAALPGG

SEQ ID NO: 121 - DNA, 5298 bp, Artificial Sequence
  Synthetic
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggaggagtacatatgggctgatgataatgatcttcaggatccgaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat